(19) United States
(12) Patent Application Publication
(10) Pub. No.: US 2023/0190954 A1
(43) Pub. Date: Jun. 22, 2023

(54) COMPOSITION AND METHODS FOR TREATMENT OF PRIMARY CILIARY DYSKINESIA

(71) Applicant: TRANSLATE BIO, INC., Waltham, MA (US)

(72) Inventors: Caroline J. WOO, Cambridge, MA (US); Anusha DIAS, Cambridge, MA (US); Khang Anh TRAN, Cambridge, MA (US); Lianne BOEGLIN, Cambridge, MA (US); Nicholas K. CLARK, Cambridge, MA (US); John ANDROSAVICH, Cambridge, MA (US); Shraddha SHARMA, Cambridge, MA (US); Gang SUN, Cambridge, MA (US); Neha KAUSHAL, Cambridge, MA (US); Shrirang KARVE, Cambridge, MA (US)

(21) Appl. No.: 17/923,839

(22) PCT Filed: May 7, 2021
(86) PCT No.: PCT/US2021/031305

§ 371 Date: Nov. 7, 2022

Related U.S. Application Data
(60) Provisional application No. 63/021,365, filed on May 7, 2020.
(60) Provisional application No. 63/111,352, filed on Nov. 9, 2020.
(60) Provisional application No. 63/128,770, filed on Dec. 21, 2020.

(51) Int. Cl. A61K 48/00; A61K 38/17; A61K 47/24; A61K 9/00; A61K 47/22; A61K 47/18

(52) U.S. Cl. CPC A61K 48/0008; A61K 9/0078; A61K 38/1709; A61K 47/22; A61K 47/24; A61K 47/183; A61K 48/0066; A61K 48/0075; A61K 48/0083; A61P 11/00

(57) ABSTRACT

The present invention provides, among other things, methods and compositions for treating primary ciliary dyskinesia (PCD) based on mRNA therapy. The compositions used in treatment of PCD comprise an mRNA comprising a dynein axonemal intermediate chain 1 (DNAI1) coding sequence and are administered at an effective dose and an administration interval such that at least one symptom or feature of PCD is reduced in intensity, severity, or frequency or has a delayed onset. mRNAs with optimized DNAI1 coding sequences are provided that can be administered without the need for modifying the nucleotides of the mRNA to achieve sustained in vivo function.

### CROSS-REFERENCE TO RELATED APPLICATIONS

This application is a 35 U.S.C. § 371 National Stage Application of International Application No. PCT/US2021/031305, filed on May 7, 2021, which claims priority to, and the benefit of, U.S. Provisional Application No. 63/021,365, filed on May 7, 2020, U.S. Provisional Application No. 63/111,352, filed on Nov. 9, 2020, and U.S. Provisional Application No. 63/128,770, filed on Dec. 21, 2020. The contents of each of the foregoing applications are hereby incorporated by reference in their entireties.

### INCORPORATION-BY-REFERENCE OF SEQUENCE LISTING

The present specification makes reference to a Sequence Listing (submitted electronically as a .txt file named MRT-2145WO_ST25 on May 7, 2021). The .txt file was generated on Apr. 28, 2021 and is 39 KB in size. The entire contents of the sequence listing are herein incorporated by reference.

### BACKGROUND

Primary ciliary dyskinesia (PCD) is an auto recessive disorder characterized by abnormal cilia and flagella that are found in the linings of the airway, the reproductive system, and other organs and tissues. PCD occurs in approximately 1 in 16,000. Symptoms are present as early as at birth, with breathing problems, and the affected individuals develop frequent respiratory tract infections beginning in early childhood. People with PCD also have year-round nasal congestion and chronic cough. Chronic respiratory tract infections can result in condition called bronchiectasis, which damages the passages, called bronchi, and can cause life-threatening breathing problems. Some individuals with PCD also have infertility, recurrent ear infections, abnormally placed organs within their chest and abdomen.

Among several genes confirmed to be directly involved in PCD pathogenesis, a significant number of mutations are found in two genes: DNAI1 and DNAH5, encoding intermediate and heavy chains of the axonemal dynein, respectively. Mutations in other genes, coding for proteins involved in the axonemal ultrastructure (DNAH11, DNAI2, TXNDC3, RSPH9, RSPH4A) or assembly (KTU, CRRC50), also have been reported, as well as mutations in the RPGR gene in certain cases of PCD. Mutations in DNAI1 and DNAH5, both associated with a ciliary outer dynein arm (ODA) defect phenotype, are collectively estimated to account for almost 40% of PCD cases.

There is currently no cure for PCD. Current standard of care includes aggressive measures to enhance clearance of mucus and with antibiotic therapy for bacterial infections of the airways. Routine immunizations are administered to prevent respiratory infections and other secondary complications. For some patients, lobectomy, lung transplantation, and sinus surgery are considered. Gene therapy has been studied to address the urgent need for new, more effective treatments of PCD. However, conventional gene therapy methods to deliver the therapeutic agent to the lungs, and specifically to cilia, still remain challenging.

### SUMMARY OF THE INVENTION

The present invention provides, among other things, methods and compositions for use in the treatment of primary ciliary dyskinesia (PCD). The present invention is based, in part, on the surprising discovery that DNAI1 mRNA can be successfully delivered to target tissues in vivo, expressed in target tissue cells and localized to and throughout the cilia, resulting in DNAI1 protein expression detectable throughout the length of cilia. Notably, codon-optimized mRNAs encoding DNAI1 proteins described herein provide an increased DNAI1 protein expression and activity level. Furthermore, cationic lipids described herein exhibit increased potency for pulmonary delivery and increased DNAI1 expression.

In one aspect, the present invention provides, among other things, a method of delivery of a dynein axonemal intermediate chain 1 (DNAI1) protein, comprising administering to a subject in need of delivery an mRNA encoding the DNAI protein.

In one aspect, the present invention provides a method of treating primary ciliary dyskinesia (PCD) comprising administering to a subject in need of treatment an mRNA encoding human axonemal dynein intermediate chain 1 (DNAI1) at an effective dose and an administration interval such that at least one symptom or feature of PCD is reduced in intensity, severity, or frequency or has delayed in onset.

In some embodiments, the DNAIlmRNA is encapsulated in a liposome.

In some embodiments, the liposome comprises one or more cationic lipids, one or more non-cationic lipids and one or more PEG-modified lipids. In some embodiments, the liposome comprises no more than three distinct lipid components. In some embodiments, the liposome comprises no more than four distinct lipid components.

In some embodiments, the one or more cationic lipids are selected from the group consisting of TL1-01D-DMA, TL1-10D-DMA, GL-TES-SA-DMP-E18-2, HEP-E4-E10, HEP-E3-E10, TL1-04D-DMA, GL-TES-SA-DME-E18-2, Guan-SS-Chol, SY-3-E14-DMAPr, RL3-07D-DMA, cKK-E12, OF-02, ICE (Imidazol-based ester) and combinations thereof.

In some embodiments, the cationic lipid is TL1-01D-DMA. In some embodiments, the cationic lipid is TL1-10D-DMA. In some embodiments, the cationic lipid is GL-TES-SA-DMP-E18-2. In some embodiments, the cationic lipid is HEP-E4-E10. In some embodiments, the cationic lipid is HEP-E3-E10. In some embodiments, the cationic lipid is TL1-04D-DMA. In some embodiments, the cationic lipid is GL-TES-SA-DME-E18-2. In some embodiments, the cationic lipid is Guan-SS-Chol. In some embodiments, the cationic lipid is SY-3E14-DMAPr. In some embodiments, the cationic lipid is RL3-07D-DMA. In some embodiments, the cationic lipid is ICE.

In some embodiments, the one or more non-cationic lipids are selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DOPC (1,2-dioleyl- sn-glycero-3 -pho sphotidylcholine) DPPE (1,2-dipalmitoyl- sn-glycero-3-phosphoethanolamine), DMPE (1,2-dimyristoyl-sn-glycero-3-phosphoethanolamine), DOPG (1,2-dioleoyl-sn-glycero-3-phospho-(1′-rac-glycerol)) or combinations thereof. In some embodiments, the non-cationic lipid is DOPE.

In some embodiments, the one or more PEG-modified lipids comprise a poly(ethylene) glycol chain of up to 5 kDa in length covalently attached to a lipid with alkyl chain(s) of C6-C20 length. In some embodiments, the PEG-modified lipid is DMG-PEG2K.

In some embodiments, the cationic lipid constitutes about 30-60 % of the liposome by molar ratio.

In some embodiments, the cationic lipid constitutes about 30%, 40 %, 50%, or 60% of the liposome by molar ratio.

In some embodiments, the liposomes comprises four distinct lipid components, namely a cationic lipid, a non-cationic lipid, cholesterol and a PEG-modified lipid. In some embodiments, the molar ratio of cationic lipid to non-cationic lipid to cholesterol to PEG-modified lipid is between about 30-60:25-35:20-30:1-15, respectively.

In some embodiments, the liposomes comprises three distinct lipid components, namely a cationic lipid (typically a sterol-based cationic lipid), a non-cationic lipid, and a PEG-modified lipid. In some embodiments, the molar ratio of cationic lipid to non-cationic lipid to PEG-modified lipid is approximately 60:35:5, respectively.

In some embodiments, the liposome has a size of about 30 nm to 200 nm, optionally wherein the liposome has a size of about 100 nm or less than 100 nm.

In some embodiments, the DNAI1 mRNA is codon optimized. In some embodiments, the codon-optimized mRNA produces at least 10% more, 15% more, 20% more, 25% more, or at least 30% more DNAI1 protein in comparison to a non-codon-optimized mRNA sequence. In some embodiments, the codon-optimized mRNA produces at least 10% more DNAI1 protein in comparison to a non-codon-optimized mRNA sequence. In some embodiments, the codon-optimized mRNA produces at least 15% more DNAI1 protein in comparison to a non-codon-optimized mRNA sequence. In some embodiments, the codon-optimized mRNA produces at least 20% more DNAI1 protein in comparison to a non-codon-optimized mRNA sequence. In some embodiments, the codon-optimized mRNA produces at least 25% more DNAI1 protein in comparison to a non-codon-optimized mRNA sequence. In some embodiments, the codon-optimized mRNA produces at least 30% more DNAI1 protein in comparison to a non-codon-optimized mRNA sequence. In some embodiments, the codon-optimized mRNA produces greater than 30% more DNAI1 protein in comparison to a non-codon-optimized mRNA sequence.

In some embodiments, the DNAI1 mRNA is codon optimized to include a high-producing codon optimized mRNA sequence that provides at least 10% more, 15% more, 20% more, 25% more, or at least 30% more DNAI1 protein in comparison to a second DNAI1 mRNA sequence that is codon optimized with a different, reference codon optimized sequence and delivered at the same dose. In some embodiments, the high-producing codon-optimized mRNA sequence provides at least 10% more DNAI1 protein in comparison to a second DNAI1 mRNA sequence that is codon optimized with a different, reference codon optimized sequence and delivered at the same dose. In some embodiments, the high-producing codon-optimized mRNA sequence provides at least 15% more DNAI1 protein in comparison to a second DNAI1 mRNA sequence that is codon optimized with a different, reference codon optimized sequence and delivered at the same dose. In some embodiments, the high-producing codon-optimized mRNA sequence provides at least 20% more DNAI1 protein in comparison to a second DNAI1 mRNA sequence that is codon optimized with a different, reference codon optimized sequence and delivered at the same dose. In some embodiments, the high-producing codon-optimized mRNA sequence provides at least 25% more DNAI1 protein in comparison to a second DNAI1 mRNA sequence that is codon optimized with a different, reference codon optimized sequence and delivered at the same dose. In some embodiments, the high-producing codon-optimized mRNA sequence provides at least 30% more DNAI1 protein in comparison to a second DNAI1 mRNA sequence that is codon optimized with a different, reference codon optimized sequence and delivered at the same dose. In some embodiments, the high-producing codon-optimized mRNA sequence provides greater than 30% more DNAI1 protein in comparison to a second DNAI1 mRNA sequence that is codon optimized with a different, reference codon optimized sequence and delivered at the same dose.

In some embodiments, the DNAI1 mRNA comprises one or more modified nucleotides.

In some embodiments, the one or more modified nucleotides are selected from pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and/or 2-thiocytidine.

In some embodiments, the mRNA is unmodified.

In some embodiments, the mRNA comprises a 5′-untranslated region (5′-UTR) that has a sequence set forth in SEQ ID NO: 2 or SEQ ID NO: 3.

In some embodiments, the mRNA comprises a 3′-untranslated region (3′-UTR) that has a sequence set forth in SEQ ID NO: 4 or SEQ ID NO: 5.

In some embodiments, the mRNA comprises a coding sequence at least 70%, 75%, 80%, 85%, 90%, or 95% identical to any one of SEQ ID NO: 6 to SEQ ID NO: 10. In some embodiments, the mRNA comprises a coding sequence at least 70% identical to any one of SEQ ID NO: 6 to SEQ ID NO: 10. In some embodiments, the mRNA comprises a coding sequence at least 80% identical to any one of SEQ ID NO: 6 to SEQ ID NO: 10. In some embodiments, the mRNA comprises a coding sequence at least 90% identical to any one of SEQ ID NO: 6 to SEQ ID NO: 10. In some embodiments, the mRNA comprises a coding sequence at least 95% identical to any one of SEQ ID NO: 6 to SEQ ID NO: 10. In some embodiments, the mRNA comprises a coding sequence at least 96%, at least 97%, at least 98%, or at least 99% identical to SEQ ID NO: 6 to SEQ ID NO: 10. In some embodiments, the mRNA comprises a coding sequence set forth in SEQ ID NO: 6 to SEQ ID NO: 10.

In some embodiments, the mRNA comprises a coding sequence at least 70%, 75%, 80%, 85%, 90%, or 95% identical to SEQ ID NO: 6. In some embodiments, the mRNA comprises a coding sequence at least 70% identical to SEQ ID NO: 6. In some embodiments, the mRNA comprises a coding sequence at least 80% identical to SEQ ID NO: 6. In some embodiments, the mRNA comprises a coding sequence at least 90% identical to SEQ ID NO: 6. In some embodiments, the mRNA comprises a coding sequence at least 95% identical to SEQ ID NO: 6. In some embodiments, the mRNA comprises a coding sequence at least 96%, at least 97%, at least 98%, or at least 99% identical to SEQ ID NO: 6. In some embodiments, the mRNA comprises a coding sequence set forth in SEQ ID NO: 6.

In some embodiments, the mRNA comprises a coding sequence at least 70%, 75%, 80%, 85%, 90%, or 95% identical to SEQ ID NO: 7. In some embodiments, the mRNA comprises a coding sequence at least 70% identical to SEQ ID NO: 7. In some embodiments, the mRNA comprises a coding sequence at least 80% identical to SEQ ID NO: 7. In some embodiments, the mRNA comprises a coding sequence at least 90% identical to SEQ ID NO: 7. In some embodiments, the mRNA comprises a coding sequence at least 95% identical to SEQ ID NO: 7. In some embodiments, the mRNA comprises a coding sequence at least 96%, at least 97%, at least 98%, or at least 99% identical to SEQ ID NO: 7. In some embodiments, the mRNA comprises a coding sequence set forth in SEQ ID NO: 7.

In some embodiments, the mRNA comprises a coding sequence at least 70%, 75%, 80%, 85%, 90%, or 95% identical to SEQ ID NO: 8. In some embodiments, the mRNA comprises a coding sequence at least 70% identical to SEQ ID NO: 8. In some embodiments, the mRNA comprises a coding sequence at least 80% identical to SEQ ID NO: 8. In some embodiments, the mRNA comprises a coding sequence at least 90% identical to SEQ ID NO: 8. In some embodiments, the mRNA comprises a coding sequence at least 95% identical to SEQ ID NO: 8. In some embodiments, the mRNA comprises a coding sequence at least 96%, at least 97%, at least 98%, or at least 99% identical to SEQ ID NO: 8. In some embodiments, the mRNA comprises a coding sequence set forth in SEQ ID NO: 8.

In some embodiments, the mRNA comprises a coding sequence at least 70%, 75%, 80%, 85%, 90%, or 95% identical to SEQ ID NO: 9. In some embodiments, the mRNA comprises a coding sequence at least 70% identical to SEQ ID NO: 9. In some embodiments, the mRNA comprises a coding sequence at least 80% identical to SEQ ID NO: 9. In some embodiments, the mRNA comprises a coding sequence at least 90% identical to SEQ ID NO: 9. In some embodiments, the mRNA comprises a coding sequence at least 95% identical to SEQ ID NO: 9. In some embodiments, the mRNA comprises a coding sequence at least 96%, at least 97%, at least 98%, or at least 99% identical to SEQ ID NO: 9. In some embodiments, the mRNA comprises a coding sequence set forth in SEQ ID NO: 9.

In some embodiments, the mRNA comprises a coding sequence at least 70%, 75%, 80%, 85%, 90%, or 95% identical to SEQ ID NO: 10. In some embodiments, the mRNA comprises a coding sequence at least 70% identical to SEQ ID NO: 10. In some embodiments, the mRNA comprises a coding sequence at least 80% identical to SEQ ID NO: 10. In some embodiments, the mRNA comprises a coding sequence at least 90% identical to SEQ ID NO: 10. In some embodiments, the mRNA comprises a coding sequence at least 95% identical to SEQ ID NO: 10. In some embodiments, the mRNA comprises a coding sequence at least 96%, at least 97%, at least 98%, or at least 99% identical to SEQ ID NO: 10. In some embodiments, the mRNA comprises a coding sequence set forth in SEQ ID NO: 10.

In some embodiments, administering the mRNA to the subject is performed by intratracheal, intranasal, intravenous, intramuscular or subcutaneous delivery.

In some embodiments, administering the mRNA to the subject is performed by intratracheal delivery.

In some embodiments, administering the mRNA to the subject is performed by intranasal delivery.

In some embodiments, administering the mRNA to the subject is performed by aerosol delivery.

In some embodiments, administering the mRNA to the subject is performed by nebulized delivery.

In some embodiments, administering the mRNA to the subject is performed by dry powder inhalation.

In some embodiments, the composition is administered once a week.

In some embodiments, the composition is administered once every two weeks.

In some embodiments, the composition is administered twice a month.

In some embodiments, the composition is administered once a month.

In some embodiments, the composition is administered at repeat intervals. In some embodiments, the repeat intervals occur every day, 3 days, 1 week, 2 weeks, 3 weeks, or four weeks. Accordingly, in some embodiments, the repeat intervals occur every day. In some embodiments, the repeat intervals occur every 3 days. In some embodiments, the repeat intervals occur every 1 week. In some embodiments, the repeat intervals occur every 2 weeks. In some embodiments, the repeat intervals occur every 3 weeks. In some embodiments, the repeat intervals occur every 4 weeks. In some embodiments, the repeat intervals occur every 5 weeks. In some embodiments, the repeat intervals occur every 6 weeks. In some embodiments, the repeat intervals occur every 7 weeks. In some embodiments, the repeat intervals occur every 8 weeks. In some embodiments, the repeat intervals occur every 9 weeks. In some embodiments, the repeat intervals occur every 10 weeks. In some embodiments, the repeat intervals occur every 11 weeks. In some embodiments, the repeat intervals occur every 12 weeks.

In some embodiments, the administering the mRNA results in DNAI1 protein expression detectable in one or more internal organs selected from lung, heart, liver, spleen, kidney, brain, stomach, intestines, ovary and testis.

In some embodiments, the administering the mRNA results in DNAI1 protein expression detectable in the lung.

In some embodiments, the administering the mRNA results in DNAI1 protein expression detectable in the lung epithelium.

In some embodiments, the DNAI1 protein expression is detectable throughout the length of cilia. In some embodiments, the DNAI1 mRNA is detectable throughout the length of cilia.

In some aspects, the invention provides a composition for use in the treatment of primary ciliary dyskinesia (PCD), the composition comprising an mRNA encoding human axonemal dynein intermediate chain 1 (DNAI1) encapsulated in a liposome, wherein the liposome comprises one or more cationic lipids, one or more non-cationic lipids and one or more PEG-modified lipids.

In some embodiments, the mRNA comprises a DNAI1 coding sequence at least 70%, 75%, 80%, 85%, 90%, or 95% identical to any one of SEQ ID NO: 6 to SEQ ID NO: 10.

In some embodiments, the mRNA comprises a coding sequence set forth in SEQ ID NO: 6. In some embodiments, the mRNA comprises a coding sequence set forth in SEQ ID NO: 7. In some embodiments, the mRNA comprises a coding sequence set forth in SEQ ID NO: 8. In some embodiments, the mRNA comprises a coding sequence set forth in SEQ ID NO: 9. In some embodiments, the mRNA comprises a coding sequence set forth in SEQ ID NO: 10.

In some embodiments, the mRNA has a 5′-untranslated region (5′-UTR) that has a sequence set forth in SEQ ID NO: 2 or SEQ ID NO: 3, and a 3′-untranslated region (3′-UTR) that has a sequence set forth in SEQ ID NO: 4 or SEQ ID NO: 5.

In some embodiments, wherein the mRNA has one or more modified nucleotides.

In some embodiments, the modified one or more nucleotides is selected from pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and/or 2-thiocytidine.

In some embodiments, the mRNA is unmodified.

In some embodiments, the liposome is 100 nm in diameter or less.

In some embodiments, the invention provides a pharmaceutical composition comprising the composition described above and a suitable excipient.

In some aspects, the present invention provides a method of delivery of a mRNA encoding for a protein or peptide in vivo comprising administering to a subject in need of delivery a mRNA encoding a protein or peptide and having a 5′-untranslated region (5′-UTR) that has a sequence at least 70%, 75%, 80%, 85%, 90%, or 95% identical to SEQ ID NO: 2 and that is not SEQ ID NO: 3. In some embodiments, the mRNA comprises a 5′-untranslated region (5′-UTR) that has a sequence at least 70% identical to SEQ ID NO: 2 that is not SEQ ID NO: 3. In some embodiments, the mRNA comprises a 5′-untranslated region (5′-UTR) that has a sequence at least 75% identical to SEQ ID NO: 2 that is not SEQ ID NO: 3. In some embodiments, the mRNA comprises a 5′-untranslated region (5′-UTR) that has a sequence at least 80% identical to SEQ ID NO: 2 that is not SEQ ID NO: 3. In some embodiments, the mRNA comprises a 5′-untranslated region (5′-UTR) that has a sequence at least 85% identical to SEQ ID NO: 2 that is not SEQ ID NO: 3. In some embodiments, the mRNA comprises a 5′-untranslated region (5′-UTR) that has a sequence at least 90% identical to SEQ ID NO: 2 that is not SEQ ID NO: 3. In some embodiments, the mRNA comprises a 5′-untranslated region (5′-UTR) that has a sequence at least 95% identical to SEQ ID NO: 2 that is not SEQ ID NO: 3. In some embodiments, the mRNA comprises a 5′-untranslated region (5′-UTR) that is at least 96%, at least 97%, at least 98%, or at least 99% identical to SEQ ID NO: 2 that is not SEQ ID NO: 3. In some embodiments, the mRNA comprises a 5′-untranslated region (5′-UTR) set forth in SEQ ID NO: 2.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWING

The drawings are for illustration purposes only, not for limitation.

FIG. 1 is an exemplary Western blot illustrating the translation and expression of DNAI1 mRNA in HEK293T cells. Predicted molecular weight (79 kDa) of DNAI1 protein is indicated by an arrow.

FIG. 2A is an exemplary Western blot illustrating the translation and expression of codon-optimized DNAI1 mRNA in HEK293T cells. Western blot was performed with an anti-DNAI1 antibody and an anti-Vinculin antibody (loading control). FIG. 2B is an exemplary bar graphs showing DNAI1 protein expression over the vinculin protein (loading control). Construct K, which was “further codon-optimized” with higher CAI showed higher DNAI1 protein expression than the construct A, which had a CAI of 0.83. FIG. 2C is a Western blot that shows the translation and expression of additional codon-optimized DNAI1 mRNA in HEK293T cells. Western blot was performed with an anti-DNAI1 antibody and an anti-Vinculin antibody (loading control). FIG. 2D shows exemplary bar graphs showing DNAI1 protein levels from various additional codon-optimized DNAI1 mRNA expression over vinculin protein (loading control).

FIG. 3 is an exemplary Western blot illustrating in vivo delivery of hDNAI1 mRNA and expression of hDNAI1in mice lungs. Human DNAI1 protein, conjugated to a FLAG marker (DNAI1-FLAG) is distinguishable from the endogenous mouse Dnaic1, as indicated by the arrows.

FIG. 4 is an exemplary immunohistochemical (IHC) staining of lung bronchus and bronchioles 48 hours post administration of DNAI1 mRNA using an anti-DNAI1 antibody at a dilution that detects human DNAI1 protein but not mouse DNAI1 protein. An antibody dilution greater than 1:17000 was able to achieve this effect. Arrows indicate exemplary stained sites illustrating expression of DNAI1 protein.

FIG. 5A is an exemplary immunohistochemical (IHC) staining of bronchus and bronchioles of mice 48 hours post administration of DNAI1 mRNA in formulation 1. FIG. 5B is an exemplary immunohistochemical (IHC) staining of bronchus and bronchioles of mice 48 hours post administration of saline, as a negative control. Arrows indicate exemplary stained sites illustrating expression of DNAI1 protein.

FIG. 6 is an exemplary immunohistochemical (IHC) staining at higher magnification (40x). IHC staining with anti-DNAI1 antibody shows that the protein expression was detected through the entire length of cilia. Arrows indicate exemplary stained sites illustrating expression of DNAI1 protein.

FIG. 7 is an exemplary Western blot illustrating in vivo delivery of hDNAI1 mRNA and expression of hDNAI1in mice lungs, in a dose-dependent manner. Human DNAI1-FLAG protein is distinguishable from the endogenous mouse Dnaic1 by size differences, as indicated by the arrows. Vinculin, a housekeeping gene, was used as a loading control.

FIG. 8 is an exemplary Western blot illustrating in vivo delivery of hDNAI1 mRNA and expression of hDNAI1in mice lungs, at various time points. Human DNAI1protein, conjugated to a FLAG marker) is distinguishable from the endogenous mouse Dnaic1 by size differences, as indicated by the arrows. Vinculin, a housekeeping gene, was used as a loading control.

FIG. 9 is an exemplary bar graph that depicts the amount of radiance produced by luciferase protein expressed in mice after administration of mRNA-LNPs, each comprising a different cationic lipid component. The horizontal lines in the graph around 10⁴ p/s/cm²sr represent the historical radiance/expression of pulmonary-delivered (e.g., nebulized) FFL mRNA encapsulated in an LNP comprising ICE as a cationic lipid. The horizontal lines in the graph around 10⁶p/s/cm²sr represent the historical radiance/expression of pulmonary-delivered (e.g., nebulized) FFL mRNA encapsulated in an LNP comprising ML2 as a cationic lipid. These thresholds can be used to screen lipids for pulmonary delivery.

FIG. 10A is an exemplary bar graph that depicts the amount of mRNA delivered to the lung tissue as determined by RT-qPCR. FIG. 10B is an exemplary plot that depicts the amount of mCherry protein (y-axis) produced per amount of mCherry mRNA (x-axis) delivered to the lung.

FIG. 11A is an exemplary imaging of whole mice that shows radiance of firefly luciferase expressed by the delivered mRNA-LNPs. The radiance shows that mRNA-LNPs were effectively delivered to mice lungs for in vivo expression. FIG. 11B is an exemplary imaging of mice by Cryofluorescence Tomography that shows expression of Cre recombinase mRNA. The imaging shows that expression of the delivered protein is detected in the lungs and branches of the airway, as indicated by the arrows. FIG. 11C is an exemplary immunofluorescence imaging of lung sections at 40x and 100x magnification. CFTR protein expressed by the delivered mRNA was evident in apical surface of the airways, as indicated by the arrows.

FIG. 12A is an exemplary HBEC-ALI (Human Bronchial Epithelial Cell - Air Liquid Interface) model and timeline for growing the ALI culture. FIG. 12B is an exemplary imaging of the differentiated epithelium in HBEC-ALI model after staining with hematoxylin and eosin (H&E).

FIG. 13A is an exemplary bar graph that depicts the amount of luminescence produced (in log scale) by luciferase protein in cells in the HBEC-ALI model after transfection with mRNA-LNPs. FIG. 13B is an exemplary bar graph that depicts cell integrity as measured by trans-epithelial electrical resistance (TEER), which is a strong indicator of epithelium integrity.

FIG. 14 is an exemplary ROC curve (receiver operating characteristic curve) demonstrating that HBEC-ALI model shows meaningful performance as classification model for screening and filtering lipids prior to in vivo evaluation.

FIG. 15A is an exemplary graph that shows relative concentration of lipids in the HBEC-ALI model after transfection with mRNA-LNPs. The half-life determined from the graph is about 2.9 hours. FIG. 15B is an exemplary table that shows half-life values determined from mouse and human lung homogenates.

FIG. 16A shows a table summarizing immunofluorescence data acquired from mice which had received Construct A by nebulization into the lungs. FIG. 16B and FIG. 16D shows immunofluorescence images showing DNAI1 and α-tubulin staining of mouse lungs from mice which had received Construct A in Formulation G2. FIG. 16C shows immunofluorescence images showing DNAI1 and α-tubulin staining of mouse lungs from mice which had received Construct A in Formulation G3.

FIG. 17A is a graph that shows the in vivo expression of DNAI1 in lung tissue obtained from mice that had been administered Construct A. FIG. 17B shows a series of Western Blots from lung samples of mice which had received multiple doses of Construct A intratracheally for the amount of days shown. FIG. 17C is a graph that summarizes immunofluorescence results from mice which were administered Construct A in Formulation G3. FIG. 17D is a series of histological images from mice that had received Construct A for the period of time depicted in the image.

FIG. 18A is an exemplary immunohistochemical (IHC) staining of lung bronchus and bronchioles 48 hours post administration of saline, as a negative control. FIG. 18B and FIG. 18C are exemplary immunohistochemical (IHC) stainings of bronchus and bronchioles of mice 48 hours post administration of DNAI1 mRNA Construct K-1 or K2, respectively, encapsulated in LNPs using an anti-DNAI1 antibody at an optimized dilution that detects human DNAI1 protein but not mouse DNAI1 protein. Arrows indicate exemplary stained sites illustrating expression of DNAI1 protein.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Alkyl: As used herein, “alkyl” refers to a radical of a straight-chain or branched saturated hydrocarbon group having from 1 to 15 carbon atoms (“C1-15 alkyl”). In some embodiments, an alkyl group has 1 to 3 carbon atoms (“C1-3 alkyl”). Examples of C1-3 alkyl groups include methyl (C1), ethyl (C2), n-propyl (C3), and isopropyl (C3). In some embodiments, an alkyl group has 8 to 12 carbon atoms (“C8-12 alkyl”). Examples of C8-12 alkyl groups include, without limitation, n-octyl (C8), n-nonyl (C9), n-decyl (C10), n-undecyl (C11), n-dodecyl (C12) and the like. The prefix “n-” (normal) refers to unbranched alkyl groups. For example, n-C8 alkyl refers to (CH2)7CH3, n-C10 alkyl refers to (CH2)9CH3, etc.

Amino acid: As used herein, term “amino acid,” in its broadest sense, refers to any compound and/or substance that can be incorporated into a polypeptide chain. In some embodiments, an amino acid has the general structure H2N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, protecting groups, and/or substitution with other chemical groups that can change the peptide’s circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Typically, the term “approximately” or “about” refers to a range of values that within 10%, or more typically 1%, of the stated reference value.

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active.

Codon-optimized: As used herein, the term describes a nucleic acid in which one or more of the nucleotides present in a naturally occurring nucleic acid sequence (also referred to as ‘wild-type’ sequence) has been substituted with an alternative nucleotide to optimize protein expression without changing the amino acid sequence of the polypeptide encoded by the naturally occurring nucleic acid sequence. For example, the codon AAA may be altered to become AAG without changing the identity of the encoded amino acid (lysine). In some embodiments, the nucleic acids of the invention are codon optimized to increase protein expression of the protein encoded by the nucleic acid. For the purpose of this application, nucleobase thymidine (T) and uracil (U) are used interchangeably in narration of mRNA sequences.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient’s circulation system (e.g., serum) and systematically distributed and taken up by other tissues (also referred to as “systemic distribution” or “systemic delivery).

Dosing interval: As used herein dosing interval in the context of a method for treating a disease is the frequency of administering a therapeutic composition in a subject (mammal) in need thereof, for example an mRNA composition, at an effective dose of the mRNA, such that one or more symptoms associated with the disease is reduced; or one or more biomarkers associated with the disease is reduced, at least for the period of the dosing interval. Dosing frequency and dosing interval may be used interchangeably in the current disclosure.

Expression: As used herein, “expression” of a nucleic acid sequence refers to translation of an mRNA into a polypeptide, assemble multiple polypeptides into an intact protein (e.g., enzyme) and/or post-translational modification of a polypeptide or fully assembled protein (e.g., enzyme). In this application, the terms “expression” and “production,” and grammatical equivalent, are used inter-changeably.

Effective dose: As used herein, an effective dose is a dose of the mRNA in the pharmaceutical composition which when administered to the subject in need thereof, hereby a mammalian subject, according to the methods of the invention, is effective to bring about an expected outcome in the subject, for example reduce a symptom associated with the disease.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

Local distribution or delivery: As used herein, the terms “local distribution,” “local delivery,” or grammatical equivalent, refer to tissue specific delivery or distribution. Typically, local distribution or delivery requires a protein (e.g., enzyme) encoded by mRNAs be translated and expressed intracellularly or with limited secretion that avoids entering the patient’s circulation system.

messenger RNA (mRNA): As used herein, the term “messenger RNA (mRNA)” refers to a polyribonucleotide that encodes at least one polypeptide. mRNA may contain one or more coding and non-coding regions. mRNA can be purified from natural sources, produced using recombinant expression systems and optionally purified, in vitro transcribed, chemically synthesized, etc. An mRNA sequence is presented in the 5′ to 3′ direction unless otherwise indicated. Typically, the mRNA of the present invention is synthesized from adenosine, guanosine, cytidine and uridine nucleotides that bear no modifications. Such mRNA is referred to herein as mRNA with unmodified nucleotides or ‘unmodified mRNA’ for short. Typically, this means that the mRNA of the present invention does not comprise any of the following nucleoside analogs: 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine. An mRNA suitable for practising the claimed invention commonly does not comprise nucleosides comprising chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

N/P Ratio: As used herein, the term “N/P ratio” refers to a molar ratio of positively charged molecular units in the cationic lipids in a lipid nanoparticle relative to negatively charged molecular units in the mRNA encapsulated within that lipid nanoparticle. As such, N/P ratio is typically calculated as the ratio of moles of amine groups in cationic lipids in a lipid nanoparticle relative to moles of phosphate groups in mRNA encapsulated within that lipid nanoparticle.

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C1-4 alkyl)4 salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate and aryl sulfonate. Further pharmaceutically acceptable salts include salts formed from the quarternization of an amine using an appropriate electrophile, e.g., an alkyl halide, to form a quarternized alkylated amino salt.

Systemic distribution or delivery: As used herein, the terms “systemic distribution,” “systemic delivery,” or grammatical equivalent, refer to a delivery or distribution mechanism or approach that affect the entire body or an entire organism. Typically, systemic distribution or delivery is accomplished via body’s circulation system, e.g., blood stream. Compared to the definition of “local distribution or delivery.”

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Various aspects of the invention are described in detail in the following sections. The use of sections is not meant to limit the invention. Each section can apply to any aspect of the invention. In this application, the use of “or” means “and/or” unless stated otherwise.

### DETAILED DESCRIPTION

### Primary Ciliary Dyskinesia (PCD)

Primary ciliary dyskinesia (PCD) is an autosomal recessive disorder characterized by abnormal cilia and flagella that are found in the linings of the airway, the reproductive system, and other organs and tissues. The main ciliary defect found in PCD is an absence of dynein arms, affecting almost all cilia.

Dyneins consist of a large family of proteins involved in many types of microtubule-dependent cell motility in both lower and higher eukaryotes. Two major classes of dyneins have been described: cytoplasmic and axonemal dyneins. Cytoplasmic dyneins are involved in a wide range of intracellular functions, including chromosome movements on the mitotic spindle and the transport of membranous organelles toward the minus ends of microtubules. Axonemal dyneins are found in ciliary and flagellar axonemes. Two dynein arms—outer and inner—are bound to each peripheral microtubule doublet; these dynein arms are essential for ciliary and flagellar beating, which they generate through ATP-dependent cycles of attachment/detachment to the adjacent microtubule doublet.

Among several genes confirmed to be directly involved in PCD pathogenesis, a significant number of mutations were found in two genes: DNAI1 and DNAH5, encoding intermediate and heavy chains of the axonemal dynein, respectively. Mutations in other genes, coding for proteins involved in the axonemal ultrastructure (DNAH11, DNAI2, TXNDC3, RSPH9, RSPH4A) or assembly (KTU, CRRC50), also have been reported, as well as mutations in the RPGR gene, in some cases of PCD. Mutations in DNAI1 and DNAH5, both associated with a ciliary outer dynein arm (ODA) defect phenotype, are combined estimated to account for almost 40% of PCD cases.

Mutations in the DNAI1 gene result in an absent or abnormal dynein axonemal intermediate chain 1, which is required for the proper functioning of cilia. Without a normal version of DNAI1, defective cilia cannot produce the force and movement needed to eliminate fluid, bacteria, and particles from the lungs, to establish the left-right axis during embryonic development, and to propel the sperm cells. PCD can lead to chronic respiratory tract infections, bronchiectasis, year-round nasal congestion, abnormally placed organs within their chest and abdomen, and infertility.

Polyribonucleotides of the disclosure can be used, for example, to treat a subject having or at risk of having primary ciliary dyskinesia or any other condition associated with a defect or malfunction of a gene whose function is linked to cilia maintenance and function. Non limiting examples of genes that have been associated with primary ciliary dyskinesia include: armadillo repeat containing 4 (ARMC4), chromosome 21 open reading frame 59 (C21orf59), coiled-coil domain containing 103 (CCDC103), coiled-coil domain containing 114 (CCDC114), coiled-coil domain containing 39 (CCDC39), coiled-coil domain containing 40 (CCDC40), coiled-coil domain containing 65 (CCDC65), cyclin O (CCNO), dynein (axonemal) assembly factor 1 (DNAAF1), dynein (axonemal) assembly factor 2 (DNAAF2), dynein (axonemal) assembly factor 3 (DNAAF3), dynein (axonemal) assembly factor 5 (DNAAF5), dynein axonemal heavy chain 11 (DNAH11), dynein axonemal heavy chain 5 (DNAH5), dynein axonemal heavy chain 6 (DNAH6),dynein axonemal heavy chain 8 (DNAH8), dynein axonemal intermediate chain 1 (DNAI1), dynein axonemal intermediate chain 2 (DNAI2), dynein axonemal light chain 1 (DNAL1), dynein regulatory complex subunit 1 (DRC1), dyslexia susceptibility 1 candidate 1 (DYX1C1), growth arrest specific 8 (GAS8), axonemal central pair apparatus protein (HYDIN), leucine rich repeat containing 6 (LRRC6), ME/ M23 family member 8 (NME8), oral-facial-digital syndrome 1 (OFD1), retinitis pigmentosa GTPase regulator (RPGR), radial spoke head 1 homolog (Chlamydomonas) (RSPH1), radial spoke head 4 homolog A (Chlamydomonas) (RSPH4A), radial spoke head 9 homolog (Chlamydomonas) (RSPH9), sperm associated antigen 1(SPAG1), and zinc finger MY D-type containing 10 (ZMYND10).

### Dynein Axonemal Intermediate Chain 1 (DNAI1) Gene and Protein Sequence

In some embodiments, the present invention provides methods and compositions for delivering mRNA encoding to a subject for the treatment of PCD. A suitable DNAI1 mRNA encodes any full length, fragment or portion of a DNAI1 protein which can be substituted for naturally-occurring DNAI1 protein activity and/or reduce the intensity, severity, and/or frequency of one or more symptoms associated with PCD.

In some embodiments, a suitable mRNA sequence is an mRNA sequence encoding a human DNAI1 protein. The naturally-occurring human DNAI1 mRNA coding sequence and the corresponding amino acid sequence are shown in Table 1:

TABLE 1

Human DNAI1 Amino Acid Sequence

| | |
|---|---|
| Human DNAI1 Protein Sequence | MIPASAKAPHKQPHKQSISIGRGTRKRDEDSGTEVGEGTDEWAQSKATVRP PDQLELTDAELKEEFTRILTANNPHAPQNIVRYSFKEGTYKPIGFVNQLAVH YTQVGNLIPKDSDEGRRQHYRDELVAGSQESVKVISETGNLEEDEEPKELET EPGSQTDVPAAGAAEKVTEEELMTPKQPKERKLTNQFNFSERASQTYNNPV RDRECQTEPPPRTNFSATANQWEIYDAYVEELEKQEKTKEKEKAKTPVAKK SGKMAMRKLTSMESQTDDLIKLSQAAKIMERMVNQNTYDDIAQDFKYYDD AADEYRDQVGTLLPLWKFQNDKAKRLSVTALCWNPKYRDLFAVGYGSYD FMKQSRGMLLLYSLKNPSFPEYMFSSNSGVMCLDIHVDHPYLVAVGHYDG NVAIYNLKKPHSQPSFCSSAKSGKHSDPVWQVKWQKDDMDQNLNFFSVSS DGRIVSWTLVKRKLVHIDVIKLKVEGSTTEVPEGLQLHPVGCGTAFDFHKEI DYMFLVGTEEGKIYKCSKSYSSQFLDTYDAHNMSVDTVSWNPYHTKVFMS CSSDWTVKIWDHTIKTPMFIYDLNSAVGDVAWAPYSSTVFAAVTTDGKAHI FDLAINKYEAICNQPVAAKKNRLTHVQFNLIHPIIIVGDDRGHIISLKLSPNLR KMPKEKKGQEVQKGPAVEIAKLDKLLNLVREVKIKT (SEQ ID NO: 1) |

Various mRNA sequences used in the Example section or useful for the present invention are shown in Table 2:

TABLE 2

Various DNAI1 Nucleic Acid Sequences

| | |
|---|---|
| DNAI1-FLAG (Human) | ATGATCCCCGCCTCCGCCAAAGCCCCTCACAAGCAACCGCACAAGCAAA GCATTAGCATTGGGCGGGGTACTCGGAAGCGCGACGAGGACTCGGGAA CTGAAGTCGGAGAGGGGACCGACGAATGGGCGCAGTCAAAGGCCACCG TGCGCCCACCGGACCAGCTCGAGCTGACCGATGCTGAGCTGAAGGAGGA GTTTACCCGGATCCTGACAGCCAACAACCCACATGCACCGCAGAACATC GTGCGGTACAGCTTCAAAGAGGGAACTTATAAGCCCATTGGCTTCGTGA ACCAACTCGCGGTGCATTACACCCAAGTCGGAAACCTTATTCCGAAGGA CTCGGACGAAGGCAGACGCCAGCACTACCGGGACGAGCTCGTGGCAGG ATCCCAGGAAAGCGTCAAGGTCATTTCCGAGACTGGCAACCTCGAGGAG GACGAAGAACCTAAGGAGCTGGAAACCGAACCCGGATCCCAGACCGAC GTGCCGGCCGCTGGGGCTGCCGAGAAAGTCACTGAAGAGGAACTCATGA CCCCGAAGCAGCCGAAAGAGAGAAAGCTCACCAACCAATTCAACTTCAG CGAGCGCGCCAGCCAAACCTACAACAACCCAGTCAGGGATCGGGAATGT CAGACCGAACCGCCTCCGAGAACGAACTTCTCGGCGACCGCGAACCAAT |
| | GGGAGATCTACGACGCCTACGTGGAAGAACTGGAAAAGCAGGAAAAGA CTAAGGAAAAGGAAAAGGCCAAGACTCCCGTCGCCAAGAAGTCGGGCA AAATGGCCATGCGGAAGCTCACCTCCATGGAATCACAGACTGACGACTT GATCAAGTTGAGCCAGGCCGCAAAGATCATGGAGCGCATGGTCAACCAA AATACTTACGACGATATCGCCCAAGACTTCAAGTACTACGACGACGCTG CCGATGAATACCGAGATCAAGTCGGCACCCTACTGCCGCTTTGGAAGTT CCAGAATGACAAGGCCAAGAGGCTGAGCGTGACCGCGCTGTGCTGGAA CCCCAAATACCGCGACCTCTTCGCCGTGGGATACGGCTCCTACGATTTCA TGAAGCAGAGCCGGGGAATGTTGCTCCTTTACTCCCTGAAGAACCCCTC CTTCCCTGAGTACATGTTCAGCTCAAACAGCGGCGTGATGTGCCTCGACA TTCACGTGGACCACCCTTACCTCGTGGCCGTGGGTCACTACGACGGCAA CGTCGCGATCTACAACTTGAAGAAGCCGCATTCACAGCCCTCGTTTTGCT CCTCGGCCAAGTCCGGCAAACATTCGGACCCAGTGTGGCAAGTCAAGTG GCAGAAAGATGACATGGACCAAAACTTGAACTTCTTCAGCGTGTCCTCC GACGGACGGATCGTGTCCTGGACCCTCGTGAAGCGGAAGTTGGTGCATA TCGACGTGATCAAATTGAAGGTCGAGGGTTCGACCACCGAAGTGCCTGA AGGCCTGCAGCTTCACCCCGTGGGATGCGGCACTGCCTTCGACTTCCACA AGGAGATCGACTACATGTTCCTCGTGGGAACCGAGGAAGGGAAGATCTA CAAATGCAGCAAGTCCTACTCATCACAATTCCTGGATACCTACGATGCCC ACAACATGAGCGTGGATACCGTGTCGTGGAACCCCTATCACACCAAGGT ATTCATGTCCTGCTCCTCCGACTGGACCGTCAAGATTTGGGACCACACCA TCAAGACCCCCATGTTCATCTACGACCTGAACTCCGCCGTGGGGGATGT GGCCTGGGCCCCCTACTCGTCGACCGTGTTTGCCGCGGTCACCACGGAC GGAAAGGCACACATTTTCGACCTTGCGATTAACAAATACGAGGCGATTT GCAACCAGCCCGTGGCCGCCAAAAAGAACCGCCTGACCCACGTTCAATT CAACTTAATCCACCCAATCATCATCGTCGGCGATGACAGAGGACACATT ATTAGCCTGAAACTTAGCCCCAACCTCCGCAAGATGCCCAAGGAGAAGA AGGGACAGGAAGTCCAGAAGGGCCCTGCCGTGGAGATTGCAAAGCTCG ATAAGCTCCTGAACTTAGTCCGGGAAGTGAAGATCAAGACTGGGGGCGG ATCCGACTATAAGGATGATGACGATAAGTAA (SEQ ID NO: 12) |
| DNAI1-HA (Human) | ATGATCCCCGCCTCCGCCAAAGCCCCTCACAAGCAACCGCACAAGCAAA GCATTAGCATTGGGCGGGGTACTCGGAAGCGCGACGAGGACTCGGGAA CTGAAGTCGGAGAGGGGACCGACGAATGGGCGCAGTCAAAGGCCACCG TGCGCCCACCGGACCAGCTCGAGCTGACCGATGCTGAGCTGAAGGAGGA GTTTACCCGGATCCTGACAGCCAACAACCCACATGCACCGCAGAACATC GTGCGGTACAGCTTCAAAGAGGGAACTTATAAGCCCATTGGCTTCGTGA ACCAACTCGCGGTGCATTACACCCAAGTCGGAAACCTTATTCCGAAGGA CTCGGACGAAGGCAGACGCCAGCACTACCGGGACGAGCTCGTGGCAGG ATCCCAGGAAAGCGTCAAGGTCATTTCCGAGACTGGCAACCTCGAGGAG GACGAAGAACCTAAGGAGCTGGAAACCGAACCCGGATCCCAGACCGAC GTGCCGGCCGCTGGGGCTGCCGAGAAAGTCACTGAAGAGGAACTCATGA |
| | CCCCGAAGCAGCCGAAAGAGAGAAAGCTCACCAACCAATTCAACTTCAG CGAGCGCGCCAGCCAAACCTACAACAACCCAGTCAGGGATCGGGAATGT CAGACCGAACCGCCTCCGAGAACGAACTTCTCGGCGACCGCGAACCAAT GGGAGATCTACGACGCCTACGTGGAAGAACTGGAAAAGCAGGAAAAGA CTAAGGAAAAGGAAAAGGCCAAGACTCCCGTCGCCAAGAAGTCGGGCA AAATGGCCATGCGGAAGCTCACCTCCATGGAATCACAGACTGACGACTT GATCAAGTTGAGCCAGGCCGCAAAGATCATGGAGCGCATGGTCAACCAA AATACTTACGACGATATCGCCCAAGACTTCAAGTACTACGACGACGCTG CCGATGAATACCGAGATCAAGTCGGCACCCTACTGCCGCTTTGGAAGTT CCAGAATGACAAGGCCAAGAGGCTGAGCGTGACCGCGCTGTGCTGGAA CCCCAAATACCGCGACCTCTTCGCCGTGGGATACGGCTCCTACGATTTCA TGAAGCAGAGCCGGGGAATGTTGCTCCTTTACTCCCTGAAGAACCCCTC CTTCCCTGAGTACATGTTCAGCTCAAACAGCGGCGTGATGTGCCTCGACA TTCACGTGGACCACCCTTACCTCGTGGCCGTGGGTCACTACGACGGCAA CGTCGCGATCTACAACTTGAAGAAGCCGCATTCACAGCCCTCGTTTTGCT CCTCGGCCAAGTCCGGCAAACATTCGGACCCAGTGTGGCAAGTCAAGTG GCAGAAAGATGACATGGACCAAAACTTGAACTTCTTCAGCGTGTCCTCC GACGGACGGATCGTGTCCTGGACCCTCGTGAAGCGGAAGTTGGTGCATA TCGACGTGATCAAATTGAAGGTCGAGGGTTCGACCACCGAAGTGCCTGA AGGCCTGCAGCTTCACCCCGTGGGATGCGGCACTGCCTTCGACTTCCACA AGGAGATCGACTACATGTTCCTCGTGGGAACCGAGGAAGGGAAGATCTA CAAATGCAGCAAGTCCTACTCATCACAATTCCTGGATACCTACGATGCCC ACAACATGAGCGTGGATACCGTGTCGTGGAACCCCTATCACACCAAGGT ATTCATGTCCTGCTCCTCCGACTGGACCGTCAAGATTTGGGACCACACCA TCAAGACCCCCATGTTCATCTACGACCTGAACTCCGCCGTGGGGGATGT GGCCTGGGCCCCCTACTCGTCGACCGTGTTTGCCGCGGTCACCACGGAC GGAAAGGCACACATTTTCGACCTTGCGATTAACAAATACGAGGCGATTT GCAACCAGCCCGTGGCCGCCAAAAAGAACCGCCTGACCCACGTTCAATT CAACTTAATCCACCCAATCATCATCGTCGGCGATGACAGAGGACACATT ATTAGCCTGAAACTTAGCCCCAACCTCCGCAAGATGCCCAAGGAGAAGA AGGGACAGGAAGTCCAGAAGGGCCCTGCCGTGGAGATTGCAAAGCTCG ATAAGCTCCTGAACTTAGTCCGGGAAGTGAAGATCAAGACTGGGGGCGG ATCCTACCCCTACGACGTGCCAGACTACGCATAA (SEQ ID NO: 13) |
| DNAI1-stop (Human) | ATGATCCCCGCCTAAGCCAAAGCCCCTCATAAGCAACCGCACAAGCAAA GCATAAGCTGAGGGCGGGGTACTCGGAAGCGTGACGAGGACTCGGGAA CTGAAGTAGGAGAGGGGACCGACGAATGGGCGCAGTCAAAGGCCACCG TGCGCCCACCGGACCAGCTCGAGCTGACCGATGCTGAGCTGAAGGAGGA GTTTACCCGGATCCTGACAGCCAACAACCCACATGCACCGCAGAACATC GTGCGGTACAGCTTCAAAGAGGGAACTTATAAGCCCATTGGCTTCGTGA ACCAACTCGCGGTGCATTACACCCAAGTCGGAAACCTTATTCCGAAGGA CTCGGACGAAGGCAGACGCCAGCACTACCGGGACGAGCTCGTGGCAGG |
| | ATCCCAGGAAAGCGTCAAGGTCATTTCCGAGACTGGCAACCTCGAGGAG GACGAAGAACCTAAGGAGCTGGAAACCGAACCCGGATCCCAGACCGAC GTGCCGGCCGCTGGGGCTGCCGAGAAAGTCACTGAAGAGGAACTCATGA CCCCGAAGCAGCCGAAAGAGAGAAAGCTCACCAACCAATTCAACTTCAG CGAGCGCGCCAGCCAAACCTACAACAACCCAGTCAGGGATCGGGAATGT CAGACCGAACCGCCTCCGAGAACGAACTTCTCGGCGACCGCGAACCAAT GGGAGATCTACGACGCCTACGTGGAAGAACTGGAAAAGCAGGAAAAGA CTAAGGAAAAGGAAAAGGCCAAGACTCCCGTCGCCAAGAAGTCGGGCA AAATGGCCATGCGGAAGCTCACCTCCATGGAATCACAGACTGACGACTT GATCAAGTTGAGCCAGGCCGCAAAGATCATGGAGCGCATGGTCAACCAA AATACTTACGACGATATCGCCCAAGACTTCAAGTACTACGACGACGCTG CCGATGAATACCGAGATCAAGTCGGCACCCTACTGCCGCTTTGGAAGTT CCAGAATGACAAGGCCAAGAGGCTGAGCGTGACCGCGCTGTGCTGGAA CCCCAAATACCGCGACCTCTTCGCCGTGGGATACGGCTCCTACGATTTCA TGAAGCAGAGCCGGGGAATGTTGCTCCTTTACTCCCTGAAGAACCCCTC CTTCCCTGAGTACATGTTCAGCTCAAACAGCGGCGTGATGTGCCTCGACA TTCACGTGGACCACCCTTACCTCGTGGCCGTGGGTCACTACGACGGCAA CGTCGCGATCTACAACTTGAAGAAGCCGCATTCACAGCCCTCGTTTTGCT CCTCGGCCAAGTCCGGCAAACATTCGGACCCAGTGTGGCAAGTCAAGTG GCAGAAAGATGACATGGACCAAAACTTGAACTTCTTCAGCGTGTCCTCC GACGGACGGATCGTGTCCTGGACCCTCGTGAAGCGGAAGTTGGTGCATA TCGACGTGATCAAATTGAAGGTCGAGGGTTCGACCACCGAAGTGCCTGA AGGCCTGCAGCTTCACCCCGTGGGATGCGGCACTGCCTTCGACTTCCACA AGGAGATCGACTACATGTTCCTCGTGGGAACCGAGGAAGGGAAGATCTA CAAATGCAGCAAGTCCTACTCATCACAATTCCTGGATACCTACGATGCCC ACAACATGAGCGTGGATACCGTGTCGTGGAACCCCTATCACACCAAGGT ATTCATGTCCTGCTCCTCCGACTGGACCGTCAAGATTTGGGACCACACCA TCAAGACCCCCATGTTCATCTACGACCTGAACTCCGCCGTGGGGGATGT GGCCTGGGCCCCCTACTCGTCGACCGTGTTTGCCGCGGTCACCACGGAC GGAAAGGCACACATTTTCGACCTTGCGATTAACAAATACGAGGCGATTT GCAACCAGCCCGTGGCCGCCAAAAAGAACCGCCTGACCCACGTTCAATT CAACTTAATCCACCCAATCATCATCGTCGGCGATGACAGAGGACACATT ATTAGCCTGAAACTTAGCCCCAACCTCCGCAAGATGCCCAAGGAGAAGA AGGGACAGGAAGTCCAGAAGGGCCCTGCCGTGGAGATTGCAAAGCTCG ATAAGCTCCTGAACTTAGTCCGGGAAGTGAAGATCAAGACTTAA (SEQ ID NO: 14) |
| mDnaic 1(Mouse) | ATGCCATCCAAACAAATTAGAAAGCAGAGCATTAGCGTGACCCGCGGAG CCCGGAGAAGAGATGAGGACTCGGGCACCGATGTCGGCGAAGGAACCG ACGAGTGGTCGCAGTCAAAGGCTACCGTGCGGCCGCCTGACCAGCTTGA ACTTACCGATGCCGAGCTGAAGGAAGAGTTCACTCGCATCCTGACCGCC AACAATCCCCACGCGCCGCAGAACATCGTGCGCTACTCCTTCAAAGAAG |
| | GAACGTACAAGCTCATTGGATTCGTGAACCAAATGGCCGTGCACTTTTCC CAAGTCGGAAACCTCATTCCGAAGGATTCCGACGAGGGCCGGCGGCAGC ATTACCGCGACGAAATGGTGGCCGGATCGCAAGAAAGCATCAAGGTCGT GACTTCCGAGGCCGAGAACCTCGAGGAAGAAGAAGAACCGAAAGAGGG CGAAGGAGAAGCCGAAGCCGAGGCTGAGGCCGGAAGCCAGACCGATAT CCCCGCTGCCGCAGAAACTACCGAAAAGGTCATCGAAGAGGAGTTGATG GCCCCGGTGCAGCCTAAGGAACGGAAGTTGACTAACCAATTCAACTTCA GCGAGAGAGCCTCACAGACCTTCAACAACCCACTGAGAGATAGGGAAT GTCAAATGGAGCCTCCGCCCCGGACGAACTTCTCGGCCACTGCAAACCA ATGGGAGATCTACGACGCTTACGTGGATGAACTGGAGAAGCAGGAAAA GACCAAGGAAAAGGAGAAAGCCAAGACCCCGGTCGCCAAAAAGACTGA GAAAATGGCCATGCGGAAGCTCACTTCCATGGAGAGCCAGTCCGACGAT ATTACTAAGGTTACCCAGGCCGCCAAGATCGTGGAAAGAATGGTGAACC AGAATACCTACGACGACGTGGCACAGGATTTTAAGTACTATGAGGACAC CGCTGACGAATACCGGGATCAAGAAGGCACCCTCCTGCCCTTGTGGAAG TTCCAGAATGACAAGGCCAAGAGGCTGGCCGTGACTGCGCTGTGCTGGA ACCCGAAGTATAAGGACCTCTTCGCCGTGGGTCATGGCTCATACGACTTT ATGAAGCAATCGCGCGGGATGCTCCTGCTCTACTCGATGAAGAACCCTT CCTTCCCCGAGTACATGTTCAGCTCCGAGTCAGGGATCATGTGCCTGGAT GTCCATGTCGACCACCCTTACCTCGTGGTCGTGGGATATTACGACGGCAA CGTGGCGATCTACAACCTTAAGAAACCGCATAGCCAGCCCTGCTTCCGG AGCACCTCAAAGAGCGGAAAGCACACCGACCCCGTGTGGCAAGTCAAG TGGCAAAAGGACGACATGGACCACAACTTGAACTTCTTCAGCGTGTCGT CCGACGGACGGATCGTGTCCTGGACCCTCGTCAAGTCCGAGCTCGTGCA CATTGACATTATCAAGCTCAAAACCGAGGGTTCCACCACTGAAATCCCC GAGGGCCTGCAGCTCCATACCGTGGGATGCGGCACCGCGTTCGACTTCC ATAAAGAGATCGACTACATGTTCCTCGTGGGGACTGAAGAGGGGAAGAT CTACAAGTGCAGCAAGTCGTACTCTTCCCAATTCCTGGACACCTACGACG CTCATAACATGGCAGTGGACGCCGTGCTGTGGAACCCATACCACACTAA GGTCTTTATGTCCTGCTCCTCGGACTGGACTGTGAAGATTTGGGACCACA CCATCAAAACCCCAATGTTCATTTACGACCTGAACTCCGCCGTGGGGGA TGTGGCCTGGGCCCCGTATTCCTCCACCGTATTCGCCGCCGTGACCACCG ACGGAAAGGCACACGTCTTTGACCTGGCCGTGAACAAATACGAGGCGAT TTGCAACCAGCCAGTGGTGGCCAAGAAGAAAAACAAGATCACTCACGTG CAATTCAACCCCATCCACCCCATTATTATCGTCGGCGACGATAGGGGTCA CATTATATGCCTGAAGCTCAGCCCGAACCTTCGCAAGATGCCTAAGGAA AAGAAGGGCCAGGAGGTGCAGAAGGGTCCTGCCGTCGAAATTGCGAAG TTGGACAAGCTCCTGAACCTCGTGCGCGAGGTCAAGACTAAGACCTAA (SEQ ID NO: 11) |
| mDnaic1-FLAG (Mouse) | ATGCCATCCAAACAAATTAGAAAGCAGAGCATTAGCGTGACCCGCGGAG CCCGGAGAAGAGATGAGGACTCGGGCACCGATGTCGGCGAAGGAACCG ACGAGTGGTCGCAGTCAAAGGCTACCGTGCGGCCGCCTGACCAGCTTGA ACTTACCGATGCCGAGCTGAAGGAAGAGTTCACTCGCATCCTGACCGCC AACAATCCCCACGCGCCGCAGAACATCGTGCGCTACTCCTTCAAAGAAG GAACGTACAAGCTCATTGGATTCGTGAACCAAATGGCCGTGCACTTTTCC CAAGTCGGAAACCTCATTCCGAAGGATTCCGACGAGGGCCGGCGGCAGC ATTACCGCGACGAAATGGTGGCCGGATCGCAAGAAAGCATCAAGGTCGT GACTTCCGAGGCCGAGAACCTCGAGGAAGAAGAAGAACCGAAAGAGGG CGAAGGAGAAGCCGAAGCCGAGGCTGAGGCCGGAAGCCAGACCGATAT CCCCGCTGCCGCAGAAACTACCGAAAAGGTCATCGAAGAGGAGTTGATG GCCCCGGTGCAGCCTAAGGAACGGAAGTTGACTAACCAATTCAACTTCA GCGAGAGAGCCTCACAGACCTTCAACAACCCACTGAGAGATAGGGAAT GTCAAATGGAGCCTCCGCCCCGGACGAACTTCTCGGCCACTGCAAACCA ATGGGAGATCTACGACGCTTACGTGGATGAACTGGAGAAGCAGGAAAA GACCAAGGAAAAGGAGAAAGCCAAGACCCCGGTCGCCAAAAAGACTGA GAAAATGGCCATGCGGAAGCTCACTTCCATGGAGAGCCAGTCCGACGAT ATTACTAAGGTTACCCAGGCCGCCAAGATCGTGGAAAGAATGGTGAACC AGAATACCTACGACGACGTGGCACAGGATTTTAAGTACTATGAGGACAC CGCTGACGAATACCGGGATCAAGAAGGCACCCTCCTGCCCTTGTGGAAG TTCCAGAATGACAAGGCCAAGAGGCTGGCCGTGACTGCGCTGTGCTGGA ACCCGAAGTATAAGGACCTCTTCGCCGTGGGTCATGGCTCATACGACTTT ATGAAGCAATCGCGCGGGATGCTCCTGCTCTACTCGATGAAGAACCCTT CCTTCCCCGAGTACATGTTCAGCTCCGAGTCAGGGATCATGTGCCTGGAT GTCCATGTCGACCACCCTTACCTCGTGGTCGTGGGATATTACGACGGCAA CGTGGCGATCTACAACCTTAAGAAACCGCATAGCCAGCCCTGCTTCCGG AGCACCTCAAAGAGCGGAAAGCACACCGACCCCGTGTGGCAAGTCAAG TGGCAAAAGGACGACATGGACCACAACTTGAACTTCTTCAGCGTGTCGT CCGACGGACGGATCGTGTCCTGGACCCTCGTCAAGTCCGAGCTCGTGCA CATTGACATTATCAAGCTCAAAACCGAGGGTTCCACCACTGAAATCCCC GAGGGCCTGCAGCTCCATACCGTGGGATGCGGCACCGCGTTCGACTTCC ATAAAGAGATCGACTACATGTTCCTCGTGGGGACTGAAGAGGGGAAGAT CTACAAGTGCAGCAAGTCGTACTCTTCCCAATTCCTGGACACCTACGACG CTCATAACATGGCAGTGGACGCCGTGCTGTGGAACCCATACCACACTAA GGTCTTTATGTCCTGCTCCTCGGACTGGACTGTGAAGATTTGGGACCACA CCATCAAAACCCCAATGTTCATTTACGACCTGAACTCCGCCGTGGGGGA TGTGGCCTGGGCCCCGTATTCCTCCACCGTATTCGCCGCCGTGACCACCG ACGGAAAGGCACACGTCTTTGACCTGGCCGTGAACAAATACGAGGCGAT TTGCAACCAGCCAGTGGTGGCCAAGAAGAAAAACAAGATCACTCACGTG CAATTCAACCCCATCCACCCCATTATTATCGTCGGCGACGATAGGGGTCA CATTATATGCCTGAAGCTCAGCCCGAACCTTCGCAAGATGCCTAAGGAA AAGAAGGGCCAGGAGGTGCAGAAGGGTCCTGCCGTCGAAATTGCGAAG |
| | TTGGACAAGCTCCTGAACCTCGTGCGCGAGGTCAAGACTAAGACCGGGG GTGGTTCCGACTACAAGGATGATGACGATAA (SEQ ID NO: 15) |
| mDnaic1-HA (Mouse) | ATGCCATCCAAACAAATTAGAAAGCAGAGCATTAGCGTGACCCGCGGAG CCCGGAGAAGAGATGAGGACTCGGGCACCGATGTCGGCGAAGGAACCG ACGAGTGGTCGCAGTCAAAGGCTACCGTGCGGCCGCCTGACCAGCTTGA ACTTACCGATGCCGAGCTGAAGGAAGAGTTCACTCGCATCCTGACCGCC AACAATCCCCACGCGCCGCAGAACATCGTGCGCTACTCCTTCAAAGAAG GAACGTACAAGCTCATTGGATTCGTGAACCAAATGGCCGTGCACTTTTCC CAAGTCGGAAACCTCATTCCGAAGGATTCCGACGAGGGCCGGCGGCAGC ATTACCGCGACGAAATGGTGGCCGGATCGCAAGAAAGCATCAAGGTCGT GACTTCCGAGGCCGAGAACCTCGAGGAAGAAGAAGAACCGAAAGAGGG CGAAGGAGAAGCCGAAGCCGAGGCTGAGGCCGGAAGCCAGACCGATAT CCCCGCTGCCGCAGAAACTACCGAAAAGGTCATCGAAGAGGAGTTGATG GCCCCGGTGCAGCCTAAGGAACGGAAGTTGACTAACCAATTCAACTTCA GCGAGAGAGCCTCACAGACCTTCAACAACCCACTGAGAGATAGGGAAT GTCAAATGGAGCCTCCGCCCCGGACGAACTTCTCGGCCACTGCAAACCA ATGGGAGATCTACGACGCTTACGTGGATGAACTGGAGAAGCAGGAAAA GACCAAGGAAAAGGAGAAAGCCAAGACCCCGGTCGCCAAAAAGACTGA GAAAATGGCCATGCGGAAGCTCACTTCCATGGAGAGCCAGTCCGACGAT ATTACTAAGGTTACCCAGGCCGCCAAGATCGTGGAAAGAATGGTGAACC AGAATACCTACGACGACGTGGCACAGGATTTTAAGTACTATGAGGACAC CGCTGACGAATACCGGGATCAAGAAGGCACCCTCCTGCCCTTGTGGAAG TTCCAGAATGACAAGGCCAAGAGGCTGGCCGTGACTGCGCTGTGCTGGA ACCCGAAGTATAAGGACCTCTTCGCCGTGGGTCATGGCTCATACGACTTT ATGAAGCAATCGCGCGGGATGCTCCTGCTCTACTCGATGAAGAACCCTT CCTTCCCCGAGTACATGTTCAGCTCCGAGTCAGGGATCATGTGCCTGGAT GTCCATGTCGACCACCCTTACCTCGTGGTCGTGGGATATTACGACGGCAA CGTGGCGATCTACAACCTTAAGAAACCGCATAGCCAGCCCTGCTTCCGG AGCACCTCAAAGAGCGGAAAGCACACCGACCCCGTGTGGCAAGTCAAG TGGCAAAAGGACGACATGGACCACAACTTGAACTTCTTCAGCGTGTCGT CCGACGGACGGATCGTGTCCTGGACCCTCGTCAAGTCCGAGCTCGTGCA CATTGACATTATCAAGCTCAAAACCGAGGGTTCCACCACTGAAATCCCC GAGGGCCTGCAGCTCCATACCGTGGGATGCGGCACCGCGTTCGACTTCC ATAAAGAGATCGACTACATGTTCCTCGTGGGGACTGAAGAGGGGAAGAT CTACAAGTGCAGCAAGTCGTACTCTTCCCAATTCCTGGACACCTACGACG CTCATAACATGGCAGTGGACGCCGTGCTGTGGAACCCATACCACACTAA GGTCTTTATGTCCTGCTCCTCGGACTGGACTGTGAAGATTTGGGACCACA CCATCAAAACCCCAATGTTCATTTACGACCTGAACTCCGCCGTGGGGGA TGTGGCCTGGGCCCCGTATTCCTCCACCGTATTCGCCGCCGTGACCACCG ACGGAAAGGCACACGTCTTTGACCTGGCCGTGAACAAATACGAGGCGAT TTGCAACCAGCCAGTGGTGGCCAAGAAGAAAAACAAGATCACTCACGTG |
| | CAATTCAACCCCATCCACCCCATTATTATCGTCGGCGACGATAGGGGTCA CATTATATGCCTGAAGCTCAGCCCGAACCTTCGCAAGATGCCTAAGGAA AAGAAGGGCCAGGAGGTGCAGAAGGGTCCTGCCGTCGAAATTGCGAAG TTGGACAAGCTCCTGAACCTCGTGCGCGAGGTCAAGACTAAGACCGGGG GTGGTTCCTACCCATACGATGTGCCTGACTACGCGTA (SEQ ID NO: 16) |

In some embodiments, a suitable mRNA is a wild-type human DNAI1 mRNA of sequence. In some embodiments, a suitable therapeutic candidate mRNA is a codon-optimized DNAI1 sequence that can encodes a DNAI1 amino acid sequence shown in Table 1 as SEQ ID NO: 1 or an amino acid sequence at least 70%, 75%, 80%, 85%, 90%, 95%, 96%, 97%, 98%, or 99% identical to SEQ ID NO: 1. In some embodiments, an mRNA according to the present invention encodes a DNAI1 protein with an amino acid sequence that is identical to SEQ ID NO: 1.

### Codon Optimization

According to an increasing amount of research, mRNAs contain numerous layers of information that overlap the amino acid code. Traditionally, codon optimization has been used to remove rare codons which were thought to be rate-limiting for protein expression. While fast growing bacteria and yeast both exhibit strong codon bias in highly expressed genes, higher eukaryotes exhibit much less codon bias, making it more difficult to discern codons that may be rate-limiting. In addition, it has been found that codon bias per se does not necessarily yield high expression but requires other features.

For example, rare codons have been implicated in slowing translation and forming pause sites, which may be required for correct protein folding. Therefore, variations in codon usage may provide a mechanism to fine-tune the temporal pattern of elongation and thus increase the time available for a protein to take on its correct confirmation. Codon optimization can interfere with this fine-tuning mechanism, resulting in less efficient protein translation or an increased amount of incorrectly folded proteins. Similarly, codon optimization may disrupt the normal patterns of cognate and wobble tRNA usage, thereby affecting protein structure and function because wobble-dependent slowing of elongation may likewise have been selected as a mechanism for achieving correct protein folding.

Various methods of performing codon optimization are known in the art, however, each has significant drawbacks and limitations from a computational and/or therapeutic point of view. In particular, known methods of codon optimization often involve, for each amino acid, replacing every codon with the codon having the highest usage for that amino acid, such that the “optimized” sequence contains only one codon encoding each amino acid (so may be referred to as a one-to-one sequence).

Despite these obstacles, the inventors have arrived at a codon-optimized hDNAI1sequence that improves expression of the DNAI1 protein at least threefold over the coding sequence of the wild type gene. The increase in expression is not limited to cell cultures of mammalian cells but was also observed in vivo in a mouse model. It is expected that the observed improvement in expression of the codon-optimised DNAI1 coding sequence will result in an improved, more cost-effective mRNA replacement therapy for patients suffering from PCD, because it does not require the use of modified nucleotides for the preparation of the mRNA and allows treatment with a reduced dose and/or at extended dosing intervals.

In some embodiments, codon-optimized mRNA sequences according to the present invention were further codon-optimized by a new process: the process first generates a list of codon-optimized sequences and then applies three filters to the list. Specifically, it applies a motif screen filter, guanine-cytosine (GC) content analysis filter, and codon adaptation index (CAI) analysis filter to produce an updated list of optimized nucleotide sequences. The updated list no longer includes nucleotide sequences containing features that are expected to interfere with effective transcription and/or translation of the encoded protein antigen.

### Exemplary Codon Optimized DNAI1 mRNA Sequences

The sequences that follow recite select, exemplary codon-optimized DNAI1 mRNA sequences.

In some embodiments, a suitable mRNA may be a codon-optimized sequence, as shown in SEQ ID NO: 6-10.

In some embodiments, a suitable mRNA sequence may be an mRNA sequence a homolog or an analog of human DNAI1 protein. For example, a homolog or an analog of human DNAI1 protein may be a modified human DNAI1 protein containing one or more amino acid substitutions, deletions, and/or insertions as compared to a wild-type or naturally-occurring human DNAI1 protein while retaining substantial DNAI1 protein activity. In some embodiments, an mRNA suitable for the present invention encodes an amino acid sequence at least 70%, 75%, 80%, 85%, 90%, 91%, 92%, 93%, 94%, 95%, 96%, 97%, 98%, 99% or more homologous to SEQ ID NO: 1. In some embodiments, an mRNA suitable for the present invention encodes a protein substantially identical to human DNAI1 protein. In some embodiments, an mRNA suitable for the present invention encodes an amino acid sequence at least 70%, 75%, 80%, 85%, 90%, 91%, 92%, 93%, 94%, 95%, 96%, 97%, 98%, 99% or more identical to SEQ ID NO: 1. Typically, an mRNA according to the present invention encodes a DNAI1 protein with an amino acid sequence that is identical to SEQ ID NO: 1.

In some embodiments, an mRNA suitable for the present invention encodes a fragment or a portion of human DNAI1 protein. In some embodiments, an mRNA suitable for the present invention encodes a fragment or a portion of human DNAI1 protein, wherein the fragment or portion of the protein still maintains DNAI1 activity similar to that of the wild-type protein.

In some embodiments, a suitable mRNA encodes a fusion protein comprising a full length, fragment or portion of a DNAI1 protein fused to another protein (e.g., an N or C terminal fusion). In some embodiments, the protein fused to the mRNA encoding a full length, fragment or portion of a DNAI1 protein encodes a signal or a cellular targeting sequence.

In some embodiments, an mRNA suitable for the present invention comprises a nucleotide sequence at least 85%, 90%, 91%, 92%, 93%, 94%, 95%, 96%, 97%, 98%, 99% or more identical to SEQ ID NO: 6, SEQ ID NO: 7, SEQ ID NO: 8, SEQ ID NO: 9, or SEQ ID NO: 10. In some embodiments, an mRNA in accordance with the present invention comprises a nucleotide sequence at least 95 % identical to SEQ ID NO: 6. In some embodiments, an mRNA according to the present invention comprises a nucleotide sequence at least 99 % identical to SEQ ID NO: 6. For example, an mRNA according to the present invention comprises the nucleotide sequence of SEQ ID NO: 6. In some embodiments, an mRNA in accordance with the present invention comprises a nucleotide sequence at least 95 % identical to SEQ ID NO: 7. In some embodiments, an mRNA according to the present invention comprises a nucleotide sequence at least 99 % identical to SEQ ID NO: 7. For example, an mRNA according to the present invention comprises the nucleotide sequence of SEQ ID NO: 7. In some embodiments, an mRNA in accordance with the present invention comprises a nucleotide sequence at least 95 % identical to SEQ ID NO: 8. In some embodiments, an mRNA according to the present invention comprises a nucleotide sequence at least 99 % identical to SEQ ID NO: 8. For example, an mRNA according to the present invention comprises the nucleotide sequence of SEQ ID NO: 8. In some embodiments, an mRNA in accordance with the present invention comprises a nucleotide sequence at least 95 % identical to SEQ ID NO: 9. In some embodiments, an mRNA according to the present invention comprises a nucleotide sequence at least 99 % identical to SEQ ID NO: 9. For example, an mRNA according to the present invention comprises the nucleotide sequence of SEQ ID NO: 9. In some embodiments, an mRNA in accordance with the present invention comprises a nucleotide sequence at least 95 % identical to SEQ ID NO: 10. In some embodiments, an mRNA according to the present invention comprises a nucleotide sequence at least 99 % identical to SEQ ID NO: 10. For example, an mRNA according to the present invention comprises the nucleotide sequence of SEQ ID NO: 10.

Exemplary codon-optimized DNAI1 mRNA sequences are shown in Table 3:

TABLE 3

Exemplary DNAI1 Codon-Optimized Sequence

| | |
|---|---|
| SEQ ID NO: 6 | ATGATCCCCGCCTCCGCCAAAGCCCCTCACAAGCAACCGCACAAGCAAA GCATTAGCATTGGGCGGGGTACTCGGAAGCGCGACGAGGACTCGGGAA CTGAAGTCGGAGAGGGGACCGACGAATGGGCGCAGTCAAAGGCCACCG TGCGCCCACCGGACCAGCTCGAGCTGACCGATGCTGAGCTGAAGGAGGA GTTTACCCGGATCCTGACAGCCAACAACCCACATGCACCGCAGAACATC GTGCGGTACAGCTTCAAAGAGGGAACTTATAAGCCCATTGGCTTCGTGA ACCAACTCGCGGTGCATTACACCCAAGTCGGAAACCTTATTCCGAAGGA CTCGGACGAAGGCAGACGCCAGCACTACCGGGACGAGCTCGTGGCAGG ATCCCAGGAAAGCGTCAAGGTCATTTCCGAGACTGGCAACCTCGAGGAG GACGAAGAACCTAAGGAGCTGGAAACCGAACCCGGATCCCAGACCGAC GTGCCGGCCGCTGGGGCTGCCGAGAAAGTCACTGAAGAGGAACTCATGA CCCCGAAGCAGCCGAAAGAGAGAAAGCTCACCAACCAATTCAACTTCAG CGAGCGCGCCAGCCAAACCTACAACAACCCAGTCAGGGATCGGGAATGT CAGACCGAACCGCCTCCGAGAACGAACTTCTCGGCGACCGCGAACCAAT GGGAGATCTACGACGCCTACGTGGAAGAACTGGAAAAGCAGGAAAAGA CTAAGGAAAAGGAAAAGGCCAAGACTCCCGTCGCCAAGAAGTCGGGCA AAATGGCCATGCGGAAGCTCACCTCCATGGAATCACAGACTGACGACTT GATCAAGTTGAGCCAGGCCGCAAAGATCATGGAGCGCATGGTCAACCAA AATACTTACGACGATATCGCCCAAGACTTCAAGTACTACGACGACGCTG CCGATGAATACCGAGATCAAGTCGGCACCCTACTGCCGCTTTGGAAGTT CCAGAATGACAAGGCCAAGAGGCTGAGCGTGACCGCGCTGTGCTGGAA |
| | CCCCAAATACCGCGACCTCTTCGCCGTGGGATACGGCTCCTACGATTTCA TGAAGCAGAGCCGGGGAATGTTGCTCCTTTACTCCCTGAAGAACCCCTC CTTCCCTGAGTACATGTTCAGCTCAAACAGCGGCGTGATGTGCCTCGACA TTCACGTGGACCACCCTTACCTCGTGGCCGTGGGTCACTACGACGGCAA CGTCGCGATCTACAACTTGAAGAAGCCGCATTCACAGCCCTCGTTTTGCT CCTCGGCCAAGTCCGGCAAACATTCGGACCCAGTGTGGCAAGTCAAGTG GCAGAAAGATGACATGGACCAAAACTTGAACTTCTTCAGCGTGTCCTCC GACGGACGGATCGTGTCCTGGACCCTCGTGAAGCGGAAGTTGGTGCATA TCGACGTGATCAAATTGAAGGTCGAGGGTTCGACCACCGAAGTGCCTGA AGGCCTGCAGCTTCACCCCGTGGGATGCGGCACTGCCTTCGACTTCCACA AGGAGATCGACTACATGTTCCTCGTGGGAACCGAGGAAGGGAAGATCTA CAAATGCAGCAAGTCCTACTCATCACAATTCCTGGATACCTACGATGCCC ACAACATGAGCGTGGATACCGTGTCGTGGAACCCCTATCACACCAAGGT ATTCATGTCCTGCTCCTCCGACTGGACCGTCAAGATTTGGGACCACACCA TCAAGACCCCCATGTTCATCTACGACCTGAACTCCGCCGTGGGGGATGT GGCCTGGGCCCCCTACTCGTCGACCGTGTTTGCCGCGGTCACCACGGAC GGAAAGGCACACATTTTCGACCTTGCGATTAACAAATACGAGGCGATTT GCAACCAGCCCGTGGCCGCCAAAAAGAACCGCCTGACCCACGTTCAATT CAACTTAATCCACCCAATCATCATCGTCGGCGATGACAGAGGACACATT ATTAGCCTGAAACTTAGCCCCAACCTCCGCAAGATGCCCAAGGAGAAGA AGGGACAGGAAGTCCAGAAGGGCCCTGCCGTGGAGATTGCAAAGCTCG ATAAGCTCCTGAACTTAGTCCGGGAAGTGAAGATCAAGACTTAA |
| SEQ ID NO: 7 | ATGATCCCCGCTAGCGCTAAGGCCCCTCACAAACAGCCCCACAAGCAGA GCATTAGCATTGGACGCGGCACACGGAAACGGGACGAAGATTCCGGAA CCGAAGTCGGGGAAGGCACCGATGAATGGGCCCAGTCCAAGGCCACCG TGCGGCCTCCTGACCAATTGGAACTGACTGACGCGGAACTGAAGGAAGA ATTCACCAGAATTCTAACCGCCAACAATCCTCATGCCCCGCAAAACATC GTGCGCTACTCATTCAAGGAAGGAACCTACAAGCCCATTGGATTCGTGA ACCAACTCGCCGTGCACTACACTCAAGTCGGCAACTTAATTCCCAAGGA TTCGGACGAGGGCCGCCGGCAGCACTACAGGGATGAGCTTGTGGCAGGC TCACAGGAAAGCGTCAAAGTCATTAGCGAAACCGGCAACCTTGAAGAG GATGAGGAGCCTAAGGAGCTGGAAACCGAGCCGGGTTCGCAAACCGAC GTGCCTGCCGCCGGAGCCGCAGAGAAGGTCACCGAAGAGGAACTCATG ACCCCCAAGCAGCCGAAGGAGCGCAAACTCACTAACCAATTCAACTTCA GCGAGAGAGCGAGCCAGACCTACAACAACCCAGTGCGCGACCGGGAAT GCCAGACTGAGCCGCCCCCGAGAACTAACTTCAGCGCGACTGCCAACCA ATGGGAGATCTACGACGCCTACGTGGAAGAGTTGGAGAAGCAGGAAAA GACCAAGGAGAAGGAAAAGGCCAAGACCCCGGTGGCGAAGAAGTCCGG AAAGATGGCGATGCGGAAGTTGACGTCCATGGAATCCCAGACCGATGAC CTCATCAAGCTCAGCCAGGCAGCAAAGATCATGGAAAGGATGGTGAACC AGAACACCTACGATGACATTGCTCAGGACTTTAAGTACTACGACGATGC |
| | CGCCGACGAATACCGCGACCAGGTCGGAACCCTCCTCCCGCTTTGGAAA TTCCAGAACGATAAAGCCAAGCGCCTCAGCGTGACTGCGCTGTGCTGGA ACCCGAAGTACCGGGACCTTTTCGCCGTGGGCTATGGATCCTACGACTTC ATGAAGCAATCGCGGGGGATGCTCCTCCTGTATAGCCTGAAGAACCCGA GCTTCCCCGAGTACATGTTCAGCTCAAACTCCGGAGTGATGTGCCTCGAC ATCCACGTGGACCACCCATACTTGGTGGCCGTGGGCCACTACGATGGCA ATGTCGCGATCTACAACCTCAAGAAGCCCCACTCCCAACCCTCGTTCTGC AGCAGCGCCAAGAGCGGGAAACACTCCGACCCAGTGTGGCAAGTCAAA TGGCAGAAGGACGACATGGATCAGAACTTGAACTTCTTCAGCGTGTCCT CAGACGGACGGATCGTGTCCTGGACCCTCGTGAAGAGGAAGTTGGTCCA TATCGACGTGATCAAGCTCAAGGTCGAGGGTTCAACCACCGAGGTGCCC GAGGGCCTGCAGCTCCACCCCGTGGGCTGCGGCACGGCCTTCGACTTTC ATAAGGAAATTGACTACATGTTCCTCGTGGGGACTGAAGAGGGAAAGAT CTACAAGTGCAGCAAATCCTACTCGTCCCAATTCCTCGACACCTACGATG CGCACAACATGAGCGTCGACACGGTGTCATGGAACCCCTACCATACAAA GGTATTCATGTCGTGCTCCTCCGACTGGACCGTGAAGATTTGGGATCACA CTATCAAGACCCCGATGTTCATATACGACCTGAACTCCGCCGTGGGAGA TGTGGCTTGGGCGCCCTACTCATCGACCGTGTTTGCCGCCGTGACCACCG ACGGAAAGGCTCACATTTTCGATCTTGCCATCAACAAGTACGAGGCGAT TTGCAACCAGCCAGTGGCCGCCAAGAAGAACAGACTGACTCACGTGCAG TTTAACTTAATCCACCCCATTATTATCGTGGGGGACGACCGCGGACACAT TATTAGCCTGAAGCTCAGCCCGAACCTCCGGAAGATGCCTAAGGAGAAG AAGGGGCAGGAAGTGCAGAAAGGCCCGGCCGTGGAAATTGCCAAATTG GACAAGTTACTGAACCTTGTCCGCGAAGTCAAGATCAAAACTTAA |
| SEQ ID NO: 8 | ATGATCCCCGCATCCGCCAAAGCCCCTCATAAACAGCCCCACAAACAGT CCATCTCCATTGGACGGGGGACCCGGAAAAGGGATGAGGACTCTGGGAC GGAAGTTGGAGAAGGCACTGACGAATGGGCACAGAGTAAGGCTACCGT GAGACCTCCCGACCAGCTGGAGCTCACTGACGCAGAACTGAAGGAGGA GTTTACTAGGATCCTGACAGCAAATAACCCCCACGCCCCACAGAATATC GTCAGATATAGCTTCAAAGAGGGCACATACAAGCCTATTGGGTTCGTGA ACCAGCTGGCTGTGCATTACACACAGGTGGGGAACCTTATTCCTAAAGA CTCTGATGAAGGCCGCAGACAGCATTATAGAGATGAACTGGTTGCAGGA TCCCAAGAGTCTGTGAAAGTGATTAGCGAGACCGGCAACCTGGAAGAAG ATGAGGAACCAAAAGAACTGGAGACAGAGCCTGGGTCTCAGACAGACG TGCCAGCAGCTGGCGCTGCCGAGAAAGTGACAGAGGAGGAGCTGATGA CACCTAAACAGCCAAAAGAGAGGAAGCTGACAAACCAATTCAATTTTTC CGAACGGGCATCACAGACCTACAACAACCCAGTGCGCGACCGGGAGTGT CAAACCGAACCTCCTCCTAGAACAAACTTTTCTGCTACTGCAAATCAGTG GGAGATCTACGATGCCTACGTGGAGGAGCTGGAGAAGCAGGAAAAGAC TAAGGAGAAGGAGAAGGCAAAGACCCCCGTGGCCAAAAAATCCGGCAA AATGGCAATGCGGAAGCTGACTTCTATGGAAAGCCAGACTGATGACCTG |
| | ATCAAACTGTCCCAGGCAGCTAAGATTATGGAAAGGATGGTCAATCAGA ATACATATGACGACATTGCTCAGGACTTTAAGTATTATGATGATGCCGCT GACGAGTATCGGGACCAAGTGGGGACACTGCTGCCACTGTGGAAGTTTC AAAACGACAAGGCTAAAAGGCTGTCCGTGACAGCACTCTGCTGGAATCC CAAGTACCGGGACCTCTTTGCCGTGGGGTACGGATCTTACGACTTCATGA AACAGTCCAGAGGCATGCTGCTGCTGTACAGCTTGAAGAACCCCTCCTTT CCCGAGTACATGTTCAGCTCTAATTCTGGAGTGATGTGCCTGGACATCCA CGTGGATCACCCTTACCTCGTGGCCGTTGGACACTATGACGGCAATGTG GCCATCTACAACCTGAAAAAACCACACTCTCAGCCTTCCTTTTGTAGCTC TGCAAAGTCCGGAAAGCATTCCGACCCCGTGTGGCAAGTGAAATGGCAG AAAGACGACATGGACCAGAATCTGAACTTCTTCTCCGTCTCTTCAGACG GCAGAATCGTCTCATGGACTCTGGTCAAACGGAAGCTGGTTCACATCGA CGTGATCAAACTCAAGGTCGAAGGATCGACTACTGAGGTGCCAGAAGGA CTGCAGCTGCACCCAGTGGGATGTGGAACTGCATTTGATTTCCATAAAG AAATCGACTACATGTTTCTGGTGGGAACTGAAGAGGGGAAGATCTATAA GTGTAGCAAATCCTATTCTAGCCAGTTTCTGGATACATACGACGCTCACA ACATGTCCGTGGACACTGTAAGCTGGAACCCCTATCATACCAAGGTGTT CATGTCCTGCAGCTCCGATTGGACTGTTAAGATTTGGGATCACACAATCA AGACCCCTATGTTTATCTACGATCTGAACTCTGCCGTGGGGGATGTGGCC TGGGCACCATATAGCTCCACAGTCTTCGCAGCTGTCACTACCGATGGAA AGGCCCACATTTTTGACCTGGCTATCAACAAATACGAGGCCATCTGCAA TCAGCCTGTGGCAGCAAAGAAGAACCGCCTGACTCACGTGCAATTCAAC CTGATTCACCCTATCATCATTGTTGGGGATGATAGGGGCCACATTATTTC TCTAAAGCTGTCCCCAAATCTGCGGAAAATGCCCAAGGAGAAGAAAGGC CAGGAGGTGCAGAAAGGCCCAGCCGTTGAAATCGCAAAGCTGGACAAG CTGCTCAACCTCGTCCGGGAGGTTAAAATCAAAACCTGA |
| SEQ ID NO: 9 | ATGATCCCAGCAAGCGCCAAGGCCCCACACAAACAGCCCCACAAGCAGT CGATCAGCATTGGCAGGGGGACTCGCAAGAGAGACGAGGACTCCGGAA CAGAAGTGGGGGAGGGGACAGATGAATGGGCCCAGTCTAAGGCCACTG TTCGCCCTCCGGATCAGCTGGAACTGACAGATGCCGAGCTGAAGGAAGA GTTCACCAGGATTCTGACTGCAAATAATCCACACGCTCCACAGAACATT GTGAGATATTCTTTTAAGGAGGGCACTTACAAACCCATCGGGTTTGTGA ATCAGCTGGCAGTGCATTACACTCAAGTGGGCAACCTGATCCCCAAAGA CTCTGATGAAGGGAGGCGGCAGCACTATAGGGACGAGCTGGTCGCTGGG TCCCAAGAGAGCGTGAAAGTCATTTCTGAGACTGGCAACCTGGAAGAGG ATGAGGAGCCAAAGGAGCTGGAGACTGAACCAGGGTCTCAGACAGATG TGCCCGCCGCTGGAGCTGCTGAGAAGGTGACAGAGGAGGAACTGATGA CCCCTAAACAGCCTAAGGAACGGAAGCTCACCAACCAGTTCAACTTCAG CGAAAGAGCTAGCCAGACTTATAATAACCCTGTGCGCGACCGGGAGTGT CAGACTGAGCCCCCACCAAGAACCAATTTCTCCGCCACTGCCAACCAGT GGGAAATCTATGACGCTTACGTCGAGGAGCTGGAGAAACAGGAGAAAA |
| | CTAAGGAGAAAGAAAAGGCCAAAACACCCGTCGCCAAAAAGTCTGGCA AGATGGCCATGAGAAAACTGACCTCCATGGAGTCTCAGACCGACGACCT GATCAAACTGTCCCAGGCAGCCAAGATCATGGAGAGGATGGTGAACCA GAACACCTATGATGACATTGCCCAGGACTTTAAATACTACGATGATGCC GCTGACGAGTATCGGGACCAGGTGGGGACTCTGCTGCCTCTGTGGAAAT TCCAGAATGATAAGGCTAAACGCCTGTCCGTGACCGCCCTCTGCTGGAA CCCTAAGTACCGCGACCTCTTTGCTGTGGGGTACGGATCTTACGACTTCA TGAAACAGTCCAGAGGCATGCTGCTGCTGTACAGCTTGAAGAACCCCTC CTTTCCCGAGTACATGTTCAGCTCTAATTCTGGAGTGATGTGCCTGGACA TCCACGTGGATCACCCTTACCTCGTGGCCGTTGGACACTATGACGGCAAT GTGGCCATCTACAACCTGAAAAAACCACACTCTCAGCCTTCCTTTTGTAG CTCTGCAAAGTCCGGAAAGCATTCCGACCCCGTGTGGCAAGTGAAATGG CAGAAAGACGACATGGACCAGAATCTGAACTTCTTCTCCGTCTCTTCAG ACGGCAGAATCGTCTCATGGACTCTGGTCAAACGGAAGCTGGTTCACAT CGACGTGATCAAACTCAAGGTCGAAGGATCGACTACTGAGGTGCCAGAA GGACTGCAGCTGCACCCAGTGGGATGTGGAACTGCATTTGATTTCCATA AAGAAATCGACTACATGTTTCTGGTGGGAACTGAAGAGGGGAAGATCTA TAAGTGTAGCAAATCCTATTCTAGCCAGTTTCTGGATACATACGACGCTC ACAACATGTCCGTGGACACTGTAAGCTGGAACCCCTATCATACCAAGGT GTTCATGTCCTGCAGCTCCGATTGGACTGTTAAGATTTGGGATCACACAA TCAAGACCCCTATGTTTATCTACGATCTGAACTCTGCCGTGGGGGATGTG GCCTGGGCACCATATAGCTCCACAGTCTTCGCAGCTGTCACTACCGATGG AAAGGCCCACATTTTTGACCTGGCTATCAACAAATACGAGGCCATCTGC AATCAGCCTGTGGCAGCAAAGAAGAACCGCCTGACTCACGTGCAATTCA ACCTGATTCACCCTATCATCATTGTTGGGGATGATAGGGGCCACATTATT TCTCTAAAGCTGTCCCCAAATCTGCGGAAAATGCCCAAGGAGAAGAAAG GCCAGGAGGTGCAGAAAGGCCCAGCCGTTGAAATCGCAAAGCTGGACA AGCTGCTCAACCTCGTCCGGGAGGTTAAAATCAAAACCTGA |
| SEQ ID NO: 10 | ATGATCCCAGCTTCTGCCAAGGCCCCACACAAGCAGCCACACAAACAGA GCATTTCCATTGGGCGCGGCACAAGGAAGAGAGACGAGGACTCAGGCA CAGAGGTGGGCGAAGGAACCGACGAGTGGGCTCAGAGCAAAGCCACAG TGAGGCCCCCAGATCAGCTGGAGCTGACAGACGCCGAGCTGAAGGAGG AGTTTACCCGCATCCTGACTGCCAATAACCCACACGCACCCCAGAACAT CGTGCGCTATTCTTTTAAGGAAGGAACCTATAAGCCAATCGGCTTTGTCA ATCAGCTGGCTGTGCACTACACCCAGGTTGGGAACCTGATCCCCAAGGA TAGCGACGAGGGCAGGAGACAGCATTATAGAGACGAGCTCGTCGCCGG AAGCCAGGAGTCTGTCAAAGTGATCAGCGAAACAGGAAACCTGGAGGA GGATGAGGAGCCCAAGGAACTGGAAACCGAGCCTGGCAGCCAGACAGA TGTGCCAGCCGCAGGAGCCGCAGAGAAGGTGACAGAAGAGGAGCTCAT GACCCCCAAACAGCCAAAGGAGCGGAAACTGACAAACCAGTTCAACTTC AGCGAAAGAGCCAGCCAGACCTACAATAACCCCGTGCGGGACAGAGAA |
| | TGCCAGACAGAGCCTCCACCACGCACCAACTTCTCCGCAACAGCTAACC AGTGGGAGATCTATGATGCCTACGTGGAGGAGCTGGAAAAGCAGGAGA AGACCAAAGAAAAGGAGAAAGCCAAGACCCCTGTCGCCAAGAAGTCCG GCAAAATGGCTATGAGAAAGCTGACATCTATGGAATCCCAGACTGATGA CCTGATCAAGCTGTCTCAGGCAGCCAAGATTATGGAAAGAATGGTGAAT CAGAACACCTATGACGACATCGCCCAGGATTTTAAGTACTATGATGACG CTGCAGACGAGTATAGAGATCAGGTGGGGACCCTGCTGCCACTGTGGAA GTTCCAGAATGACAAGGCTAAGCGCCTGTCCGTGACAGCTCTGTGCTGG AATCCAAAATATAGGGACCTCTTCGCCGTGGGCTACGGCTCTTATGACTT CATGAAGCAGTCACGCGGGATGCTGCTGCTGTACAGCCTGAAAAATCCC TCCTTTCCCGAGTACATGTTCAGCTCTAACTCCGGGGTCATGTGTCTGGA TATTCATGTGGACCATCCATACCTGGTGGCTGTCGGGCACTACGATGGA AACGTGGCTATCTACAATCTGAAGAAGCCACACTCCCAGCCCTCCTTTTG CTCCTCCGCCAAGTCCGGCAAGCACTCCGACCCTGTGTGGCAGGTCAAG TGGCAGAAGGACGACATGGACCAGAACCTGAACTTCTTTTCTGTGTCTA GCGATGGCAGGATCGTGTCCTGGACCCTGGTGAAGAGAAAACTGGTGCA CATCGATGTTATCAAGCTCAAAGTCGAGGGAAGCACCACCGAGGTTCCT GAGGGCCTGCAGCTGCACCCAGTGGGCTGCGGCACAGCCTTCGACTTTC ATAAAGAGATTGACTACATGTTCCTGGTGGGCACAGAGGAGGGGAAGAT CTACAAGTGCTCCAAATCCTACTCCAGCCAGTTTCTGGACACTTACGACG CTCATAATATGAGCGTGGACACCGTGTCCTGGAACCCTTACCACACAAA GGTGTTCATGAGCTGCAGCAGCGACTGGACTGTGAAGATTTGGGACCAT ACTATCAAAACCCCAATGTTTATCTATGATCTCAATTCTGCCGTGGGCGA CGTGGCTTGGGCCCCCTATTCCTCCACAGTGTTCGCAGCCGTGACTACCG ACGGAAAAGCCCACATTTTCGACCTCGCTATTAACAAGTATGAGGCCAT TTGTAACCAGCCAGTGGCTGCCAAGAAGAACCGCCTGACCCACGTGCAG TTCAACCTGATTCACCCAATTATCATTGTGGGGGACGACAGAGGACACA TTATCTCACTGAAGCTGTCTCCTAATCTGAGAAAGATGCCTAAGGAGAA GAAAGGACAGGAGGTGCAGAAGGGCCCTGCCGTGGAAATTGCCAAACT CGACAAGCTGCTGAACCTGGTGAGGGAGGTGAAGATCAAGACATGA |

### mRNA Synthesis

mRNAs according to the present invention may be synthesized according to any of a variety of known methods. Various methods are described in published U.S. Application No. US 2018/0258423, and can be used to practice the present invention, all of which are incorporated herein by reference. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

### 5′ And 3′ Untranslated Region

In some embodiments, mRNAs include a 5′ untranslated region (UTR). In some embodiments, mRNAs include a 3′ untranslated region. In some embodiments, mRNAs include both a 5′ untranslated region and a 3′ untranslated region. In some embodiments, a 5′ untranslated region includes one or more elements that affect an mRNA’s stability or translation, for example, an iron responsive element. In some embodiments, a 5′ untranslated region may be between about 50 and 500 nucleotides in length.

In some embodiments, a 3′ untranslated region includes one or more of a polyadenylation signal, a binding site for proteins that affect an mRNA’s stability of location in a cell, or one or more binding sites for miRNAs. In some embodiments, a 3′ untranslated region may be between 50 and 500 nucleotides in length or longer.

Exemplary 3′ and 5′ untranslated region sequences can be derived from mRNA molecules which are stable (e.g., globin, actin, GAPDH, tubulin, histone, or citric acid cycle enzymes) to increase the stability of the sense mRNA molecule. For example, a 5′ UTR sequence may include a partial sequence of a CMV immediate-early 1 (IE1) gene, or a fragment thereof to improve the nuclease resistance and/or improve the half-life of the polynucleotide. Also contemplated is the inclusion of a sequence encoding human growth hormone (hGH), or a fragment thereof to the 3′ end or untranslated region of the polynucleotide (e.g., mRNA) to further stabilize the polynucleotide. Generally, these modifications improve the stability and/or pharmacokinetic properties (e.g., half-life) of the polynucleotide relative to their unmodified counterparts, and include, for example modifications made to improve such polynucleotides’ resistance to in vivo nuclease digestion.

In certain embodiments, the codon-optimized DNAI1 mRNA includes a coding region having a codon-optimized coding region flanked by 5′ and 3′ untranslated regions as represented as X and Y, respectively (vide infra)

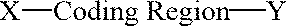

where the coding region sequence is SEQ ID NO: 6, or a sequence 70%, 75%, 80%, 85%, 90%, 91%, 92%, 93%, 94%, 95%, 96%, 97%, 98%, 99% or more identical to SEQ ID NO: 6; SEQ ID NO: 7 SEQ ID NO: 8; SEQ ID NO: 9; or SEQ ID NO: 10 or a sequence 70%, 75%, 80%, 85%, 90%, 91%, 92%, 93%, 94%, 95%, 96%, 97%, 98%, 99% or more identical to SEQ ID NO: 10; and where
X (5′ UTR Sequence) is
AGACAGAUCGCCUGGAGACGCCAUCCACGCUGUUUUGACCUCCAUAGAAGACA CCGGGACCGAUCCAGCCUCCGCGGCCGGGAACGGUGCAUUGGAACGCGGAUUC CCCGUGCCAAGAGUGACUCACCGUCCUUGACACG [SEQ ID NO.: 2] or a sequence 70%, 75%, 80%, 85%, 90%, 91%, 92%, 93%, 94%, 95%, 96%, 97%, 98%, 99% or more identical to SEQ ID NO: 2, or
GGACAGAUCGCCUGGAGACGCCAUCCACGCUGUUUUGACCUCCAUAGAAGACA CCGGGACCGAUCCAGCCUCCGCGGCCGGGAACGGUGCAUUGGAACGCGGAUUC CCCGUGCCAAGAGUGACUCACCGUCCUUGACACG (SEQ ID NO: 3) or a sequence 70%, 75%, 80%, 85%, 90%, 91%, 92%, 93%, 94%, 95%, 96%, 97%, 98%, 99% or more identical to SEQ ID NO: 3; and where Y (3′ UTR Sequence) is
CGGGUGGCAUCCCUGUGACCCCUCCCCAGUGCCUCUCCUGGCCCUGGAAGUUG CCACUCCAGUGCCCACCAGCCUUGUCCUAAUAAAAUUAAGUUGCAUCAAGCU (SEQ ID NO: 4) or a sequence 70%, 75%, 80%, 85%, 90%, 91%, 92%, 93%, 94%, 95%, 96%, 97%, 98%, 99% or more identical to SEQ ID NO: 4, or
GGGUGGCAUCCCUGUGACCCCUCCCCAGUGCCUCUCCUGGCCCUGGAAGUUGC CACUCCAGUGCCCACCAGCCUUGUCCUAAUAAAAUUAAGUUGCAUCAAAGCU (SEQ ID NO: 5) or a sequence 70%, 75%, 80%, 85%, 90%, 91%, 92%, 93%, 94%, 95%, 96%, 97%, 98%, 99% or more identical to SEQ ID NO: 5.

The present invention may be used to deliver mRNAs of a variety of lengths. In some embodiments, the present invention may be used to deliver in vitro synthesized mRNA of or greater than about 0.5 kb, 1 kb, 1.5 kb, 2 kb, 2.5 kb, 3 kb, 3.5 kb, 4 kb, 4.5 kb, 5 kb 6 kb, 7 kb, 8 kb, 9 kb, 10 kb, 11 kb, 12 kb, 13 kb, 14 kb, 15 kb, 20 kb, 30 kb, 40 kb, or 50 kb in length. In some embodiments, the present invention may be used to deliver in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-50 kb in length.

In some embodiments, for the preparation of mRNA according to the invention, a DNA template is transcribed in vitro. A suitable DNA template typically has a promoter, for example a T3, T7 or SP6 promoter, for in vitro transcription, followed by desired nucleotide sequence for desired mRNA and a termination signal.

### Nucleotides

Various naturally-occurring or modified nucleosides may be used to produce mRNA according to the present invention. In some embodiments, an mRNA is or comprises naturally-occurring nucleosides (or unmodified nucleotides; e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, a suitable mRNA may contain backbone modifications, sugar modifications and/or base modifications. For example, modified nucleotides may include, but not be limited to, modified purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, .beta.-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g., from the U.S. Pat. No. 4,373,071, U.S. Pat. No. 4,401,796, U.S. Pat. No. 4,415,732, U.S. Pat. No. 4,458,066, U.S. Pat. No. 4,500,707, U.S. Pat. No. 4,668,777, U.S. Pat. No. 4,973,679, U.S. Pat. No. 5,047,524, U.S. Pat. No. 5,132,418, U.S. Pat. No. 5,153,319, U.S. Pat. Nos. 5,262,530 and 5,700,642, the disclosures of which are incorporated by reference in their entirety.

In some embodiments, the mRNA comprises one or more nonstandard nucleotide residues. The nonstandard nucleotide residues may include, e.g., 5-methylcytidine (“5mC”), pseudouridine (“DU”), and/or 2-thio-uridine (“2sU”). See, e.g., U.S. Pat. No. 8,278,036 or WO 2011/012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed U.S. Pat. Publication US 2012/0195936 and international publication WO 2011/012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

In some embodiments, mRNAs may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g., cytidine 5′-O-(1-thiophosphate)), boranophosphates, positively charged guanidinium groups etc., which means by replacing the phosphodiester linkage by other anionic, cationic or neutral groups.

In some embodiments, mRNAs may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

### Post-Synthesis Processing

Typically, a 5′ cap and/or a 3′ tail may be added after the synthesis. The presence of the cap is important in providing resistance to nucleases found in most eukaryotic cells. The presence of a “tail” serves to protect the mRNA from exonuclease degradation.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Additional cap structures are described in published U.S. Application No. US 2016/0032356 and published U.S. Application No. US 2018/0125989, which are incorporated herein by reference.

Typically, a tail structure includes a poly(A) and/or poly(C) tail. A poly-A or poly-C tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly A or poly C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly (A) and poly (C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

As described herein, the addition of the 5′ cap and/or the 3′ tail facilitates the detection of abortive transcripts generated during in vitro synthesis because without capping and/or tailing, the size of those prematurely aborted mRNA transcripts can be too small to be detected. Thus, in some embodiments, the 5′ cap and/or the 3′ tail are added to the synthesized mRNA before the mRNA is tested for purity (e.g., the level of abortive transcripts present in the mRNA). In some embodiments, the 5′ cap and/or the 3′ tail are added to the synthesized mRNA before the mRNA is purified as described herein. In other embodiments, the 5′ cap and/or the 3′ tail are added to the synthesized mRNA after the mRNA is purified as described herein.

mRNA synthesized according to the present invention may be used without further purification. In particular, mRNA synthesized according to the present invention may be used without a step of removing shortmers. In some embodiments, mRNA synthesized according to the present invention may be further purified. Various methods may be used to purify mRNA synthesized according to the present invention. For example, purification of mRNA can be performed using centrifugation, filtration and /or chromatographic methods. In some embodiments, the synthesized mRNA is purified by ethanol precipitation or filtration or chromatography, or gel purification or any other suitable means. In some embodiments, the mRNA is purified by HPLC. In some embodiments, the mRNA is extracted in a standard phenol: chloroform : isoamyl alcohol solution, well known to one of skill in the art. In some embodiments, the mRNA is purified using Tangential Flow Filtration. Suitable purification methods include those described in published U.S. Application No. US 2016/0040154, published U.S. Application No. US 2015/0376220, published U.S. Application No. US 2018/0251755, published U.S. Application No. US 2018/0251754, U.S. Provisional Application No. 62/757,612 filed on Nov. 8, 2018, and U.S. Provisional Application No. 62/891,781 filed on Aug. 26, 2019, all of which are incorporated by reference herein and may be used to practice the present invention.

In some embodiments, the mRNA is purified before capping and tailing. In some embodiments, the mRNA is purified after capping and tailing. In some embodiments, the mRNA is purified both before and after capping and tailing.

In some embodiments, the mRNA is purified either before or after or both before and after capping and tailing, by centrifugation.

In some embodiments, the mRNA is purified either before or after or both before and after capping and tailing, by filtration.

In some embodiments, the mRNA is purified either before or after or both before and after capping and tailing, by Tangential Flow Filtration (TFF).

In some embodiments, the mRNA is purified either before or after or both before and after capping and tailing by chromatography.

### Characterization of Purified mRNA

The mRNA composition described herein is substantially free of contaminants comprising short abortive RNA species, long abortive RNA species, double-stranded RNA (dsRNA), residual plasmid DNA, residual in vitro transcription enzymes, residual solvent and/or residual salt.

The mRNA composition described herein has a purity of about between 60% and about 100%. Accordingly, in some embodiments, the purified mRNA has a purity of about 60%. In some embodiments, the purified mRNA has a purity of about 65%. In some embodiments, the purified mRNA has a purity of about 70%. In some embodiments, the purified mRNA has a purity of about 75%. In some embodiments, the purified mRNA has a purity of about 80%. In some embodiments, the purified mRNA has a purity of about 85%. In some embodiments, the purified mRNA has a purity of about 90%. In some embodiments, the purified mRNA has a purity of about 91%. In some embodiments, the purified mRNA has a purity of about 92%. In some embodiments, the purified mRNA has a purity of about 93%. In some embodiments, the purified mRNA has a purity of about 94%. In some embodiments, the purified mRNA has a purity of about 95%. In some embodiments, the purified mRNA has a purity of about 96%. In some embodiments, the purified mRNA has a purity of about 97%. In some embodiments, the purified mRNA has a purity of about 98%. In some embodiments, the purified mRNA has a purity of about 99%. In some embodiments, the purified mRNA has a purity of about 100%.

In some embodiments, the mRNA composition described herein has less than 10%, less than 9%, less than 8%, less than 7%, less than 6%, less than 5%, less than 4%, less than 3%, less than 2%, less than 1%, less than 0.5%, and/or less than 0.1% impurities other than full-length mRNA. The impurities include IVT contaminants, e.g., proteins, enzymes, DNA templates, free nucleotides, residual solvent, residual salt, double-stranded RNA (dsRNA), prematurely aborted RNA sequences (“shortmers” or “short abortive RNA species”), and/or long abortive RNA species. In some embodiments, the purified mRNA is substantially free of process enzymes.

In some embodiments, the residual plasmid DNA in the purified mRNA of the present invention is less than about 1 pg/mg, less than about 2 pg/mg, less than about 3 pg/mg, less than about 4 pg/mg, less than about 5 pg/mg, less than about 6 pg/mg, less than about 7 pg/mg, less than about 8 pg/mg, less than about 9 pg/mg, less than about 10 pg/mg, less than about 11 pg/mg, or less than about 12 pg/mg. Accordingly, the residual plasmid DNA in the purified mRNA is less than about 1 pg/mg. In some embodiments, the residual plasmid DNA in the purified mRNA is less than about 2 pg/mg. In some embodiments, the residual plasmid DNA in the purified mRNA is less than about 3 pg/mg. In some embodiments, the residual plasmid DNA in the purified mRNA is less than about 4 pg/mg. In some embodiments, the residual plasmid DNA in the purified mRNA is less than about 5 pg/mg. In some embodiments, the residual plasmid DNA in the purified mRNA is less than about 6 pg/mg. In some embodiments, the residual plasmid DNA in the purified mRNA is less than about 7 pg/mg. In some embodiments, the residual plasmid DNA in the purified mRNA is less than about 8 pg/mg. In some embodiments, the residual plasmid DNA in the purified mRNA is less than about 9 pg/mg. In some embodiments, the residual plasmid DNA in the purified mRNA is less than about 10 pg/mg. In some embodiments, the residual plasmid DNA in the purified mRNA is less than about 11 pg/mg. In some embodiments, the residual plasmid DNA in the purified mRNA is less than about 12 pg/mg.

In some embodiments, a method according to the invention removes more than about 90%, 95%, 96%, 97%, 98%, 99% or substantially all prematurely aborted RNA sequences (also known as “shortmers”). In some embodiments, mRNA composition is substantially free of prematurely aborted RNA sequences. In some embodiments, mRNA composition contains less than about 5% (e.g., less than about 4%, 3%, 2%, or 1%) of prematurely aborted RNA sequences. In some embodiments, mRNA composition contains less than about 1% (e.g., less than about 0.9%, 0.8%, 0.7%, 0.6%, 0.5%, 0.4%, 0.3%, 0.2%, or 0.1%) of prematurely aborted RNA sequences. In some embodiments, mRNA composition undetectable prematurely aborted RNA sequences as determined by, e.g., high-performance liquid chromatography (HPLC) (e.g., shoulders or separate peaks), ethidium bromide, Coomassie staining, capillary electrophoresis or Glyoxal gel electrophoresis (e.g., presence of separate lower band). As used herein, the term “shortmers”, “short abortive RNA species”, “prematurely aborted RNA sequences” or “long abortive RNA species” refers to any transcripts that are less than full-length. In some embodiments, “shortmers”, “short abortive RNA species”, or “prematurely aborted RNA sequences” are less than 100 nucleotides in length, less than 90, less than 80, less than 70, less than 60, less than 50, less than 40, less than 30, less than 20, or less than 10 nucleotides in length. In some embodiments, shortmers are detected or quantified after adding a 5′-cap, and/or a 3′-poly A tail. In some embodiments, prematurely aborted RNA transcripts comprise less than 15 bases (e.g., less than 14, 13, 12, 11, 10, 9, 8, 7, 6, 5, 4, or 3 bases). In some embodiments, the prematurely aborted RNA transcripts contain about 8-15, 8-14, 8-13, 8-12, 8-11, or 8-10 bases.

In some embodiments, a purified mRNA of the present invention is substantially free of enzyme reagents used in in vitro synthesis including, but not limited to, T7 RNA polymerase, DNAse I, pyrophosphatase, and/or RNAse inhibitor. In some embodiments, a purified mRNA according to the present invention contains less than about 5% (e.g., less than about 4%, 3%, 2%, or 1%) of enzyme reagents used in in vitro synthesis including. In some embodiments, a purified mRNA contains less than about 1% (e.g., less than about 0.9%, 0.8%, 0.7%, 0.6%, 0.5%, 0.4%, 0.3%, 0.2%, or 0.1%) of enzyme reagents used in in vitro synthesis including. In some embodiments, a purified mRNA contains undetectable enzyme reagents used in in vitro synthesis including as determined by, e.g., silver stain, gel electrophoresis, high-performance liquid chromatography (HPLC), ultra-performance liquid chromatography (UPLC), and/or capillary electrophoresis, ethidium bromide and/or Coomassie staining.

In various embodiments, a purified mRNA of the present invention maintains high degree of integrity. As used herein, the term “mRNA integrity” generally refers to the quality of mRNA after purification. mRNA integrity may be determined using methods well known in the art, for example, by RNA agarose gel electrophoresis. In some embodiments, mRNA integrity may be determined by banding patterns of RNA agarose gel electrophoresis. In some embodiments, a purified mRNA of the present invention shows little or no banding compared to reference band of RNA agarose gel electrophoresis. In some embodiments, a purified mRNA of the present invention has an integrity greater than about 95% (e.g., greater than about 96%, 97%, 98%, 99% or more). In some embodiments, a purified mRNA of the present invention has an integrity greater than 98%. In some embodiments, a purified mRNA of the present invention has an integrity greater than 99%. In some embodiments, a purified mRNA of the present invention has an integrity of approximately 100%.

In some embodiments, the purified mRNA is assessed for one or more of the following characteristics: appearance, identity, quantity, concentration, presence of impurities, microbiological assessment, pH level and activity. In some embodiments, acceptable appearance includes a clear, colorless solution, essentially free of visible particulates. In some embodiments, the identity of the mRNA is assessed by sequencing methods. In some embodiments, the concentration is assessed by a suitable method, such as UV spectrophotometry. In some embodiments, a suitable concentration is between about 90% and 110% nominal (0.9-1.1 mg/mL).

In some embodiments, assessing the purity of the mRNA includes assessment of mRNA integrity, assessment of residual plasmid DNA, and assessment of residual solvent. In some embodiments, acceptable levels of mRNA integrity are assessed by agarose gel electrophoresis. The gels are analyzed to determine whether the banding pattern and apparent nucleotide length is consistent with an analytical reference standard. Additional methods to assess RNA integrity include, for example, assessment of the purified mRNA using capillary gel electrophoresis (CGE). In some embodiments, acceptable purity of the purified mRNA as determined by CGE is that the purified mRNA composition has no greater than about 55% long abortive/degraded species. In some embodiments, residual plasmid DNA is assessed by methods in the art, for example by the use of qPCR. In some embodiments, less than 10 pg/mg (e.g., less than 10 pg/mg, less than 9 pg/mg, less than 8 pg/mg, less than 7 pg/mg, less than 6 pg/mg, less than 5 pg/mg, less than 4 pg/mg, less than 3 pg/mg, less than 2 pg/mg, or less than 1 pg/mg) is an acceptable level of residual plasmid DNA. In some embodiments, acceptable residual solvent levels are not more than 10,000 ppm, 9,000 ppm, 8,000 ppm, 7,000 ppm, 6,000 ppm, 5,000 ppm, 4,000 ppm, 3,000 ppm, 2,000 ppm, 1,000 ppm. Accordingly, in some embodiments, acceptable residual solvent levels are not more than 10,000 ppm. In some embodiments, acceptable residual solvent levels are not more than 9,000 ppm. In some embodiments, acceptable residual solvent levels are not more than 8,000 ppm. In some embodiments, acceptable residual solvent levels are not more than 7,000 ppm. In some embodiments, acceptable residual solvent levels are not more than 6,000 ppm. In some embodiments, acceptable residual solvent levels are not more than 5,000 ppm. In some embodiments, acceptable residual solvent levels are not more than 4,000 ppm. In some embodiments, acceptable residual solvent levels are not more than 3,000 ppm. In some embodiments, acceptable residual solvent levels are not more than 2,000 ppm. In some embodiments, acceptable residual solvent levels are not more than 1,000 ppm.

In some embodiments, microbiological tests are performed on the purified mRNA, which include, for example, assessment of bacterial endotoxins. In some embodiments, bacterial endotoxins are < 0.5 EU/mL, <0.4 EU/mL, <0.3 EU/mL, <0.2 EU/mL or <0.1 EU/mL. Accordingly, in some embodiments, bacterial endotoxins in the purified mRNA are < 0.5 EU/mL. In some embodiments, bacterial endotoxins in the purified mRNA are < 0.4 EU/mL. In some embodiments, bacterial endotoxins in the purified mRNA are < 0.3 EU/mL. In some embodiments, bacterial endotoxins in the purified mRNA are < 0.2 EU/mL. In some embodiments, bacterial endotoxins in the purified mRNA are < 0.2 EU/mL. In some embodiments, bacterial endotoxins in the purified mRNA are < 0.1 EU/mL. In some embodiments, the purified mRNA has not more than 1 CFU/10 mL, 1 CFU/25 mL, 1CFU/50 mL, 1CFU/75 mL, or not more than 1 CFU/100 mL. Accordingly, in some embodiments, the purified mRNA has not more than 1 CFU/10 mL. In some embodiments, the purified mRNA has not more than 1 CFU/25 mL. In some embodiments, the purified mRNA has not more than 1 CFU/50 mL. In some embodiments, the purified mRNA has not more than 1 CFR/75 mL. In some embodiments, the purified mRNA has 1 CFU/100 mL.

In some embodiments, the pH of the purified mRNA is assessed. In some embodiments, acceptable pH of the purified mRNA is between 5 and 8. Accordingly, in some embodiments, the purified mRNA has a pH of about 5. In some embodiments, the purified mRNA has a pH of about 6. In some embodiments, the purified mRNA has a pH of about 7. In some embodiments, the purified mRNA has a pH of about 7. In some embodiments, the purified mRNA has a pH of about 8.

In some embodiments, the translational fidelity of the purified mRNA is assessed. The translational fidelity can be assessed by various methods and include, for example, transfection and Western blot analysis. Acceptable characteristics of the purified mRNA includes banding pattern on a Western blot that migrates at a similar molecular weight as a reference standard.

In some embodiments, the purified mRNA is assessed for conductance. In some embodiments, acceptable characteristics of the purified mRNA include a conductance of between about 50% and 150% of a reference standard.

The purified mRNA is also assessed for Cap percentage and for PolyA tail length. In some embodiments, an acceptable Cap percentage includes Cap1, % Area: NLT90. In some embodiments, an acceptable PolyA tail length is about 100 -1500 nucleotides (e.g., 100, 150, 200, 250, 300, 350, 400, 450, 500, 550, 600, 650, 700, 750, 800, 850, 900, 950, and 1000, 1100, 1200, 1300, 1400, or 1500 nucleotides).

In some embodiments, the purified mRNA is also assessed for any residual PEG. In some embodiments, the purified mRNA has less than between 10 ng PEG/mg of purified mRNA and 1000 ng PEG/mg of mRNA. Accordingly, in some embodiments, the purified mRNA has less than about 10 ng PEG/mg of purified mRNA. In some embodiments, the purified mRNA has less than about 100 ng PEG/mg of purified mRNA. In some embodiments, the purified mRNA has less than about 250 ng PEG/mg of purified mRNA. In some embodiments, the purified mRNA has less than about 500 ng PEG/mg of purified mRNA. In some embodiments, the purified mRNA has less than about 750 ng PEG/mg of purified mRNA. In some embodiments, the purified mRNA has less than about 1000 ng PEG/mg of purified mRNA.

Various methods of detecting and quantifying mRNA purity are known in the art. For example, such methods include, blotting, capillary electrophoresis, chromatography, fluorescence, gel electrophoresis, HPLC, silver stain, spectroscopy, ultraviolet (UV), or UPLC, or a combination thereof. In some embodiments, mRNA is first denatured by a Glyoxal dye before gel electrophoresis (“Glyoxal gel electrophoresis”). In some embodiments, synthesized mRNA is characterized before capping or tailing. In some embodiments, synthesized mRNA is characterized after capping and tailing.

### Delivery Vehicles

According to the present invention, mRNA or MCNA encoding a protein or a peptide (e.g., a full length, fragment, or portion of a protein or a peptide) as described herein may be delivered as naked RNA (unpackaged) or via delivery vehicles. As used herein, the terms “delivery vehicle,” “transfer vehicle,” “nanoparticle” or grammatical equivalent, are used interchangeably.

Delivery vehicles can be formulated in combination with one or more additional nucleic acids, carriers, targeting ligands or stabilizing reagents, or in pharmacological compositions where it is mixed with suitable excipients. Techniques for formulation and administration of drugs may be found in “Remington’s Pharmaceutical Sciences,” Mack Publishing Co., Easton, Pa., latest edition. A particular delivery vehicle is selected based upon its ability to facilitate the transfection of a nucleic acid to a target cell.

In some embodiments, mRNAs or MCNAs encoding at least one protein or peptide may be delivered via a single delivery vehicle. In some embodiments, mRNAs or MCNAs encoding at least one protein or peptide may be delivered via one or more delivery vehicles each of a different composition. In some embodiments, the one or more mRNAs and/or MCNAs are encapsulated within the same lipid nanoparticles. In some embodiments, the one or more mRNAs are encapsulated within separate lipid nanoparticles.

According to various embodiments, suitable delivery vehicles include, but are not limited to polymer based carriers, such as polyethyleneimine (PEI), lipid nanoparticles and liposomes, nanoliposomes, ceramide-containing nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic polyconjugates), dry powder formulations, plasmids, viruses, calcium phosphate nucleotides, aptamers, peptides and other vectorial tags. Also contemplated is the use of bionanocapsules and other viral capsid proteins assemblies as a suitable transfer vehicle. (Hum. Gene Ther. 2008 September; 19(9):887-95).

### Liposomal Delivery Vehicles

In some embodiments, a suitable delivery vehicle is a liposomal delivery vehicle, e.g., a lipid nanoparticle. As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). In the context of the present invention, a liposomal delivery vehicle typically serves to transport a desired nucleic acid (e.g., mRNA or MCNA) to a target cell or tissue. In some embodiments, a nanoparticle delivery vehicle is a liposome. In some embodiments, a liposome comprises one or more cationic lipids, one or more non-cationic lipids, one or more cholesterol-based lipids, or one or more PEG-modified lipids. In some embodiments, a liposome comprises no more than three distinct lipid components. In some embodiments, one distinct lipid component is a sterol-based cationic lipid.

### Cationic Lipids

As used herein, the phrase “cationic lipids” refers to any of a number of lipid species that have a net positive charge at a selected pH, such as physiological pH.

Suitable cationic lipids for use in the compositions and methods of the invention include the cationic lipids as described in International Patent Publication WO 2010/144740, which is incorporated herein by reference. In certain embodiments, the compositions and methods of the present invention include a cationic lipid, (6Z,9Z,28Z,31Z)-heptatriaconta-6,9,28,31-tetraen-19-yl 4-(dimethylamino) butanoate, having a compound structure of:

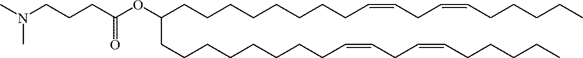 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the compositions and methods of the present invention include ionizable cationic lipids as described in International Patent Publication WO 2013/149140, which is incorporated herein by reference. In some embodiments, the compositions and methods of the present invention include a cationic lipid of one of the following formulas:

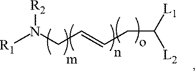

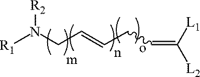

or a pharmaceutically acceptable salt thereof, wherein R₁and R₂are each independently selected from the group consisting of hydrogen, an optionally substituted, variably saturated or unsaturated C₁-C₂₀alkyl and an optionally substituted, variably saturated or unsaturated C₆-C₂₀ acyl; wherein L₁and L₂are each independently selected from the group consisting of hydrogen, an optionally substituted C₁-C₃₀alkyl, an optionally substituted variably unsaturated C₁-C₃₀ alkenyl, and an optionally substituted C₁-C₃₀alkynyl; wherein m and o are each independently selected from the group consisting of zero and any positive integer (e.g., where m is three); and wherein n is zero or any positive integer (e.g., where n is one). In certain embodiments, the compositions and methods of the present invention include the cationic lipid (15Z, 18Z)-N,N-dimethyl-6-(9Z,12Z)-octadeca-9,12-dien-1-yl) tetracosa-15,18-dien-1-amine (“HGT5000”), having a compound structure of:

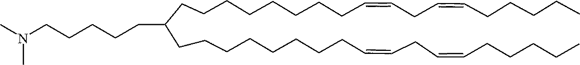 [SMILES: CCCCC/C=C\C/C=C\CCCCCCCCC(CCCCCCCC/C=C\C/C=C\CCCCC)CCCCCN(C)C]

and pharmaceutically acceptable salts thereof. In certain embodiments, the compositions and methods of the present invention include the cationic lipid (15Z, 18Z)-N,N-dimethyl-6-((9Z,12Z)-octadeca-9,12-dien-1-yl) tetracosa-4,15,18-trien-1-amine (“HGT5001”), having a compound structure of:

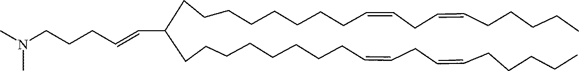 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In certain embodiments, the compositions and methods of the present invention include the cationic lipid and (15Z,18Z)-N,N-dimethyl-6-((9Z,12Z)-octadeca-9,12-dien-1-yl) tetracosa-5,15,18-trien- 1 -amine (“HGT5002”), having a compound structure of:

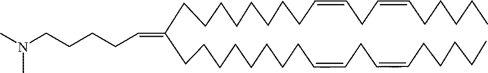 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the compositions and methods of the invention include cationic lipids described as aminoalcohol lipidoids in International Patent Publication WO 2010/053572, which is incorporated herein by reference. In certain embodiments, the compositions and methods of the present invention include a cationic lipid having a compound structure of:

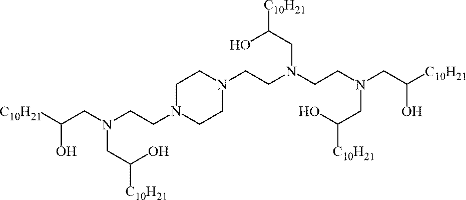

and pharmaceutically acceptable salts thereof.

Other suitable cationic lipids for use in the compositions and methods of the invention include the cationic lipids as described in International Patent Publication WO 2016/118725, which is incorporated herein by reference. In certain embodiments, the compositions and methods of the present invention include a cationic lipid having a compound structure of:

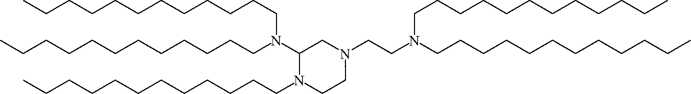 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the compositions and methods of the invention include the cationic lipids as described in International Patent Publication WO 2016/118724, which is incorporated herein by reference. In certain embodiments, the compositions and methods of the present invention include a cationic lipid having a compound structure of:

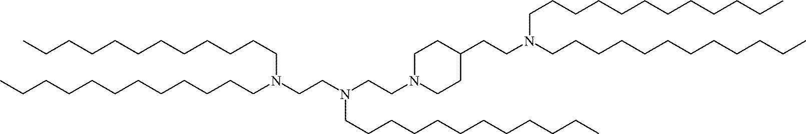 [SMILES: CCCCCCCCCCCCN(CCCCCCCCCCCC)CCC1CCN(CCN(CCCCCCCCCCCC)CCN(CCCCCCCCCCCC)CCCCCCCCCCCC)CC1]

and pharmaceutically acceptable salts thereof.

Other suitable cationic lipids for use in the compositions and methods of the invention include a cationic lipid having the formula of 14,25-ditridecyl 15,18,21,24-tetraaza-octatriacontane, and pharmaceutically acceptable salts thereof.

Other suitable cationic lipids for use in the compositions and methods of the invention include the cationic lipids as described in International Patent Publications WO 2013/063468 and WO 2016/205691, each of which are incorporated herein by reference. In some embodiments, the compositions and methods of the present invention include a cationic lipid of the following formula:

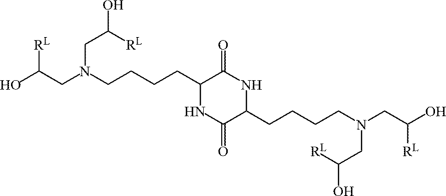

or pharmaceutically acceptable salts thereof, wherein each instance of R^{L}is independently optionally substituted C₆-C₄₀alkenyl. In certain embodiments, the compositions and methods of the present invention include a cationic lipid having a compound structure of:

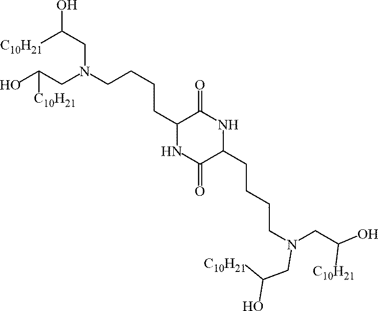

and pharmaceutically acceptable salts thereof. In certain embodiments, the compositions and methods of the present invention include a cationic lipid having a compound structure of:

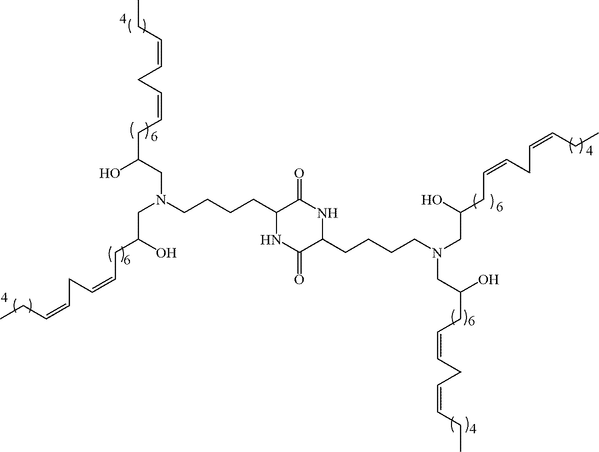

and pharmaceutically acceptable salts thereof. In certain embodiments, the compositions and methods of the present invention include a cationic lipid having a compound structure of:

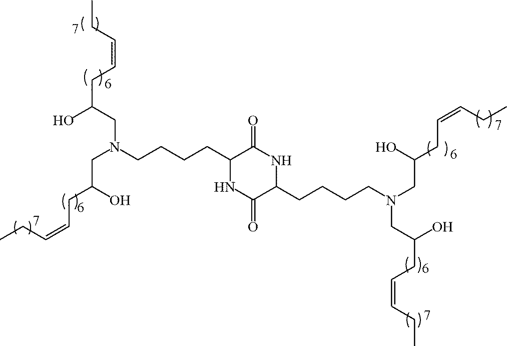

and pharmaceutically acceptable salts thereof. In certain embodiments, the compositions and methods of the present invention include a cationic lipid having a compound structure of:

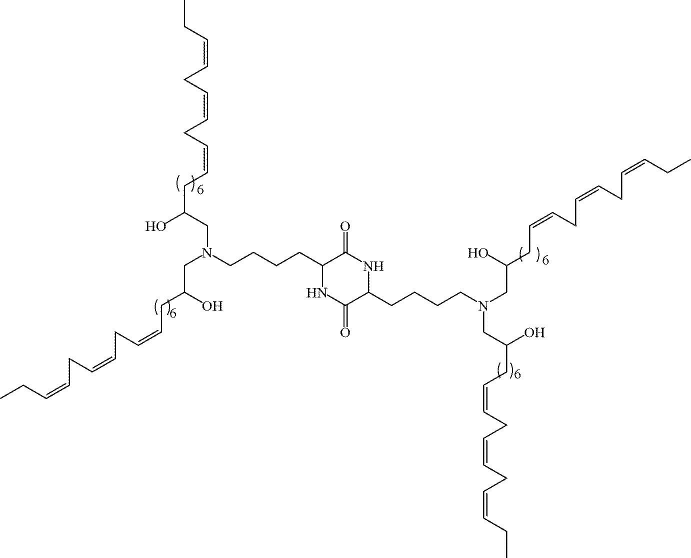

and pharmaceutically acceptable salts thereof.

Other suitable cationic lipids for use in the compositions and methods of the invention include the cationic lipids as described in International Patent Publication WO 2015/184256, which is incorporated herein by reference. In some embodiments, the compositions and methods of the present invention include a cationic lipid of the following formula:

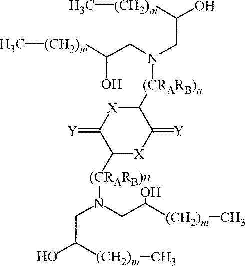

or a pharmaceutically acceptable salt thereof, wherein each X independently is O or S; each Y independently is O or S; each m independently is 0 to 20; each n independently is 1 to 6; each R_{A}is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each R_{B}is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In certain embodiments, the compositions and methods of the present invention include a cationic lipid, “Target 23”, having a compound structure of:

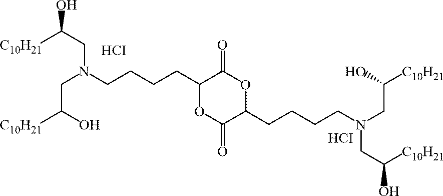

and pharmaceutically acceptable salts thereof.

Other suitable cationic lipids for use in the compositions and methods of the invention include the cationic lipids as described in International Patent Publication WO 2016/004202, which is incorporated herein by reference. In some embodiments, the compositions and methods of the present invention include a cationic lipid having the compound structure:

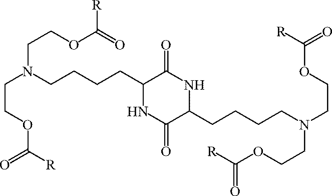

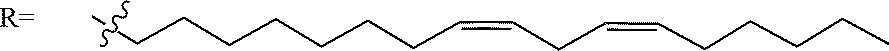

or a pharmaceutically acceptable salt thereof. In some embodiments, the compositions and methods of the present invention include a cationic lipid having the compound structure:

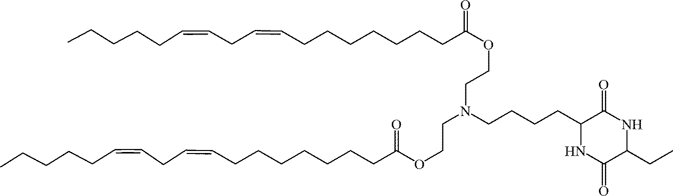 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the compositions and methods of the present invention include a cationic lipid having the compound structure:

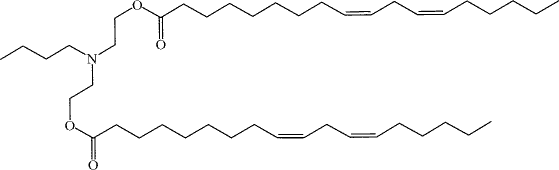 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the compositions and methods of the present invention include cationic lipids as described in U.S. Provisional Pat. Application Serial No. 62/758,179, which is incorporated herein by reference. In some embodiments, the compositions and methods of the present invention include a cationic lipid of the following formula:

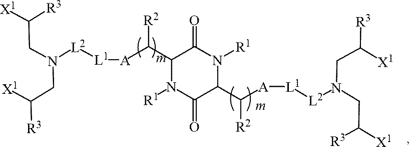

or a pharmaceutically acceptable salt thereof, wherein each R¹and R²is independently H or C₁-C₆aliphatic; each m is independently an integer having a value of 1 to 4; each A is independently a covalent bond or arylene; each L¹is independently an ester, thioester, disulfide, or anhydride group; each L²is independently C₂-C₁₀aliphatic; each X¹is independently H or OH; and each R³is independently C₆-C₂₀aliphatic. In some embodiments, the compositions and methods of the present invention include a cationic lipid of the following formula:

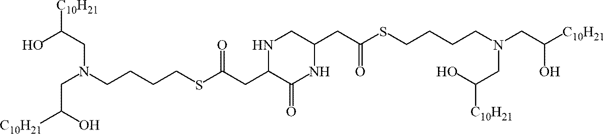

or a pharmaceutically acceptable salt thereof. In some embodiments, the compositions and methods of the present invention include a cationic lipid of the following formula:

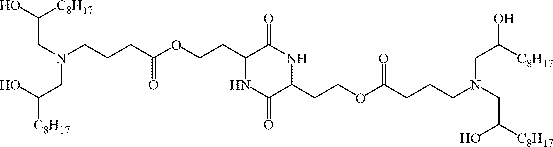

or a pharmaceutically acceptable salt thereof. In some embodiments, the compositions and methods of the present invention include a cationic lipid of the following formula:

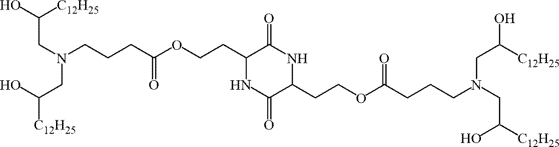

or a pharmaceutically acceptable salt thereof.

Other suitable cationic lipids for use in the compositions and methods of the present invention include the cationic lipids as described in J. McClellan, M. C. King, Cell 2010, 141, 210-217 and in Whitehead et al. , Nature Communications (2014) 5:4277, which is incorporated herein by reference. In certain embodiments, the cationic lipids of the compositions and methods of the present invention include a cationic lipid having a compound structure of:

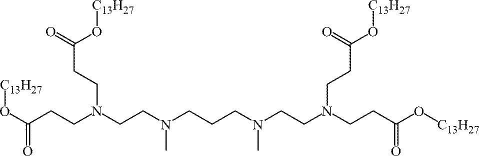

and pharmaceutically acceptable salts thereof.

Other suitable cationic lipids for use in the compositions and methods of the invention include the cationic lipids as described in International Patent Publication WO 2015/199952, which is incorporated herein by reference. In some embodiments, the compositions and methods of the present invention include a cationic lipid having the compound structure:

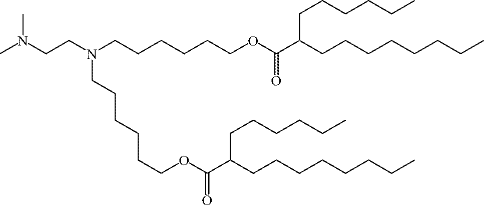 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

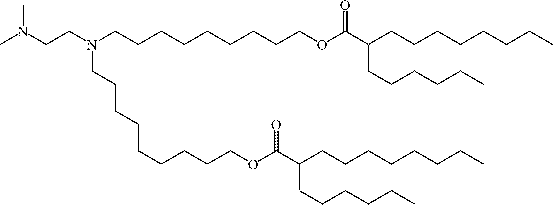 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

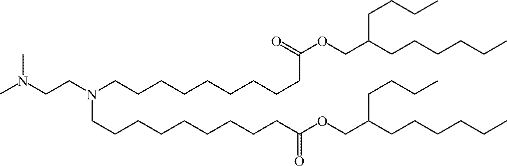 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the compositions and methods of the present invention include a cationic lipid having the compound structure:

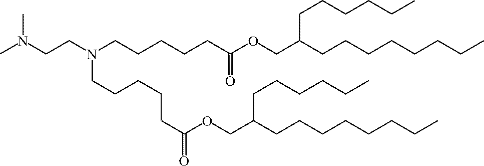 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

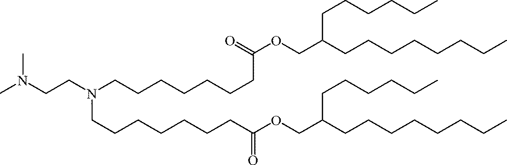 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

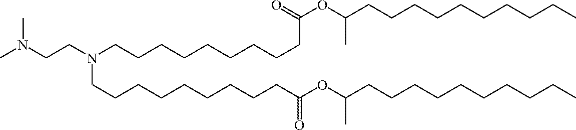 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof. In some embodiments, the compositions and methods of the present invention include a cationic lipid having the compound structure:

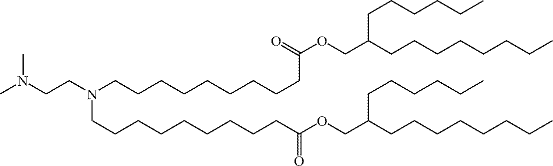 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

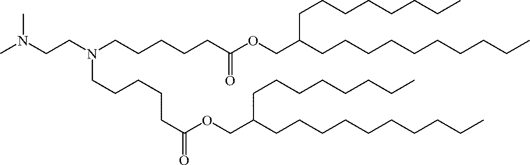 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

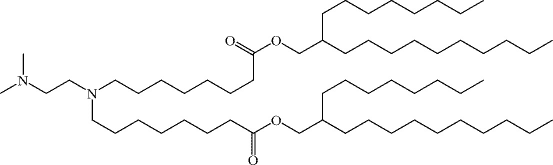 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

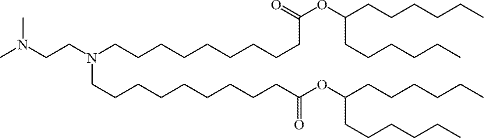 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the compositions and methods of the present invention include a cationic lipid having the compound structure:

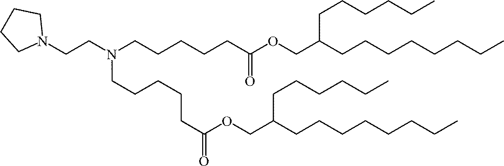 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the compositions and methods of the present invention include a cationic lipid having the compound structure:

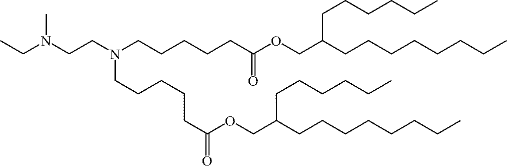 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the compositions and methods of the present invention include a cationic lipid having the compound structure:

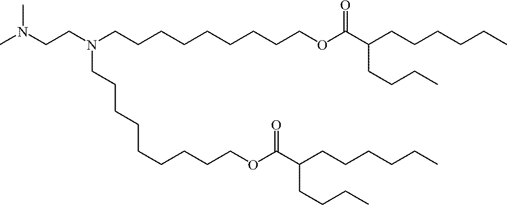 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the compositions and methods of the invention include the cationic lipids as described in International Patent Publication WO 2017/004143, which is incorporated herein by reference. In some embodiments, the compositions and methods of the present invention include a cationic lipid having the compound structure:

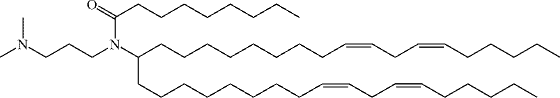 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the compositions and methods of the present invention include a cationic lipid having the compound structure:

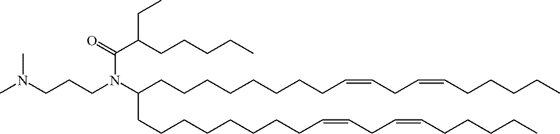 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the compositions and methods of the present invention include a cationic lipid having the compound structure:

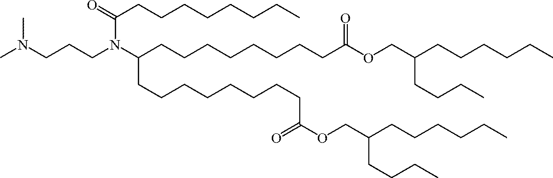 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

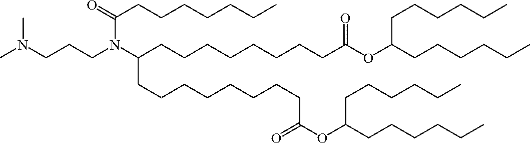 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

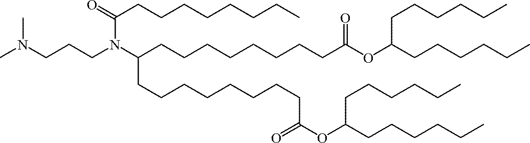 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

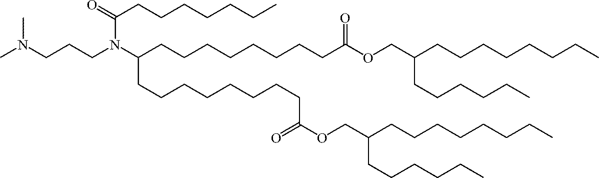 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

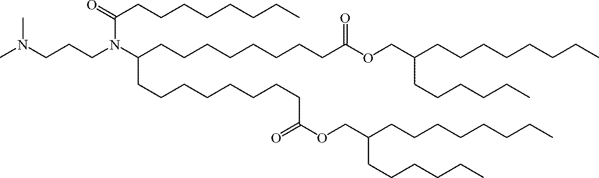 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the compositions and methods of the present invention include a cationic lipid having the compound structure:

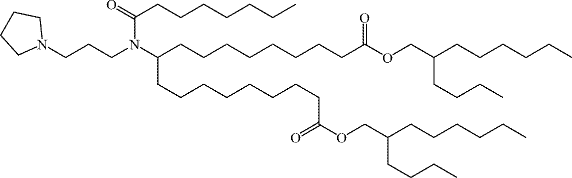 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

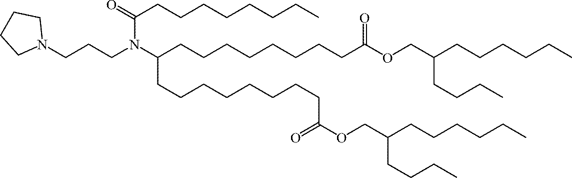 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

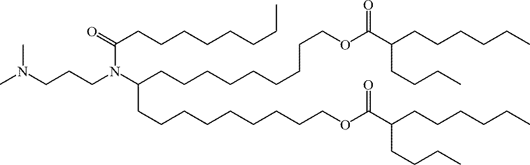 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the compositions and methods of the present invention include a cationic lipid having the compound structure:

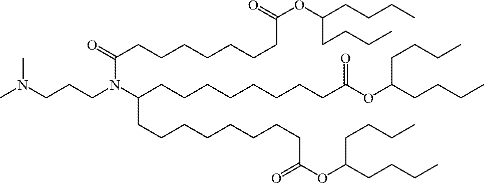 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the compositions and methods of the present invention include a cationic lipid having the compound structure:

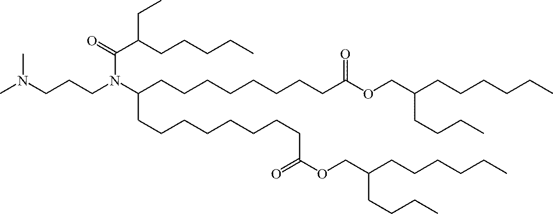 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the compositions and methods of the present invention include a cationic lipid having the compound structure:

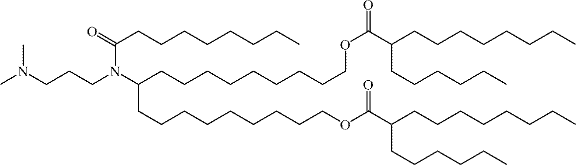 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the compositions and methods of the present invention include a cationic lipid having the compound structure:

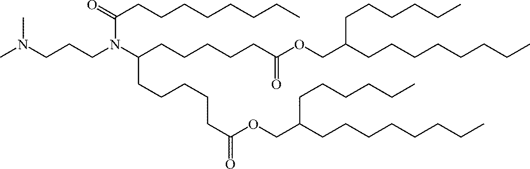 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the compositions and methods of the present invention include a cationic lipid having the compound structure:

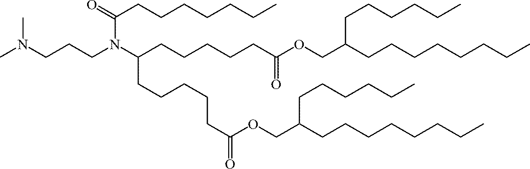 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

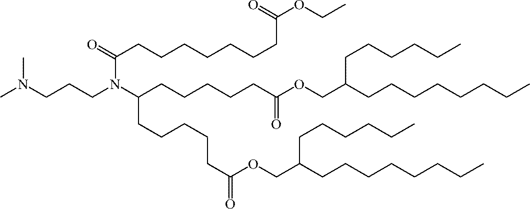 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the compositions and methods of the present invention include a cationic lipid having the compound structure:

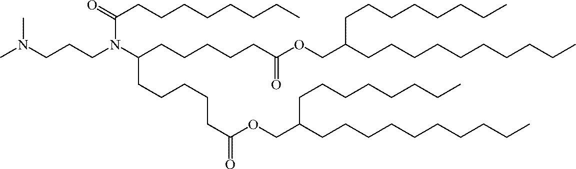 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the compositions and methods of the invention include the cationic lipids as described in International Patent Publication WO 2017/075531, which is incorporated herein by reference. In some embodiments, the compositions and methods of the present invention include a cationic lipid of the following formula:

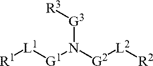

or a pharmaceutically acceptable salt thereof, wherein one of L¹or L²is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹or L²is —O(C═O)—, —(C═O)O—, —C(═O)—, —O—, —S(O) ₓ, —S—S—, —C(═O)S—, SC(═O)—, —NR^{a}C(═O)—, —C(═O)NR^{a}—, ,NR^{a}C(═O)NR^{a}—, —OC(═O)NR^{a}— or —NR^{a}C(═O)O— or a direct bond; G¹and G²are each independently unsubstituted C₁-C₁₂alkylene or C₁-C₁₂alkenylene; G³is C₁-C₂₄alkylene, C₁-C₂₄alkenylene, C₃-C₈cycloalkylene, C₃-C₈cycloalkenylene; R^{a}is H or C₁-C₁₂alkyl; R¹and R²are each independently C₆-C₂₄alkyl or C₆-C₂₄alkenyl; R³is H, OR⁵, CN, —C(═O)OR⁴, —OC(═O)R⁴or —NR⁵C(═O)R⁴; R⁴is C₁-C₁₂alkyl; R⁵ is H or C₁-C₆alkyl; and x is 0, 1 or 2.

Other suitable cationic lipids for use in the compositions and methods of the invention include the cationic lipids as described in International Patent Publication WO 2017/117528, which is incorporated herein by reference. In some embodiments, the compositions and methods of the present invention include a cationic lipid having the compound structure:

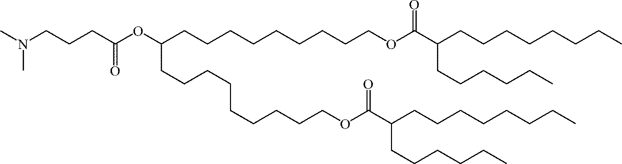 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the compositions and methods of the present invention include a cationic lipid having the compound structure:

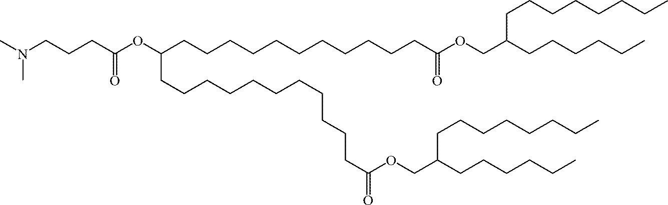 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the compositions and methods of the present invention include a cationic lipid having the compound structure:

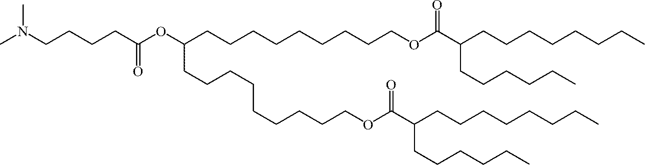 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the compositions and methods of the invention include the cationic lipids as described in International Patent Publication WO 2017/049245, which is incorporated herein by reference. In some embodiments, the cationic lipids of the compositions and methods of the present invention include a compound of one of the following formulas:

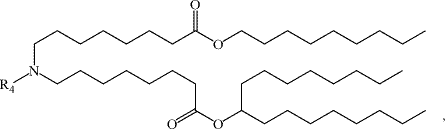

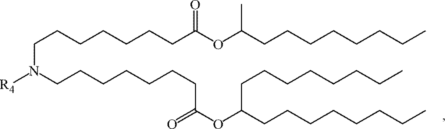

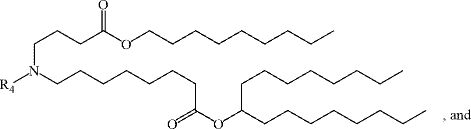

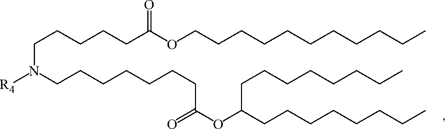

and pharmaceutically acceptable salts thereof. For any one of these four formulas, R₄is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3. In certain embodiments, the compositions and methods of the present invention include a cationic lipid having a compound structure of:

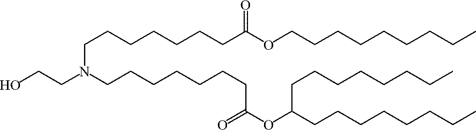 [SMILES: CCCCCCCCCOC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

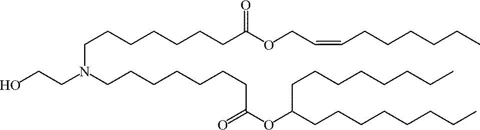 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

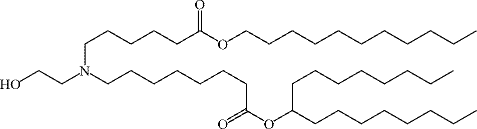 [SMILES: CCCCCCCCCCCOC(=O)CCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

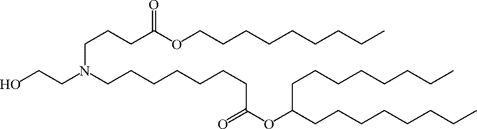 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the compositions and methods of the invention include the cationic lipids as described in International Patent Publication WO 2017/173054 and WO 2015/095340, each of which is incorporated herein by reference. In certain embodiments, the compositions and methods of the present invention include a cationic lipid having a compound structure of:

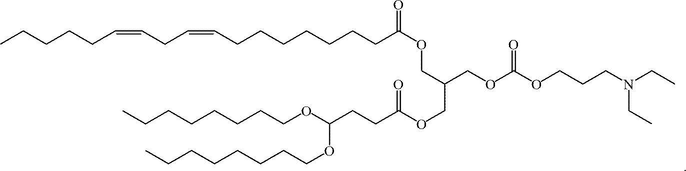

and pharmaceutically acceptable salts thereof. In certain embodiments, the compositions and methods of the present invention include a cationic lipid having a compound structure of:

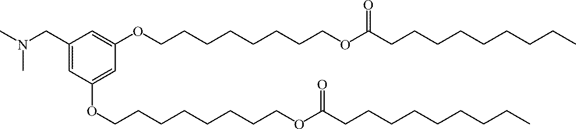 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In certain embodiments, the compositions and methods of the present invention include a cationic lipid having a compound structure of:

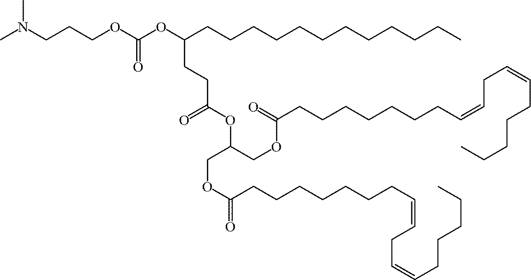 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In certain embodiments, the compositions and methods of the present invention include a cationic lipid having a compound structure of:

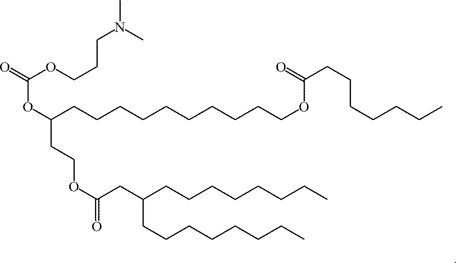

and pharmaceutically acceptable salts thereof.

Other suitable cationic lipids for use in the compositions and methods of the present invention include cleavable cationic lipids as described in International Patent Publication WO 2012/170889, which is incorporated herein by reference. In some embodiments, the compositions and methods of the present invention include a cationic lipid of the following formula:

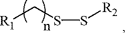

wherein R₁is selected from the group consisting of imidazole, guanidinium, amino, imine, enamine, an optionally-substituted alkyl amino (e.g., an alkyl amino such as dimethylamino) and pyridyl; wherein R₂is selected from the group consisting of one of the following two formulas:

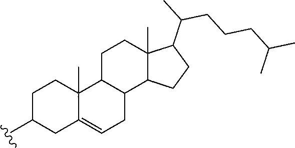

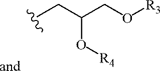

and wherein R₃and R₄are each independently selected from the group consisting of an optionally substituted, variably saturated or unsaturated C₆-C₂₀alkyl and an optionally substituted, variably saturated or unsaturated C₆-C₂₀acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In certain embodiments, the compositions and methods of the present invention include a cationic lipid, “HGT4001”, having a compound structure of:

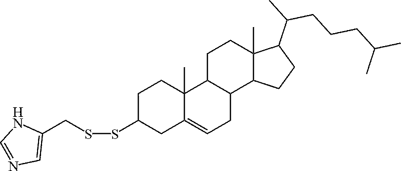 [SMILES: CC(C)CCCC(C)C1CCC2C3CC=C4CC(SSCc5cnc[nH]5)CCC4(C)C3CCC12C]

and pharmaceutically acceptable salts thereof. In certain embodiments, the compositions and methods of the present invention include a cationic lipid, “HGT4002,” (also referred to herein as “Guan-SS-Chol”) having a compound structure of:

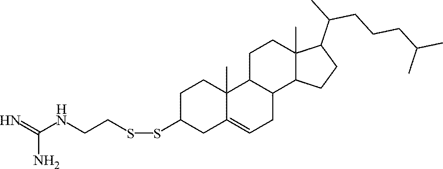 [SMILES: CC(C)CCCC(C)C1CCC2C3CC=C4CC(SSCCNC(=N)N)CCC4(C)C3CCC12C]

and pharmaceutically acceptable salts thereof. In certain embodiments, the compositions and methods of the present invention include a cationic lipid, “HGT4003,” having a compound structure of:

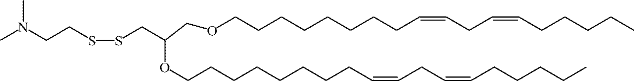 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In certain embodiments, the compositions and methods of the present invention include a cationic lipid, “HGT4004,” having a compound structure of:

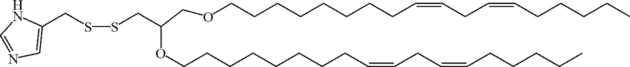 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In certain embodiments, the compositions and methods of the present invention include a cationic lipid “HGT4005,” having a compound structure of:

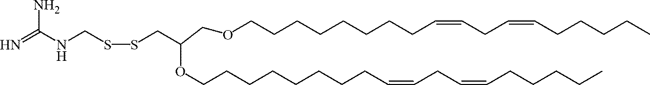 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Other suitable cationic lipids for use in the compositions and methods of the present invention include cleavable cationic lipids as described in International Application No. PCT/US2019/032522, and incorporated herein by reference. In certain embodiments, the compositions and methods of the present invention include a cationic lipid that is any of general formulas or any of structures (1a)-(21a) and (1b) - (21b) and (22)-(237) described in International Application No. PCT/US2019/032522. In certain embodiments, the compositions and methods of the present invention include a cationic lipid that has a structure according to Formula (I′),

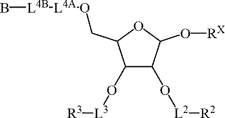

wherein:
R^{X}is independently —H, —L¹—R¹, or —L^{5A}—L^{5B}—B′;
each of L¹, L², and L³is independently a covalent bond, —C(O)—, —C(O)O—, —C(O)S—, or —C(O)NR^{L}—;
each L^{4A}and L^{5A}is independently —C(O)—, —C(O)O—, or —C(O)NR^{L}—;
each L^{4B}and L^{5B}is independently C₁-C₂₀alkylene; C₂-C₂₀alkenylene; or C₂-C₂₀ alkynylene;
each B and B′ is NR⁴R⁵or a 5- to 10-membered nitrogen-containing heteroaryl;
each R¹, R², and R³is independently C₆-C₃₀alkyl, C₆-C₃₀alkenyl, or C₆-C₃₀alkynyl;
each R⁴and R⁵is independently hydrogen, C₁-C₁₀alkyl; C₂-C₁₀alkenyl; or C₂-C₁₀ alkynyl; and
each R^{L}is independently hydrogen, C₁-C₂₀alkyl, C₂-C₂₀alkenyl, or C₂-C₂₀alkynyl.

In certain embodiments, the compositions and methods of the present invention include a cationic lipid that is Compound (139) of International Application No. PCT/US2019/032522, having a compound structure of:

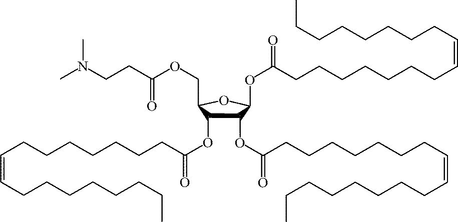

(“18:1 Carbon tail-ribose lipid”).

In some embodiments, the compositions and methods of the present invention include a cationic lipid that is TL1-04D-DMA, having a compound structure of:

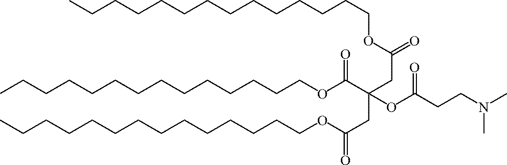 [SMILES: CCCCCCCCCCCCCCOC(=O)CC(CC(=O)OCCCCCCCCCCCCCC)(OC(=O)CCN(C)C)C(=O)OCCCCCCCCCCCCCC]

(“TL1-04D-DMA”).

In some embodiments, the compositions and methods of the present invention include a cationic lipid that is GL-TES-SA-DME-E18-2, having a compound structure of:

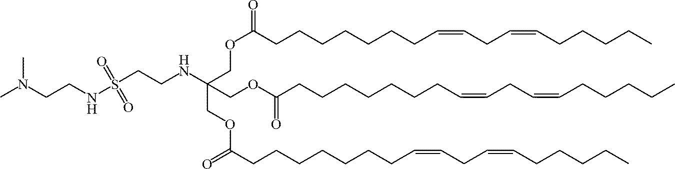 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCN(C)C]

(“GL-TES-SA-DME-E18-2”).

In some embodiments, the compositions and methods of the present invention include a cationic lipid that is Guan-SS-Chol, having a compound structure of:

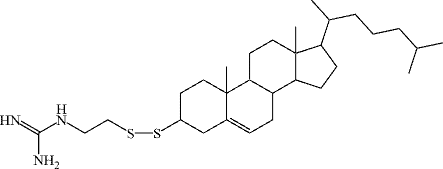 [SMILES: CC(C)CCCC(C)C1CCC2C3CC=C4CC(SSCCNC(=N)N)CCC4(C)C3CCC12C]

(“Guan-S S-Chol”).

In some embodiments, the compositions and methods of the present invention include a cationic lipid that is SY-3-E14-DMAPr, having a compound structure of:

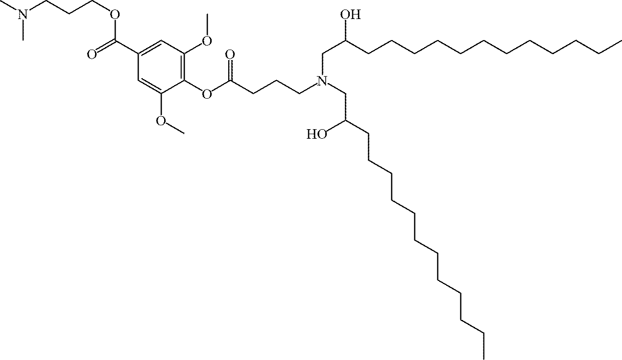 [SMILES: CCCCCCCCCCCCC(O)CN(CCCC(=O)Oc1c(OC)cc(C(=O)OCCCN(C)C)cc1OC)CC(O)CCCCCCCCCCCC]

(“SY-3-E14-DMAPr”).

In some embodiments, the compositions and methods of the present invention include a cationic lipid that is RL3-07D-DMA, having a compound structure of:

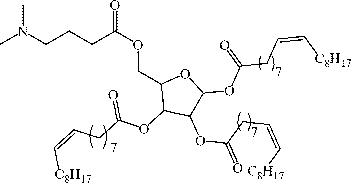

(“RL3-07D-DMA”).

In some embodiments, the compositions and methods of the present invention include a cationic lipid that is TL1-01D-DMA, having a compound structure of:

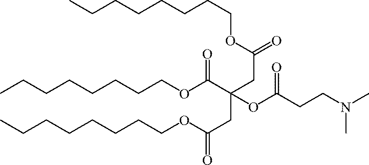 [SMILES: CCCCCCCCOC(=O)CC(CC(=O)OCCCCCCCC)(OC(=O)CCN(C)C)C(=O)OCCCCCCCC]

(“TL1-01D-DMA”).

In some embodiments, the compositions and methods of the present invention include the cationic lipid, N-[1-(2,3-dioleyloxy)propyl]-N,N,N-trimethylammonium chloride (“DOTMA”). (Feigner et al. (Proc. Nat′l Acad. Sci. 84, 7413 (1987); U.S. Pat. No. 4,897,355, which is incorporated herein by reference). Other cationic lipids suitable for the compositions and methods of the present invention include, for example, 5-carboxyspermylglycinedioctadecylamide (“DOGS”); 2,3-dioleyloxy-N-[2(spermine-carboxamido)ethyl]-N,N-dimethyl-1-propanaminium (“DOSPA”) (Behr et al. Proc. Nat.’l Acad. Sci. 86, 6982 (1989), U.S. Pat. No. 5,171,678; U.S. Pat. No. 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the compositions and methods of the present invention also include: 1,2-distearyloxy-N,N-dimethyl-3-aminopropane ( “DSDMA”); 1,2-dioleyloxy-N,N-dimethyl-3-aminopropane (“DODMA”); 1 ,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-[5′-(cholest-5-en-3-beta-oxy)-3′-oxapentoxy)-3-dimethy 1-1-(cis,cis-9′, 1-2′-octadecadienoxy)propane (“CpLinDMA”); N,N-dimethyl-3,4-dioleyloxybenzylamine (“DMOBA”); 1 ,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxy]octyl)oxy)-N, N-dimethyl-3-[(9Z, 12Z)-octadeca-9, 12-dien-1 -yloxy]propane-1-amine (“Octyl-CLinDMA”); (2R)-2-((8-[(3beta)-cholest-5-en-3-yloxy]octyl)oxy)-N, N-dimethyl-3-[(9Z, 12Z)-octadeca-9, 12-dien-1-yloxy]propan-1 -amine (“Octyl-CLinDMA (2R)”); (2S)-2-((8-[(3P)-cholest-5-en-3-yloxy]octyl)oxy)-N, fsl-dimethyh3-[(9Z, 12Z)-octadeca-9, 12-dien-1 -yloxy]propan-1 -amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,12-dien- 1-yl)-1 ,3-dioxolan-4-yl)-N,N-dimethylethanamine (“DLin-KC2-DMA”) (see, WO 2010/042877, which is incorporated herein by reference; Semple et al. , Nature Biotech. 28: 172-176 (2010)). (Heyes, J., et al. , J Controlled Release 107: 276-287 (2005); Morrissey, DV., et al. , Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety.

In some embodiments, one or more cationic lipids suitable for the compositions and methods of the present invention include 2,2-Dilinoleyl-4-dimethylaminoethyl-[1,3]-dioxolane (“XTC”); (3aR,5s,6aS)-N,N-dimethyl-2,2-di((9Z,12Z)-octadeca-9,12-dienyl)tetrahydro-3aH-cyclopenta[d] [1 ,3]dioxol-5-amine (“ALNY-100”) and/or 4,7,13-tris(3-oxo-3-(undecylamino)propyl)-N1,N16-diundecyl-4,7,10,13-tetraazahexadecane-1,16-diamide (“NC98-5”).

In some embodiments, one or more cationic lipids suitable for the compositions and methods of the present invention include a cationic lipid that is TL1-04D-DMA, having a compound structure of:

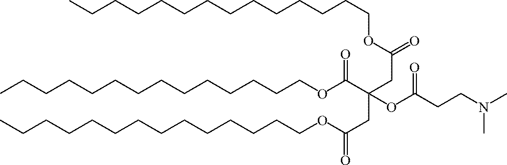 [SMILES: CCCCCCCCCCCCCCOC(=O)CC(CC(=O)OCCCCCCCCCCCCCC)(OC(=O)CCN(C)C)C(=O)OCCCCCCCCCCCCCC]

(“TL1-04D-DMA”). In some embodiments, one or more cationic lipids suitable for the compositions and methods of the present invention include a cationic lipid that is GL-TES-SA-DME-E18-2, having a compound structure of:

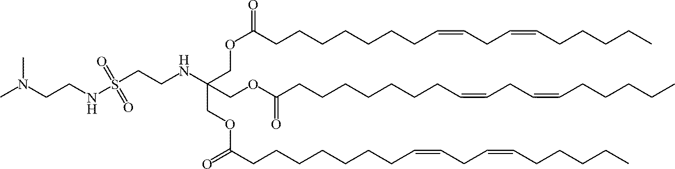 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCN(C)C]

(“GL-TES-SA-DME-E18-2”). In some embodiments, one or more cationic lipids suitable for the compositions and methods of the present invention include a cationic lipid that is SY-3-E14-DMAPr, having a compound structure of:

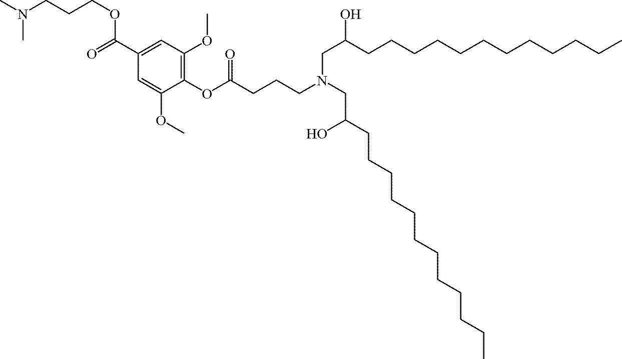 [SMILES: CCCCCCCCCCCCC(O)CN(CCCC(=O)Oc1c(OC)cc(C(=O)OCCCN(C)C)cc1OC)CC(O)CCCCCCCCCCCC]

(“SY-3-E14-DMAPr”). In some embodiments, one or more cationic lipids suitable for the compositions and methods of the present invention include a cationic lipid that is TL1-01D-DMA, having a compound structure of:

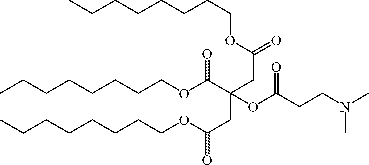 [SMILES: CCCCCCCCOC(=O)CC(CC(=O)OCCCCCCCC)(OC(=O)CCN(C)C)C(=O)OCCCCCCCC]

(“TL1-01D-DMA”). In some embodiments, one or more cationic lipids suitable for the compositions and methods of the present invention include a cationic lipid that is TL1-10D-DMA, having a compound structure of:

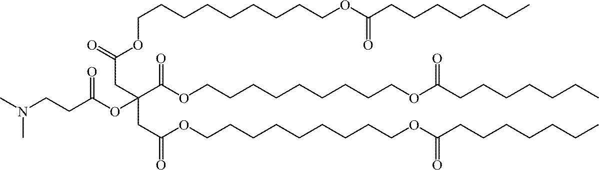 [SMILES: CCCCCCCC(=O)OCCCCCCCCCOC(=O)CC(CC(=O)OCCCCCCCCCOC(=O)CCCCCCC)(OC(=O)CCN(C)C)C(=O)OCCCCCCCCCOC(=O)CCCCCCC]

(“TL1-10D-DMA”).

In some embodiments, one or more cationic lipids suitable for the compositions and methods of the present invention include a cationic lipid that is GL-TES-SA-DMP-E18-2, having a compound structure of:

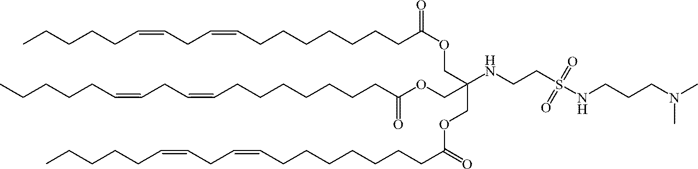 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCCN(C)C]

(“GL-TES-SA-DMP-E 18-2”).

In some embodiments, one or more cationic lipids suitable for the compositions and methods of the present invention include a cationic lipid that is HEP-E4-E10, having a compound structure of:

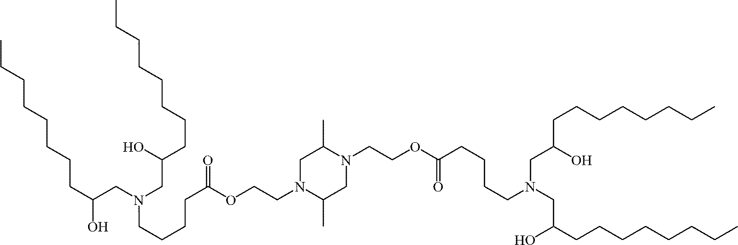 [SMILES: CCCCCCCCC(O)CN(CCCCC(=O)OCCN1CC(C)N(CCOC(=O)CCCCN(CC(O)CCCCCCCC)CC(O)CCCCCCCC)CC1C)CC(O)CCCCCCCC]

(“HEP-E4-E10”). In some embodiments, one or more cationic lipids suitable for the compositions and methods of the present invention include a cationic lipid that is HEP-E3-E10, having a compound structure of:

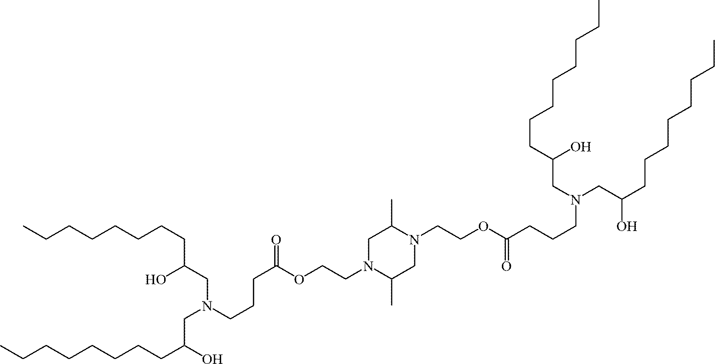 [SMILES: CCCCCCCCC(O)CN(CCCC(=O)OCCN1CC(C)N(CCOC(=O)CCCN(CC(O)CCCCCCCC)CC(O)CCCCCCCC)CC1C)CC(O)CCCCCCCC]

(“HEP-E3-E10”).

In some embodiments, the compositions of the present invention include one or more cationic lipids that constitute at least about 5%, 10%, 20%, 30%, 35%, 40%, 45%, 50%, 55%, 60%, 65%, or 70%, measured by weight, of the total lipid content in the composition, e.g., a lipid nanoparticle. In some embodiments, the compositions of the present invention include one or more cationic lipids that constitute at least about 5%, 10%, 20%, 30%, 35%, 40%, 45%, 50%, 55%, 60%, 65%, or 70%, measured as a mol %, of the total lipid content in the composition, e.g., a lipid nanoparticle. In some embodiments, the compositions of the present invention include one or more cationic lipids that constitute about 30-70 % (e.g., about 30-65%, about 30-60%, about 30-55%, about 30-50%, about 30-45%, about 30-40%, about 35-50%, about 35-45%, or about 35-40%), measured by weight, of the total lipid content in the composition, e.g., a lipid nanoparticle. In some embodiments, the compositions of the present invention include one or more cationic lipids that constitute about 30-70 % (e.g., about 30-65%, about 30-60%, about 30-55%, about 30-50%, about 30-45%, about 30-40%, about 35-50%, about 35-45%, or about 35-40%), measured as mol %, of the total lipid content in the composition, e.g., a lipid nanoparticle.

### Non-Cationic/Helper Lipids

In some embodiments, the liposomes contain one or more non-cationic (“helper”) lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), phosphatidylserine, sphingolipids, cerebrosides, gangliosides, 16-O-monomethyl PE, 16-0-dimethyl PE, 18-1-trans PE, 1-stearoyl-2-oleoyl-phosphatidyethanolamine (SOPE), or a mixture thereof.

In some embodiments, a non-cationic lipid is a neutral lipid, i.e., a lipid that does not carry a net charge in the conditions under which the composition is formulated and/or administered.

In some embodiments, such non-cationic lipids may be used alone, but are preferably used in combination with other lipids, for example, cationic lipids.

In some embodiments, a non-cationic lipid may be present in a molar ratio (mol%) of about 5% to about 90%, about 5% to about 70%, about 5% to about 50%, about 5% to about 40%, about 5% to about 30%, about 10 % to about 70%, about 10% to about 50%, or about 10% to about 40% of the total lipids present in a composition. In some embodiments, total non-cationic lipids may be present in a molar ratio (mol%) of about 5% to about 90%, about 5% to about 70%, about 5% to about 50%, about 5% to about 40%, about 5% to about 30%, about 10 % to about 70%, about 10% to about 50%, or about 10% to about 40% of the total lipids present in a composition. In some embodiments, the percentage of non-cationic lipid in a liposome may be greater than about 5 mol%, greater than about 10 mol%, greater than about 20 mol%, greater than about 30 mol%, or greater than about 40 mol%. In some embodiments, the percentage total non-cationic lipids in a liposome may be greater than about 5 mol%, greater than about 10 mol%, greater than about 20 mol%, greater than about 30 mol%, or greater than about 40 mol%. In some embodiments, the percentage of non-cationic lipid in a liposome is no more than about 5 mol%, no more than about 10 mol%, no more than about 20 mol%, no more than about 30 mol%, or no more than about 40 mol%. In some embodiments, the percentage total non-cationic lipids in a liposome may be no more than about 5 mol%, no more than about 10 mol%, no more than about 20 mol%, no more than about 30 mol%, or no more than about 40 mol%.

In some embodiments, a non-cationic lipid may be present in a weight ratio (wt%) of about 5% to about 90%, about 5% to about 70%, about 5% to about 50%, about 5% to about 40%, about 5% to about 30%, about 10 % to about 70%, about 10% to about 50%, or about 10% to about 40% of the total lipids present in a composition. In some embodiments, total non-cationic lipids may be present in a weight ratio (wt%) of about 5% to about 90%, about 5% to about 70%, about 5% to about 50%, about 5% to about 40%, about 5% to about 30%, about 10 % to about 70%, about 10% to about 50%, or about 10% to about 40% of the total lipids present in a composition. In some embodiments, the percentage of non-cationic lipid in a liposome may be greater than about 5 wt%, greater than about 10 wt%, greater than about 20 wt%, greater than about 30 wt%, or greater than about 40 wt%. In some embodiments, the percentage total non-cationic lipids in a liposome may be greater than about 5 wt%, greater than about 10 wt%, greater than about 20 wt%, greater than about 30 wt%, or greater than about 40 wt%. In some embodiments, the percentage of non-cationic lipid in a liposome is no more than about 5 wt%, no more than about 10 wt%, no more than about 20 wt%, no more than about 30 wt%, or no more than about 40 wt%. In some embodiments, the percentage total non-cationic lipids in a liposome may be no more than about 5 wt%, no more than about 10 wt%, no more than about 20 wt%, no more than about 30 wt%, or no more than about 40 wt%.

### Cholesterol-Based Lipids

In some embodiments, the liposomes comprise one or more cholesterol-based lipids. For example, suitable cholesterol-based cationic lipids include, for example, DC-Choi (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or imidazole cholesterol ester (ICE) , which has the following structure,

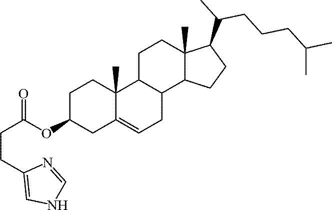 [SMILES: CC(C)CCCC(C)[C@H]1CCC2C3CC=C4C[C@@H](OC(=O)CCc5c[nH]cn5)CC[C@]4(C)C3CC[C@@]21C]

(“ICE”).

In embodiments, a cholesterol-based lipid is cholesterol.

In some embodiments, the cholesterol-based lipid may comprise a molar ratio (mol%) of about 1% to about 30%, or about 5% to about 20% of the total lipids present in a liposome. In some embodiments, the percentage of cholesterol-based lipid in the lipid nanoparticle may be greater than about 5 mol%, greater than about 10 mol%, greater than about 20 mol%, greater than about 30 mol%, or greater than about 40 mol%. In some embodiments, the percentage of cholesterol-based lipid in the lipid nanoparticle may be no more than about 5 mol%, no more than about 10 mol%, no more than about 20 mol%, no more than about 30 mol%, or no more than about 40 mol%.

In some embodiments, a cholesterol-based lipid may be present in a weight ratio (wt%) of about 1% to about 30%, or about 5% to about 20% of the total lipids present in a liposome. In some embodiments, the percentage of cholesterol-based lipid in the lipid nanoparticle may be greater than about 5 wt%, greater than about 10 wt%, greater than about 20 wt%, greater than about 30 wt%, or greater than about 40 wt%. In some embodiments, the percentage of cholesterol-based lipid in the lipid nanoparticle may be no more than about 5 wt%, no more than about 10 wt%, no more than about 20 wt%, no more than about 30 wt%, or no more than about 40 wt%.

### PEG-Modified Lipids

In some embodiments, the liposome comprises one or more PEGylated lipids.

For example, the use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipid formulations together which comprise the transfer vehicle (e.g., a lipid nanoparticle).

Contemplated PEG-modified lipids include, but are not limited to, a polyethylene glycol chain of up to 5 kDa in length covalently attached to a lipid with alkyl chain(s) of C₆-C₂₀length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C₁₄or C₁₈).

The PEG-modified phospholipid and derivitized lipids of the present invention may comprise a molar ratio from about 0% to about 20%, about 0.5% to about 20%, about 1% to about 15%, about 4% to about 10%, or about 2% of the total lipid present in the liposomal transfer vehicle. In some embodiments, one or more PEG-modified lipids constitute about 4% of the total lipids by molar ratio. In some embodiments, one or more PEG-modified lipids constitute about 5% of the total lipids by molar ratio. In some embodiments, one or more PEG-modified lipids constitute about 6% of the total lipids by molar ratio.

### Amphiphilic Block Copolymers

In some embodiments, a suitable delivery vehicle contains amphiphilic block copolymers (e.g., poloxamers).

Various amphiphilic block copolymers may be used to practice the present invention. In some embodiments, an amphiphilic block copolymer is also referred to as a surfactant or a non-ionic surfactant.

In some embodiments, an amphiphilic polymer suitable for the invention is selected from poloxamers (Pluronic®), poloxamines (Tetronic®), polyoxyethylene glycol sorbitan alkyl esters (polysorbates) and polyvinyl pyrrolidones (PVPs).

### Poloxamers

In some embodiments, a suitable amphiphilic polymer is a poloxamer. For example, a suitable poloxamer is of the following structure:

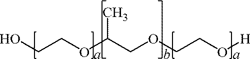

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

In some embodiments, a poloxamer suitable for the invention has ethylene oxide units from about 10 to about 150. In some embodiments, a poloxamer has ethylene oxide units from about 10 to about 100.

In some embodiments, a suitable poloxamer is poloxamer 84. In some embodiments, a suitable poloxamer is poloxamer 101. In some embodiments, a suitable poloxamer is poloxamer 105. In some embodiments, a suitable poloxamer is poloxamer 108. In some embodiments, a suitable poloxamer is poloxamer 122. In some embodiments, t a suitable poloxamer is poloxamer 123. In some embodiments, a suitable poloxamer is poloxamer 124. In some embodiments, a suitable poloxamer is poloxamer 181. In some embodiments, a suitable poloxamer is poloxamer 182. In some embodiments, a suitable poloxamer is poloxamer 183. In some embodiments, a suitable poloxamer is poloxamer 184. In some embodiments, a suitable poloxamer is poloxamer 185. In some embodiments, a suitable poloxamer is poloxamer 188. In some embodiments, a suitable poloxamer is poloxamer 212. In some embodiments, a suitable poloxamer is poloxamer 215. In some embodiments, a suitable poloxamer is poloxamer 217. In some embodiments, a suitable poloxamer is poloxamer 231. In some embodiments, a suitable poloxamer is poloxamer 234. In some embodiments, a suitable poloxamer is poloxamer 235. In some embodiments, a suitable poloxamer is poloxamer 237. In some embodiments, a suitable poloxamer is poloxamer 238. In some embodiments, a suitable poloxamer is poloxamer 282. In some embodiments, a suitable poloxamer is poloxamer 284. In some embodiments, a suitable poloxamer is poloxamer 288. In some embodiments, a suitable poloxamer is poloxamer 304. In some embodiments, a suitable poloxamer is poloxamer 331. In some embodiments, a suitable poloxamer is poloxamer 333. In some embodiments, a suitable poloxamer is poloxamer 334. In some embodiments, a suitable poloxamer is poloxamer 335. In some embodiments, a suitable poloxamer is poloxamer 338. In some embodiments, a suitable poloxamer is poloxamer 401. In some embodiments, a suitable poloxamer is poloxamer 402. In some embodiments, a suitable poloxamer is poloxamer 403. In some embodiments, a suitable poloxamer is poloxamer 407. In some embodiments, a suitable poloxamer is a combination thereof.

In some embodiments, a suitable poloxamer has an average molecular weight of about 4,000 g/mol to about 20,000 g/mol. In some embodiments, a suitable poloxamer has an average molecular weight of about 1,000 g/mol to about 50,000 g/mol. In some embodiments, a suitable poloxamer has an average molecular weight of about 1,000 g/mol. In some embodiments, a suitable poloxamer has an average molecular weight of about 2,000 g/mol. In some embodiments, a suitable poloxamer has an average molecular weight of about 3,000 g/mol. In some embodiments, a suitable poloxamer has an average molecular weight of about 4,000 g/mol. In some embodiments, a suitable poloxamer has an average molecular weight of about 5,000 g/mol. In some embodiments, a suitable poloxamer has an average molecular weight of about 6,000 g/mol. In some embodiments, a suitable poloxamer has an average molecular weight of about 7,000 g/mol. In some embodiments, a suitable poloxamer has an average molecular weight of about 8,000 g/mol. In some embodiments, a suitable poloxamer has an average molecular weight of about 9,000 g/mol. In some embodiments, a suitable poloxamer has an average molecular weight of about 10,000 g/mol. In some embodiments, a suitable poloxamer has an average molecular weight of about 20,000 g/mol. In some embodiments, a suitable poloxamer has an average molecular weight of about 25,000 g/mol. In some embodiments, a suitable poloxamer has an average molecular weight of about 30,000 g/mol. In some embodiments, a suitable poloxamer has an average molecular weight of about 40,000 g/mol. In some embodiments, a suitable poloxamer has an average molecular weight of about 50,000 g/mol.

### Other Amphiphilic Polymers

In some embodiments, an amphiphilic polymer is a poloxamine, e.g., tetronic 304 or tetronic 904.

In some embodiments, an amphiphilic polymer is a polyvinylpyrrolidone (PVP), such as PVP with molecular weight of 3 kDa, 10 kDa, or 29 kDa.

In some embodiments, an amphiphilic polymer is a polyethylene glycol ether (Brij), polysorbate, sorbitan, and derivatives thereof. In some embodiments, an amphiphilic polymer is a polysorbate, such as PS 20.

In some embodiments, an amphiphilic polymer is polyethylene glycol ether (Brij), poloxamer, polysorbate, sorbitan, or derivatives thereof.

In some embodiments, an amphiphilic polymer is a polyethylene glycol ether. In some embodiments, a suitable polyethylene glycol ether is a compound of Formula (S-1):

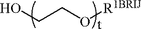

or a salt or isomer thereof, wherein:
t is an integer between 1 and 100;
R^{1BRIJ}independently is C₁₀₋₄₀alkyl, C₁₀₋₄₀alkenyl, or C₁₀₋₄₀alkynyl; and optionally one or more methylene groups of R^{5PEG}are independently replaced with C₃₋₁₀ carbocyclylene, 4 to 10 membered heterocyclylene, C₆₋₁₀arylene, 4 to 10 membered heteroarylene, —N(R^{N})—, —O—, —S—, —C(O)—, —C(O)N(R^{N})—, —NR^{N}C(O)—, —NR C(O)N(R )—, —C(O)O— —OC(O)—, —OC(O)O— — OC(O)N(R^{N})—, —NR^{N}C(O)O— —C(O)S— —SC(O)—, —C(═NR^{N})—,—C(═NR )N(R )—, — NRNC(═NR^{N})— —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O— —OS(O)O— —OS(O)₂— —S(O)₂O— —OS(O)₂O— —N(R^{N})S(O)—, — S(O)N(R^{N})— —N(R^{N})S(O)N(R^{N})— —OS(O)N(R^{N})— —N(R^{N})S(O)0— —S(O)₂— —N(R^{N})S(O)₂— — S(O)₂N(R^{N})—, —N(R^{N})S(O)₂N(R^{N})— —OS(O)₂N(R^{N})— or —N(R^{N})S(O)₂O—; and
each instance of R^{N}is independently hydrogen, C₁₋₆alkyl, or a nitrogen protecting group.

In some embodiment, R^{1BRIJ}is C is alkyl. For example, the polyethylene glycol ether is a compound of Formula (S-1a):

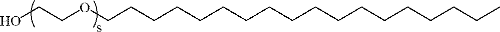

or a salt or isomer thereof, wherein s is an integer between 1 and 100.

In some embodiments, R^{1BRIJ}is C is alkenyl. For example, a suitable polyethylene glycol ether is a compound of Formula (S-1b):

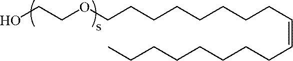

or a salt or isomer thereof, wherein s is an integer between 1 and 100.

Typically, an amphiphilic polymer (e.g., a poloxamer) is present in a formulation at an amount lower than its critical micelle concentration (CMC). In some embodiments, an amphiphilic polymer (e.g., a poloxamer) is present in the mixture at an amount about 1%, about 2%, about 3%, about 4%, about 5%, about 6%, about 7%, about 8%, about 9%, about 10%, about 15%, about 20%, about 25%, about 30%, about 35%, about 40%, about 45%, or about 50% lower than its CMC. In some embodiments, an amphiphilic polymer (e.g., a poloxamer) is present in the mixture at an amount about 0.9%, 0.8%, 0.7%, 0.6%, 0.5%, 0.4%, 0.3%, 0.2%, 0.1% lower than its CMC. In some embodiments, an amphiphilic polymer (e.g., a poloxamer) is present in the mixture at an amount about 55%, 60%, 65%, 70%, 75%, 80%, 90%, or 95% lower than its CMC.

In some embodiments, less than about 0.1%, 0.09%, 0.08%, 0.07%, 0.06%, 0.05%, 0.04%, 0.03%, 0.02%, or 0.01% of the original amount of the amphiphilic polymer (e.g., the poloxamer) present in the formulation remains upon removal. In some embodiments, a residual amount of the amphiphilic polymer (e.g., the poloxamer) remains in a formulation upon removal. As used herein, a residual amount means a remaining amount after substantially all of the substance (an amphiphilic polymer described herein such as a poloxamer) in a composition is removed. A residual amount may be detectable using a known technique qualitatively or quantitatively. A residual amount may not be detectable using a known technique.

In some embodiments, a suitable delivery vehicle comprises less than 5% amphiphilic block copolymers (e.g., poloxamers). In some embodiments, a suitable delivery vehicle comprises less than 3% amphiphilic block copolymers (e.g., poloxamers). In some embodiments, a suitable delivery vehicle comprises less than 2.5% amphiphilic block copolymers (e.g., poloxamers). In some embodiments, suitable delivery vehicle comprises less than 2% amphiphilic block copolymers (e.g., poloxamers). In some embodiments, a suitable delivery vehicle comprises less than 1.5% amphiphilic block copolymers (e.g., poloxamers). In some embodiments, a suitable delivery vehicle comprises less than 1% amphiphilic block copolymers (e.g., poloxamers). In some embodiments, a suitable delivery vehicle comprises less than 0.5% (e.g., less than 0.4%, 0.3%, 0.2%, 0.1%) amphiphilic block copolymers (e.g., poloxamers). In some embodiments, a suitable delivery vehicle comprises less than 0.09%, 0.08%, 0.07%, 0.06%, 0.05%, 0.04%, 0.03%, 0.02%, or 0.01% amphiphilic block copolymers (e.g., poloxamers). In some embodiments, a suitable delivery vehicle comprises less than 0.01% amphiphilic block copolymers (e.g., poloxamers). In some embodiments, a suitable delivery vehicle contains a residual amount of amphiphilic polymers (e.g., poloxamers). As used herein, a residual amount means a remaining amount after substantially all of the substance (an amphiphilic polymer described herein such as a poloxamer) in a composition is removed. A residual amount may be detectable using a known technique qualitatively or quantitatively. A residual amount may not be detectable using a known technique.

### Polymers

In some embodiments, a suitable delivery vehicle is formulated using a polymer as a carrier, alone or in combination with other carriers including various lipids described herein. Thus, in some embodiments, liposomal delivery vehicles, as used herein, also encompass nanoparticles comprising polymers. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDa, e.g., 25 kDa branched PEI (Sigma #408727).

According to various embodiments, the selection of cationic lipids, non-cationic lipids, PEG-modified lipids, cholesterol-based lipids, and/or amphiphilic block copolymers which comprise the lipid nanoparticle, as well as the relative molar ratio of such components (lipids) to each other, is based upon the characteristics of the selected lipid(s), the nature of the intended target cells, the characteristics of the nucleic acid to be delivered. Additional considerations include, for example, the saturation of the alkyl chain, as well as the size, charge, pH, pKa, fusogenicity and toxicity of the selected lipid(s). Thus the molar ratios may be adjusted accordingly.

### Liposomes Suitable for Use With the Present Invention

A suitable liposome for the present invention may include one or more of any of the cationic lipids, non-cationic lipids, cholesterol lipids, PEG-modified lipids, amphiphilic block copolymers and/or polymers described herein at various ratios. In some embodiments, a lipid nanoparticle comprises five and no more than five distinct components of nanoparticle. In some embodiments, a lipid nanoparticle comprises four and no more than four distinct components of nanoparticle. In some embodiments, a lipid nanoparticle comprises three and no more than three distinct components of nanoparticle. As non-limiting examples, a suitable liposome formulation may include a combination selected from cKK-E 12, DOPE, cholesterol and DMG-PEG2K; C 12-200, DOPE, cholesterol and DMG-PEG2K; HGT4003, DOPE, cholesterol and DMG-PEG2K; ICE, DOPE, cholesterol and DMG-PEG2K; or ICE, DOPE, and DMG-PEG2K.

In particular embodiments, a liposome for use with this invention comprises a lipid component consisting of a cationic lipid, a non-cationic lipid (e.g., DOPE or DEPE), a PEG-modified lipid (e.g., DMG-PEG2K), and optionally cholesterol. Cationic lipids particularly suitable for inclusion in such a liposome include GL-TES-SA-DME-E18-2, TL1-01D-DMA, SY-3-E14-DMAPr, TL1-10D-DMA, HGT4002 (also referred to herein as Guan-SS-Chol), GL-TES-SA-DMP-E18-2, HEP-E4-E10, HEP-E3-E10, and TL1-04D-DMA. These cationic lipids have been found to be particularly suitable for use in liposomes that are administered through pulmonary delivery via nebulization. Amongst these, HEP-E4-E10, HEP-E3-E10, GL-TES-SA-DME-E18-2, GL-TES-SA-DMP-E18-2, TL1-01D-DMA and TL1-04D-DMA performed particularly well.

In some embodiments, an HBEC-ALI (Human Bronchial Epithelial Cell - Air Liquid Interface) system can be used to assess mucociliary transport (MCT) using micro-optical coherence tomography (uOCT), e.g., by visualizing MCT using fluorescent microbeads. Accordingly, HBEC-ALI can be used to assess the potency of a liposome encapsulating a codon-optimized DNAI1 mRNA sequence for use in treating primary ciliary dyskinesia (PCD).

Exemplary liposomes for use with the present invention include one of GL-TES-SA-DME-E18-2, TL1-01D-DMA, SY-3-E14-DMAPr, TL1-10D-DMA, GL-TES-SA-DMP-E18-2, HEP-E4-E10, HEP-E3-E10 and TL1-04D-DMA as a cationic lipid component, DOPE as a non-cationic lipid component, cholesterol as a helper lipid component, and DMG-PEG2K as a PEG-modified lipid component. In some embodiments, the molar ratio of the cationic lipid to non-cationic lipid to cholesterol to PEG-modified lipid may be between about 30-60:25-35:20-30:1-15, respectively. In some embodiments, the molar ratio of cationic lipid to non-cationic lipid to cholesterol to PEG-modified lipid is approximately 40:30:20:10, respectively. In some embodiments, the molar ratio of cationic lipid to non-cationic lipid to cholesterol to PEG-modified lipid is approximately 40:30:25:5, respectively. In some embodiments, the molar ratio of cationic lipid to non-cationic lipid to cholesterol to PEG-modified lipid is approximately 40:32:25:3, respectively. In some embodiments, the molar ratio of cationic lipid to non-cationic lipid to cholesterol to PEG-modified lipid is approximately 50:25:20:5.

In some embodiments, the lipid component of a liposome particularly suitable for pulmonary delivery consists of HGT4002 (also referred to herein as Guan-SS-Chol), DOPE and DMG-PEG2K. In some embodiments, the molar ratio of cationic lipid to non-cationic lipid to PEG-modified lipid is approximately 60:35:5.

In various embodiments, cationic lipids (e.g., cKK-E12, C12-200, ICE, and/or HGT4003) constitute about 30-60 % (e.g., about 30-55%, about 30-50%, about 30-45%, about 30-40%, about 35-50%, about 35-45%, or about 35-40%) of the liposome by molar ratio. In some embodiments, the percentage of cationic lipids (e.g., cKK-E12, C12-200, ICE, and/or HGT4003) is or greater than about 30%, about 35%, about 40 %, about 45%, about 50%, about 55%, or about 60% of the liposome by molar ratio.

In some embodiments, the ratio of cationic lipid(s) to non-cationic lipid(s) to cholesterol-based lipid(s) to PEG-modified lipid(s) may be between about 30-60:25-35:20-30:1-15, respectively. In some embodiments, the ratio of cationic lipid(s) to non-cationic lipid(s) to cholesterol-based lipid(s) to PEG-modified lipid(s) is approximately 40:30:20:10, respectively. In some embodiments, the ratio of cationic lipid(s) to non-cationic lipid(s) to cholesterol-based lipid(s) to PEG-modified lipid(s) is approximately 40:30:25:5, respectively. In some embodiments, the ratio of cationic lipid(s) to non-cationic lipid(s) to cholesterol-based lipid(s) to PEG-modified lipid(s) is approximately 40:32:25:3, respectively. In some embodiments, the ratio of cationic lipid(s) to non-cationic lipid(s) to cholesterol-based lipid(s) to PEG-modified lipid(s) is approximately 50:25:20:5.

### Ratio of Distinct Lipid Components

In embodiments where a lipid nanoparticle comprises three and no more than three distinct components of lipids, the ratio of total lipid content (i.e., the ratio of lipid component (1):lipid component (2):lipid component (3)) can be represented as x:y:z, wherein

$$\left(\text{y} + \text{z}\right) = 100 - \text{x} .$$

In some embodiments, each of “x,” “y,” and “z” represents molar percentages of the three distinct components of lipids, and the ratio is a molar ratio.

In some embodiments, each of “x,” “y,” and “z” represents weight percentages of the three distinct components of lipids, and the ratio is a weight ratio.

In some embodiments, lipid component (1), represented by variable “x,” is a sterol-based cationic lipid.

In some embodiments, lipid component (2), represented by variable “y,” is a helper lipid.

In some embodiments, lipid component (3), represented by variable “z” is a PEG lipid.

In some embodiments, variable “x,” representing the molar percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the molar percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the molar percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “x,” representing the weight percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the weight percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the weight percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “z,” representing the molar percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the molar percentage of lipid component (3) (e.g., a PEG lipid) is about 1%, 2%, 3%, 4%, 5%, 6%, 7%, 8%, 9%, 10%. In embodiments, variable “z,” representing the molar percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

In some embodiments, variable “z,” representing the weight percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the weight percentage of lipid component (3) (e.g., a PEG lipid) is about 1%, 2%, 3%, 4%, 5%, 6%, 7%, 8%, 9%, 10%. In embodiments, variable “z,” representing the weight percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

For compositions having three and only three distinct lipid components, variables “x,” “y,” and “z” may be in any combination so long as the total of the three variables sums to 100% of the total lipid content.

### Formation of Liposomes Encapsulating mRNA

The liposomal transfer vehicles for use in the compositions of the invention can be prepared by various techniques which are presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion which results in the formation of MLVs. Unilamellar vesicles (ULV) can then be formed by homogenization, sonication or extrusion of the multilamellar vesicles. In addition, unilamellar vesicles can be formed by detergent removal techniques.

Various methods are described in published U.S. Application No. US 2011/0244026, published U.S. Application No. US 2016/0038432 , published U.S. Application No. US 2018/0153822, published U.S. Application No. US 2018/0125989 and U.S. Provisional Application No. 62/877,597, filed Jul. 23, 2019 and can be used to practice the present invention, all of which are incorporated herein by reference. As used herein, Process A refers to a conventional method of encapsulating mRNA by mixing mRNA with a mixture of lipids, without first pre-forming the lipids into lipid nanoparticles, as described in US 2016/0038432. As used herein, Process B refers to a process of encapsulating messenger RNA (mRNA) by mixing pre-formed lipid nanoparticles with mRNA, as described in US 2018/0153822.

Briefly, the process of preparing mRNA- or MCNA-loaded lipid liposomes includes a step of heating one or more of the solutions (i.e., applying heat from a heat source to the solution) to a temperature (or to maintain at a temperature) greater than ambient temperature, the one more solutions being the solution comprising the pre-formed lipid nanoparticles, the solution comprising the mRNA and the mixed solution comprising the lipid nanoparticle encapsulated mRNA. In some embodiments, the process includes the step of heating one or both of the mRNA solution and the pre-formed lipid nanoparticle solution, prior to the mixing step. In some embodiments, the process includes heating one or more one or more of the solution comprising the pre-formed lipid nanoparticles, the solution comprising the mRNA and the solution comprising the lipid nanoparticle encapsulated mRNA, during the mixing step. In some embodiments, the process includes the step of heating the lipid nanoparticle encapsulated mRNA, after the mixing step. In some embodiments, the temperature to which one or more of the solutions is heated (or at which one or more of the solutions is maintained) is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature greater than ambient temperature to which one or more of the solutions is heated is about 65° C.

Various methods may be used to prepare an mRNA solution suitable for the present invention. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps.

Typically, the buffer solution is mixed at a rate greater than that of the mRNA stock solution. For example, the buffer solution may be mixed at a rate at least 1x, 2x, 3x, 4x, 5x, 6x, 7x, 8x, 9x, 10x, 15x, or 20x greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of or greater than about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

According to the present invention, a lipid solution contains a mixture of lipids suitable to form lipid nanoparticles for encapsulation of mRNA. In some embodiments, a suitable lipid solution is ethanol based. For example, a suitable lipid solution may contain a mixture of desired lipids dissolved in pure ethanol (i.e., 100% ethanol). In another embodiment, a suitable lipid solution is isopropyl alcohol based. In another embodiment, a suitable lipid solution is dimethylsulfoxide-based. In another embodiment, a suitable lipid solution is a mixture of suitable solvents including, but not limited to, ethanol, isopropyl alcohol and dimethylsulfoxide.

A suitable lipid solution may contain a mixture of desired lipids at various concentrations. For example, a suitable lipid solution may contain a mixture of desired lipids at a total concentration of or greater than about 0.1 mg/ml, 0.5 mg/ml, 1.0 mg/ml, 2.0 mg/ml, 3.0 mg/ml, 4.0 mg/ml, 5.0 mg/ml, 6.0 mg/ml, 7.0 mg/ml, 8.0 mg/ml, 9.0 mg/ml, 10 mg/ml, 15 mg/ml, 20 mg/ml, 30 mg/ml, 40 mg/ml, 50 mg/ml, or 100 mg/ml. In some embodiments, a suitable lipid solution may contain a mixture of desired lipids at a total concentration ranging from about 0.1-100 mg/ml, 0.5-90 mg/ml, 1.0-80 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml. In some embodiments, a suitable lipid solution may contain a mixture of desired lipids at a total concentration up to about 100 mg/ml, 90 mg/ml, 80 mg/ml, 70 mg/ml, 60 mg/ml, 50 mg/ml, 40 mg/ml, 30 mg/ml, 20 mg/ml, or 10 mg/ml.

Any desired lipids may be mixed at any ratios suitable for encapsulating mRNAs. In some embodiments, a suitable lipid solution contains a mixture of desired lipids including cationic lipids, helper lipids (e.g. non cationic lipids and/or cholesterol lipids), amphiphilic block copolymers (e.g. poloxamers) and/or PEGylated lipids. In some embodiments, a suitable lipid solution contains a mixture of desired lipids including one or more cationic lipids, one or more helper lipids (e.g. non cationic lipids and/or cholesterol lipids) and one or more PEGylated lipids.

In certain embodiments, provided compositions comprise a liposome wherein the mRNA is associated on both the surface of the liposome and encapsulated within the same liposome. For example, during preparation of the compositions of the present invention, cationic liposomes may associate with the mRNA or MCNA through electrostatic interactions.

In some embodiments, the compositions and methods of the invention comprise mRNA encapsulated in a liposome. In some embodiments, the one or more mRNA species may be encapsulated in the same liposome. In some embodiments, the one or more mRNA species may be encapsulated in different liposomes. In some embodiments, the mRNA is encapsulated in one or more liposomes, which differ in their lipid composition, molar ratio of lipid components, size, charge (zeta potential), targeting ligands and/or combinations thereof. In some embodiments, the one or more liposome may have a different composition of sterol-based cationic lipids, neutral lipid, PEG-modified lipid and/or combinations thereof. In some embodiments the one or more liposomes may have a different molar ratio of cholesterol-based cationic lipid, neutral lipid, and PEG-modified lipid used to create the liposome.

The process of incorporation of a desired nucleic acid (e.g., mRNA or MCNA) into a liposome is often referred to as “loading”. Exemplary methods are described in Lasic, et al. FEBS Lett., 312: 255-258, 1992, which is incorporated herein by reference. The liposome-incorporated nucleic acids may be completely or partially located in the interior space of the liposome, within the bilayer membrane of the liposome, or associated with the exterior surface of the liposome membrane. The incorporation of a nucleic acid into liposomes is also referred to herein as “encapsulation” wherein the nucleic acid is entirely contained within the interior space of the liposome. The purpose of incorporating an mRNA into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in some embodiments, a suitable delivery vehicle is capable of enhancing the stability of the mRNA contained therein and/or facilitate the delivery of therapeutic agent (e.g., mRNA or MCNA) to the target cell or tissue.

Suitable liposomes in accordance with the present invention may be made in various sizes. In some embodiments, provided liposomes may be made smaller than previously known liposomes. In some embodiments, decreased size of liposomes is associated with more efficient delivery of therapeutic agent (e.g., mRNA or MCNA). Selection of an appropriate liposome size may take into consideration the site of the target cell or tissue and to some extent the application for which the liposome is being made.

In some embodiments, an appropriate size of liposome is selected to facilitate systemic distribution of antibody encoded by the mRNA. In some embodiments, it may be desirable to limit transfection of the mRNA to certain cells or tissues. For example, to target hepatocytes a liposome may be sized such that its dimensions are smaller than the fenestrations of the endothelial layer lining hepatic sinusoids in the liver; in such cases the liposome could readily penetrate such endothelial fenestrations to reach the target hepatocytes.

Alternatively or additionally, a liposome may be sized such that the dimensions of the liposome are of a sufficient diameter to limit or expressly avoid distribution into certain cells or tissues.

A variety of alternative methods known in the art are available for sizing of a population of liposomes. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomes may be determined by quasi-electric light scattering (QELS) as described in Bloomfield, Ann. Rev. Biophys. Bioeng., 10:421-450 (1981), incorporated herein by reference. Average liposome diameter may be reduced by sonication of formed liposomes. Intermittent sonication cycles may be alternated with QELS assessment to guide efficient liposome synthesis.

### Provided Nanoparticles Encapsulating mRNA

In some embodiments, majority of purified nanoparticles in a composition, i.e., greater than about 50%, 55%, 60%, 65%, 70%, 75%, 80%, 85%, 90%, 95%, 96%, 97%, 98%, or 99% of the nanoparticles, have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, about 80 nm, about 75 nm, about 70 nm, about 65 nm, about 60 nm, about 55 nm, about 50 nm, about 45 nm, about 40 nm, about 35 nm, or about 30 nm). In some embodiments, substantially all of the purified nanoparticles have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, about 80 nm, about 75 nm, about 70 nm, about 65 nm, about 60 nm, about 55 nm, about 50 nm, about 45 nm, about 40 nm, about 35 nm, or about 30 nm).

In some embodiments, a lipid nanoparticle has an average size of less than 150 nm. In some embodiments, a lipid nanoparticle has an average size of less than 120 nm. In some embodiments, a lipid nanoparticle has an average size of less than 100 nm. In some embodiments, a lipid nanoparticle has an average size of less than 90 nm. In some embodiments, a lipid nanoparticle has an average size of less than 80 nm. In some embodiments, a lipid nanoparticle has an average size of less than 70 nm. In some embodiments, a lipid nanoparticle has an average size of less than 60 nm. In some embodiments, a lipid nanoparticle has an average size of less than 50 nm. In some embodiments, a lipid nanoparticle has an average size of less than 30 nm. In some embodiments, a lipid nanoparticle has an average size of less than 20 nm.

In some embodiments, the dispersity, or measure of heterogeneity in size of molecules (PDI), of nanoparticles in a composition provided by the present invention is less than about 0.5. In some embodiments, a lipid nanoparticle has a PDI of less than about 0.5. In some embodiments, a lipid nanoparticle has a PDI of less than about 0.4. In some embodiments, a lipid nanoparticle has a PDI of less than about 0.3. In some embodiments, a lipid nanoparticle has a PDI of less than about 0.28. In some embodiments, a lipid nanoparticle has a PDI of less than about 0.25. In some embodiments, a lipid nanoparticle has a PDI of less than about 0.23. In some embodiments, a lipid nanoparticle has a PDI of less than about 0.20. In some embodiments, a lipid nanoparticle has a PDI of less than about 0.18. In some embodiments, a lipid nanoparticle has a PDI of less than about 0.16. In some embodiments, a lipid nanoparticle has a PDI of less than about 0.14. In some embodiments, a lipid nanoparticle has a PDI of less than about 0.12. In some embodiments, a lipid nanoparticle has a PDI of less than about 0.10. In some embodiments, a lipid nanoparticle has a PDI of less than about 0.08.

In some embodiments, greater than about 75%, 80%, 85%, 90%, 95%, 96%, 97%, 98%, or 99% of the purified lipid nanoparticles in a composition provided by the present invention encapsulate an mRNA within each individual particle. In some embodiments, substantially all of the purified lipid nanoparticles in a composition encapsulate an mRNA within each individual particle. In some embodiments, a lipid nanoparticle has an encapsulation efficiency of between 50% and 99%. In some embodiments, a lipid nanoparticle has an encapsulation efficiency of greater than about 60%. In some embodiments, a lipid nanoparticle has an encapsulation efficiency of greater than about 65%. In some embodiments, a lipid nanoparticle has an encapsulation efficiency of greater than about 70%. In some embodiments, a lipid nanoparticle has an encapsulation efficiency of greater than about 75%. In some embodiments, a lipid nanoparticle has an encapsulation efficiency of greater than about 80%. In some embodiments, a lipid nanoparticle has an encapsulation efficiency of greater than about 85%. In some embodiments, a lipid nanoparticle has an encapsulation efficiency of greater than about 90%. In some embodiments, a lipid nanoparticle has an encapsulation efficiency of greater than about 92%. In some embodiments, a lipid nanoparticle has an encapsulation efficiency of greater than about 95%. In some embodiments, a lipid nanoparticle has an encapsulation efficiency of greater than about 98%. In some embodiments, a lipid nanoparticle has an encapsulation efficiency of greater than about 99%.

In some embodiments, a lipid nanoparticle has a N/P ratio of between 1 and 10. As used herein, the term “N/P ratio” refers to a molar ratio of positively charged molecular units in the cationic lipids in a lipid nanoparticle relative to negatively charged molecular units in the mRNA encapsulated within that lipid nanoparticle. As such, N/P ratio is typically calculated as the ratio of moles of amine groups in cationic lipids in a lipid nanoparticle relative to moles of phosphate groups in mRNA encapsulated within that lipid nanoparticle. In some embodiments, a lipid nanoparticle has a N/P ratio above 1. In some embodiments, a lipid nanoparticle has a N/P ratio of about 1. In some embodiments, a lipid nanoparticle has a N/P ratio of about 2. In some embodiments, a lipid nanoparticle has a N/P ratio of about 3. In some embodiments, a lipid nanoparticle has a N/P ratio of about 4. In some embodiments, a lipid nanoparticle has a N/P ratio of about 5. In some embodiments, a lipid nanoparticle has a N/P ratio of about 6. In some embodiments, a lipid nanoparticle has a N/P ratio of about 7. In some embodiments, a lipid nanoparticle has a N/P ratio of about 8.

In some embodiments, a composition according to the present invention contains at least about 0.5 mg, 1 mg, 5 mg, 10 mg, 100 mg, 500 mg, or 1000 mg of encapsulated mRNA. In some embodiments, a composition contains about 0.1 mg to 1000 mg of encapsulated mRNA. In some embodiments, a composition contains at least about 0.5 mg of encapsulated mRNA. In some embodiments, a composition contains at least about 0.8 mg of encapsulated mRNA. In some embodiments, a composition contains at least about 1 mg of encapsulated mRNA. In some embodiments, a composition contains at least about 5 mg of encapsulated mRNA. In some embodiments, a composition contains at least about 8 mg of encapsulated mRNA. In some embodiments, a composition contains at least about 10 mg of encapsulated mRNA. In some embodiments, a composition contains at least about 50 mg of encapsulated mRNA. In some embodiments, a composition contains at least about 100 mg of encapsulated mRNA. In some embodiments, a composition contains at least about 500 mg of encapsulated mRNA. In some embodiments, a composition contains at least about 1000 mg of encapsulated mRNA.

### Pharmaceutical Compositions

The present invention provides compositions for use in the treatment of primary ciliary dyskinesia (PCD). The compositions of the present invention are for use in the manufacture of a medicament for the treatment of primary ciliary dyskinesia (PCD).

Provided liposomally-encapsulated or associated mRNAs, and compositions containing the same, may be administered and dosed in accordance with current medical practice, taking into account the clinical condition of the subject, the site and method of administration, the scheduling of administration, the subject’s age, sex, body weight and other factors relevant to clinicians of ordinary skill in the art. As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject, the mammal, (e.g., treating, modulating, curing, preventing and/or ameliorating PCD). For example, a therapeutically effective amount may be an amount sufficient to achieve a desired therapeutic and/or prophylactic effect. Generally, the amount of a therapeutic agent (e.g., mRNA encoding a DNAI1 protein) administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays may optionally be employed to identify optimal dosage ranges.

In some embodiments, an effective therapeutic dose of the pharmaceutical composition comprising an mRNA encoding dynein axonemal intermediate chain 1 protein is administered to the mammal at a dosing interval sufficient to reduce for the period of the dosing interval or longer the level of at least one symptom or biomarker associated with PCD in the mammal relative to its state prior to the treatment.

In some embodiments the mammal is a human. A suitable therapeutic dose that may be applicable for a human being can be derived based on animal studies. A basic guideline for deriving a human equivalent dose from studies performed in animals can be obtained from the U.S> Food and Drug Administration (FDA) website at https://www.fda.gov/downloads/drugs/guidances/ucm078932.pdf, entitled, “Guidance for Industry Estimating the Maximum Safe Starting Dose in Initial Clinical Trials for Therapeutics in Adult Healthy Volunteers.” Based on the guidelines for allometric scaling, a suitable dose of, for example, 0.6 mg/kg in a mouse, would relate to a human equivalent dose of 0.0048 mg/kg. Thus, considering the derived human equivalent dose, a projected human therapeutic dose can be derived based on studies in other animals.

In some embodiments, the dosing interval is once every 15 days or longer, or once every 20 days or longer, or once every 21 days, or once every 22 days, or once every 23 days, or once every 24 days, or once every 25 days, once every 26 days, or once every 27 days, or once every 28 days, or once every 29 days or longer, or once every 30 days or longer, or once every 31 days or longer. In some embodiments, the dosing interval is once every 40, 45 or 50 days or 60 days, or any number of days in between. In some embodiments, the dosing interval is once every 80, 90 or 120 days or 150 days, or any number of days in between.

In some embodiments, the therapeutic low dose is administered at a dosing interval of once every 2 weeks or longer, which is sufficient to reduce the level of at least one symptom or biomarker associated with PCD in the mammal relative to the state prior to the treatment. In some embodiments, the therapeutic low dose is administered at a dosing interval of once every 3 weeks or longer, which is sufficient to reduce the level of at least one symptom or biomarker associated with PCD in the mammal relative to the state prior to the treatment. In some embodiments, the dosing interval is once every 4 weeks or longer. In some embodiments, the dosing interval is once every 5 weeks or longer. In some embodiments, the dosing interval is once every 6 weeks or longer. In some embodiments, the dosing interval is once every 8 weeks or longer. In some embodiments, the dosing interval is once every 12 or 15 or 18 weeks or longer.

In some embodiments, the dosing interval is once a month. In some embodiments, the dosing interval is once in every two months. In some embodiments, the dosing interval is once every three months, or once every four months or once every five months or once every six months or anywhere in between.

In some embodiments, administering the provided composition results in an increased DNAI1 mRNA expression level in a biological sample from a subject as compared to a baseline expression level before treatment. Typically, the baseline level is measured immediately before treatment. Biological samples include, for example, whole blood, serum, plasma, urine and tissue samples (e.g., muscle, liver, skin fibroblasts). In some embodiments, administering the provided composition results in an increased DNAI1 mRNA expression level by at least about 10%, 20%, 30%, 40%, 50%, 60%, 70%, 80%, 90%, or 95% as compared to the baseline level immediately before treatment. In some embodiments, administering the provided composition results in an increased DNAI1 mRNA expression level as compared to a DNAI1 mRNA expression level in subjects who are not treated

According to the present invention, a therapeutically effective dose of the provided composition, when administered regularly, results in an increased DNAI1 protein expression or activity level in a subject as compared to a baseline DNAI1 protein expression or activity level before treatment. Typically, the DNAI1 protein expression or activity level is measured in a biological sample obtained from the subject such as blood, plasma or serum, urine, or solid tissue extracts. In some embodiments, the administering of a composition of the invention results in DNAI1 expression detectable in the liver. In some embodiments, administering the provided composition results in an increased DNAI1 protein expression or activity level in a biological sample (e.g., plasma/serum or urine) by at least about 10%, 20%, 30%, 40%, 50%, 60%, 70%, 80%, 90%, or 95% as compared to a baseline level before treatment. In some embodiments, administering the provided composition results in an increased DNAI1 protein expression or activity level in a biological sample (e.g., plasma/serum or urine) by at least about 10%, 20%, 30%, 40%, 50%, 60%, 70%, 80%, 90%, or 95% as compared to a baseline level before treatment for at least 24 hours, at least 48 hours, at least 72 hours, at least 3 days, at least 4 days, at least 5 days, at least 6 days, at least 7 days, at least 8 days, at least 9 days, at least 10 days, at least 11 days, at least 12 days, at least 13 days, at least 14 days, or at least 15 days.

In some embodiments, the therapeutic dose is sufficient to achieve at least some stabilization, improvement or elimination of symptoms and other indicators, such as biomarkers, are selected as appropriate measures of disease progress, disease regression or improvement by those of skill in the art.

Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal.

In some embodiments, the therapeutically effective dose comprising the mRNA encoding DNAI1 protein is administered to the subject by intramuscular administration.

In some embodiments, the therapeutically effective dose comprising the mRNA encoding DNAI1 is administered to the subject by subcutaneous administration.

In particular embodiments, the intramuscular administration is to a muscle selected from the group consisting of skeletal muscle, smooth muscle and cardiac muscle. In some embodiments the administration results in delivery of the mRNA to a muscle cell. In some embodiments the administration results in delivery of the mRNA to a hepatocyte (i.e., liver cell). In a particular embodiment, the intramuscular administration results in delivery of the mRNA to a muscle cell.

Most commonly, the therapeutically effective dose comprising the mRNA encoding dynein axonemal intermediate chain protein 1 is administered to the subject by intravenous administration.

Alternatively or additionally, liposomally encapsulated mRNAs and compositions of the invention may be administered in a local rather than systemic manner, for example, via injection of the pharmaceutical composition directly into a targeted tissue, preferably in a sustained release formulation. Local delivery can be affected in various ways, depending on the tissue to be targeted. For example, aerosols containing compositions of the present invention can be inhaled (for nasal, tracheal, or bronchial delivery); compositions of the present invention can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing provided compositions complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

In particular embodiments, DNAI1 encoding mRNA is administered intravenously, wherein intravenous administration is associated with delivery of the mRNA to hepatocytes.

In some embodiments, the therapeutically effective dose comprising the mRNA encoding dynein axonemal intermediate chain protein is administered for suitable delivery to the mammal’s liver. In some embodiments, the therapeutically effective dose comprising the mRNA encoding dynein axonemal intermediate chain protein is administered for suitable expression in hepatocytes of the administered mammal.

Provided methods of the present invention contemplate single as well as multiple administrations of a therapeutically effective amount of the therapeutic agents (e.g., mRNA encoding a DNAI1 protein) described herein. Therapeutic agents can be administered at regular intervals, depending on the nature, severity and extent of the subject’s condition (e.g., PCD). In some embodiments, a therapeutically effective amount of the therapeutic agents (e.g., mRNA encoding a DNAI1 protein) of the present invention may be administered intrathecally periodically at regular intervals (e.g., once every year, once every six months, once every five months, once every three months, bimonthly (once every two months), monthly (once every month), biweekly (once every two weeks), twice a month, once every 30 days, once every 28 days, once every 14 days, once every 10 days, once every 7 days, weekly, twice a week, daily or continuously).

In some embodiments, provided liposomes and/or compositions are formulated such that they are suitable for extended-release of the mRNA contained therein. Such extended-release compositions may be conveniently administered to a subject at extended dosing intervals. For example, in one embodiment, the compositions of the present invention are administered to a subject twice a day, daily or every other day. In some embodiments, the compositions of the present invention are administered to a subject twice a week, once a week, once every 7 days, once every 10 days, once every 14 days, once every 28 days, once every 30 days, once every two weeks, once every three weeks, once every four weeks, once a month, twice a month, once every six weeks, once every eight weeks, once every other month, once every three months, once every four months, once every six months, once every eight months, once every nine months or annually.

In a preferred embodiment, the compositions of the present invention are administered to a subject once a week, once every two weeks or once a month. In a more preferred embodiment, the compositions of the present invention are administered to a subject once every two weeks or once every month. In the most preferred embodiment, the compositions of the present invention are administered to a subject once every month.

In some embodiments the mRNA is administered concurrently with an additional therapy.

Also contemplated are compositions and liposomes which are formulated for depot administration (e.g., intramuscularly, subcutaneously, intravitreally) to either deliver or release an mRNA over extended periods of time. Preferably, the extended-release means employed are combined with modifications made to the mRNA to enhance stability.

A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific protein employed; the duration of the treatment; and like factors as is well known in the medical arts. According to the present invention, a therapeutically effective dose of the provided composition, when administered regularly, results in at least one symptom or feature of PCD is reduced in intensity, severity, or frequency or has delayed onset.

Also contemplated herein are lyophilized pharmaceutical compositions comprising one or more of the liposomes disclosed herein and related methods for the use of such compositions as disclosed for example, in International Patent Application PCT/US 12/41663, filed Jun. 8, 2012, the teachings of which are incorporated herein by reference in their entirety. For example, lyophilized pharmaceutical compositions according to the invention may be reconstituted prior to administration or can be reconstituted in vivo. For example, a lyophilized pharmaceutical composition can be formulated in an appropriate dosage form (e.g., an intradermal dosage form such as a disk, rod or membrane) and administered such that the dosage form is rehydrated over time in vivo by the individual’s bodily fluids.

In some embodiments, the pharmaceutical composition comprises a lyophilized liposomal delivery vehicle that comprises a cationic lipid, a non-cationic lipid, a PEG-modified lipid and cholesterol. In some embodiments, the pharmaceutical composition has a Dv50 of less than 500 nm, 300 nm, 200 nm, 150 nm, 125 nm, 120 nm, 100 nm, 75 nm, 50 nm, 25 nm or smaller upon reconstitution. In some embodiments, the pharmaceutical composition has a Dv90 of less than 750 nm, 700 nm, 500 nm, 300 nm, 200 nm, 150 nm, 125 nm, 100 nm, 75 nm, 50 nm, 25 nm or smaller upon reconstitution. In some embodiments, the pharmaceutical composition has a polydispersity index value of less than 1, 0.95, 0.9, 0.8, 0.75, 0.7, 0.6, 0.5, 0.4, 0.3, 0.25, 0.2, 0.1, 0.05 or less upon reconstitution. In some embodiments, the pharmaceutical composition has an average particle size of less than 500 nm, 400 nm, 300 nm, 200 nm, 175 nm, 150 nm, 125 nm, 100 nm, 75 nm, 50 nm, 25 nm or upon reconstitution.

In some embodiments, the lyophilized pharmaceutical composition further comprises one or more lyoprotectants, such as sucrose, trehalose, dextran or inulin. Typically, the lyoprotectant is sucrose. In some embodiments, the pharmaceutical composition is stable for at least 1 month or at least 6 months upon storage at 4° C., or for at least 6 months upon storage at 25° C. In some embodiments, the biologic activity of the mRNA of the reconstituted lyophilized pharmaceutical composition exceeds 75% of the biological activity observed prior to lyophilization of the composition.

Provided liposomes and compositions may be administered to any desired tissue. In some embodiments, the DNAI1 mRNA delivered by provided liposomes or compositions is expressed in the tissue in which the liposomes and/or compositions were administered. In some embodiments, the mRNA delivered is expressed in a tissue different from the tissue in which the liposomes and/or compositions were administered. Exemplary tissues in which delivered mRNA may be delivered and/or expressed include, but are not limited to the liver, kidney, heart, spleen, serum, brain, skeletal muscle, lymph nodes, skin, and/or cerebrospinal fluid.

According to various embodiments, the timing of expression of delivered mRNAs can be tuned to suit a particular medical need. In some embodiments, the expression of the protein encoded by delivered mRNA is detectable 1, 2, 3, 6, 12, 24, 48, 72, 96 hours, 1 week, 2 weeks, or 1 month after administration of provided liposomes and/or compositions.

In some embodiments, administering the provided composition results in an increased level of DNAI1 protein in a liver cell (e.g., a hepatocyte) of a subject as compared to a baseline level before treatment. Typically, the baseline level is measured immediately before treatment. In some embodiments, administering the provided composition results in an increased DNAI1 protein level in the liver cell by at least about 10%, 20%, 30%, 40%, 50%, 60%, 70%, 80%, 90%, or 95% as compared to a baseline level before treatment. In some embodiments, administering the provided composition results in an increased DNAI1 protein level in a liver cell as compared to the DNAI1 protein level a liver cell of subjects who are not treated.

In some embodiments, administering the provided composition results in an increased DNAI1 protein level in plasma or serum of subject as compared to a baseline level before treatment. Typically, the baseline level is measured immediately before treatment. In some embodiments, administering the provided composition results in an increased DNAI1 protein level in plasma or serum by at least about 10%, 20%, 30%, 40%, 50%, 60%, 70%, 80%, 90%, or 95% as compared to a baseline level before treatment. In some embodiments, administering the provided composition results in an increased DNAI1 protein level in plasma or serum as compared to a DNAI1 protein level in plasma or serum of subjects who are not treated.

In some embodiments, administering the provided composition results in increased DNAI1 enzyme activity in a biological sample from a subject as compared to the baseline level before treatment. Typically, the baseline level is measured immediately before treatment. Biological samples include, for example, whole blood, serum, plasma, urine and tissue samples (e.g., liver). In some embodiments, administering the provided composition results in an increased DNAI1 enzyme activity by at least about 10%, 20%, 30%, 40%, 50%, 60%, 70%, 80%, 90%, or 95% as compared to a baseline level immediately before treatment. In some embodiments, administering the provided composition results in an increased DNAI1 enzyme activity as compared to DNAI1 activity in subjects who are not treated.

In some embodiments the subject is a mammal. In some embodiments, the mammal is an adult. In some embodiments the mammal is an adolescent. In some embodiments the mammal is an infant or a young mammal. In some embodiments, the mammal is a primate. In some embodiments the mammal is a human. In some embodiments the subject is 6 years to 80 years old.

### EXAMPLES

While certain compounds, compositions and methods of the present invention have been described with specificity in accordance with certain embodiments, the following examples serve only to illustrate the compounds of the invention and are not intended to limit the same.

### Example 1. In Vitro Expression of Human DNAII mRNA

This example illustrates that mRNAs encoding human DNAI1 can successfully translated into DNAI protein in HEK293 T cells.

Various codon-optimized hDNAIl messenger RNAs were synthesized by in vitro transcription from a plasmid DNA template encoding the gene. Table 4 shows various exemplary DNAI1 mRNA constructs prepared. Codon-optimized sequences are indicated by “CO”, and various tagged versions were prepared. Some of the codon-optimized DNAI sequences were further processed by applying a motif screen filter, guanine-cytosine (GC) content analysis filter, and codon adaptation index (CAI) analysis filter, to generate an updated list of optimized nucleotide sequences. These sequences are denoted by “KT” in Table 4 (e.g., Constructs F, G, and K). mDnaic1 is the murine homolog of human DNAI1, which is mutated in approximately 10% of human PCD cases.

TABLE 4

Exemplary DNAI1 mRNA constructs

| Construct | Description | Species | SEQ ID NO |
|---|---|---|---|
| A | CO-DNAI1 | Human | 6 |
| B | CO-DNAI1 | Human | 7 |
| C | DNAI1-FLAG | Human | 12 |
| D | DNAI1-HA | Human | 13 |
| E | DNAI1-stop | Human | 14 |
| F | CO-DNAI1-KT1 | Human | 8 |
| G | CO-DNAI1-KT2 | Human | 9 |
| H | mDnaic1 | Mouse | 11 |
| I | mDnaic1-FLAG | Mouse | 15 |
| J | mDnaic1-HA | Mouse | 16 |
| K | CO-DNAI1-KT3 | Human | 10 |

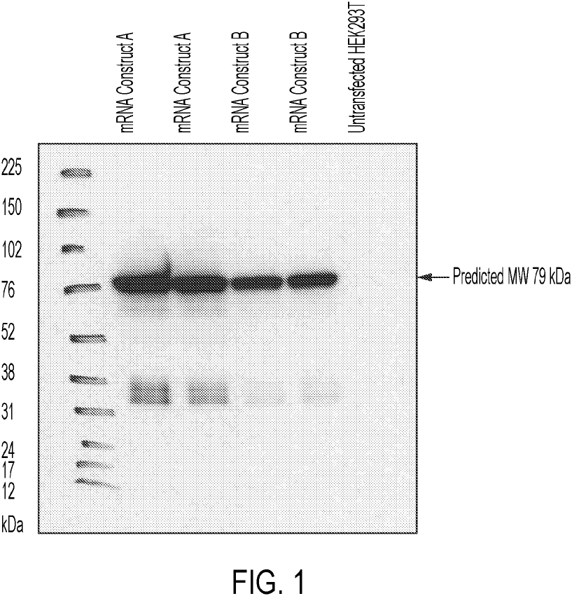

2 ug of mRNA was transfected per 10⁵HEK293T cells. Cells were lysed and prepared for Western blot analysis. 15 µl was loaded per lane and the blot was probed with anti-DNAI1 ab 166912 from Abeam. Untransfected HEK293T cells were also loaded as a negative control. As shown in FIG. 1, high expression of DNAI1 protein was detected for all the mRNA constructs tested.

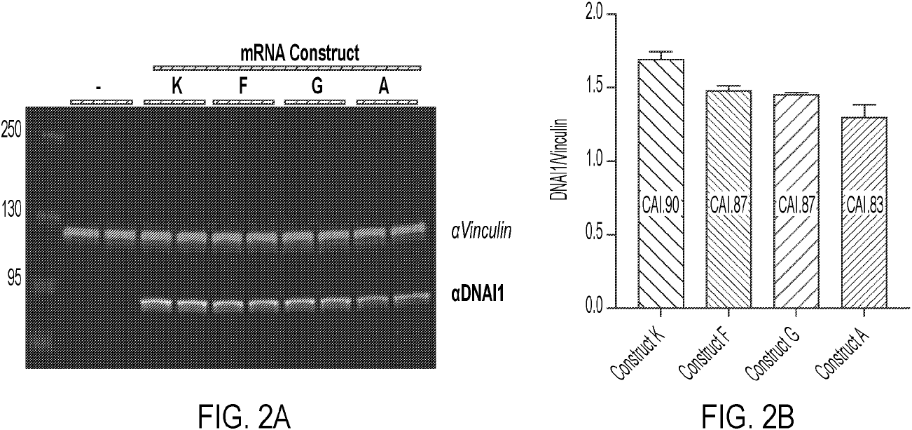

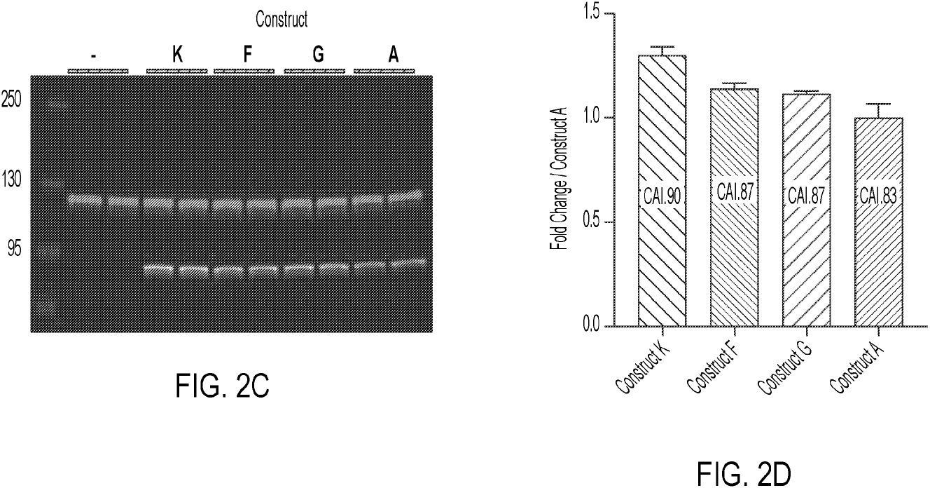

Next, further codon-optimized mRNA sequences (Constructs F, G, and K), which were filtered to for a higher codon adaptation index (CAI) were compared with a reference codon-optimized sequence of Construct A. mRNAs of Constructs A, F, G, or K was each transfected to 10⁵HEK293T cells. Cells were lysed and prepared for Western blot analysis. 0.25 µg was loaded per lane and the blot was probed with anti-DNAI1 antibody and anti-vinculin, as a loading control. Untransfected HEK293T cells were also loaded as a negative control. As shown in FIG. 2A, high expression of DNAI1 protein was detected for all the mRNA constructs tested, and particularly high protein expression for construct K. The ratio of DNAI1/Vinculin protein expression was plotted in FIG. 2B to adjust for loading control. As shown in FIG. 2B, further codon-optimized mRNA sequences (constructs K, F, and G) had higher DNAI1 protein expression. Particularly, construct K, which has a high CAI of about 0.90 outperformed a construct A, which had a CAI of 0.83. Additional Western blot data and a graph showing expression levels of various codon-optimized constructs are in comparison to Construct A are shown in FIGS. 2C and 2D.

### Example 2. In Vivo Administration and Delivery of hDNAI1 mRNA to the Lung and Expression of hDNAI1 Protein

This example illustrates successful in vivo delivery of hDNAI1 mRNA to mice and expression of hDNAI1 protein after a single administration.

The formulations described in the following Examples, unless otherwise specified, contain a multi-component lipid mixture of varying ratios employing one or more cationic lipids, helper lipids (e.g., non-cationic lipids and/or cholesterol lipids) and PEGylated lipids designed to encapsulate human DNAI1 mRNA. Various lipid nanoparticles (LNPs) containing different cationic lipids were tested, as shown in Table 5.

TABLE 5

Exemplary formulations for encapsulation of hDNAI1 mRNA

| Formulation | Cationic Lipid |
|---|---|
| 1 | TL1-04D-DMA |
| 2 | GL-TES-SA-DME-E18-2 |
| 3 | Guan-SS-Chol |
| 4 | SY-3-E14-DMAPr |
| 5 | Cationic lipid 1 |
| 6 | RL3-07D-DMA |
| 7 | TL1-01D-DMA |
| 8 | Cationic lipid 2 |

In this particular example, FLAG-tagged hDNAI1 mRNA was used to differentiate between the endogenous mouse DNAI1 protein.

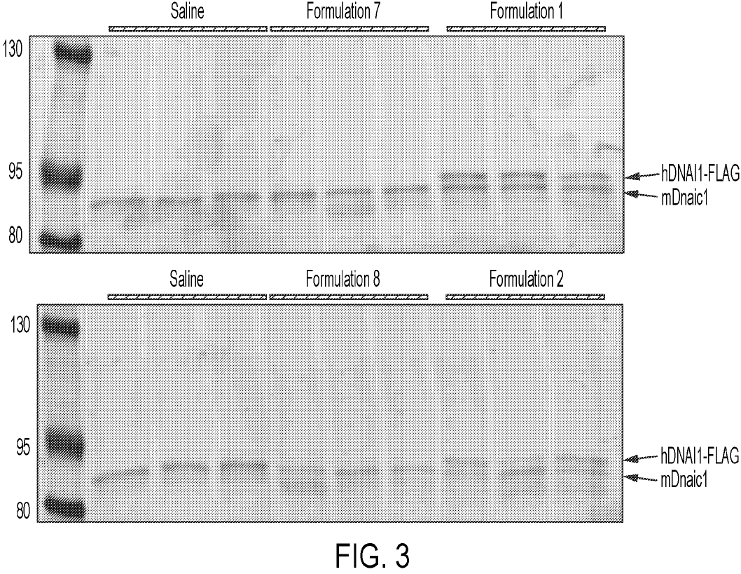

Mice were administered with hDNAI1 mRNA encapsulated within LNPs of formulations 1-8 by intratracheal delivery. As a negative control, saline was administered at the same volume. Mice were euthanized at 48 hours post administration, and the lungs of each mouse was harvested for analysis. FIG. 3 is an exemplary Western blot of mice lungs, detected with anti-DNAI1 antibodies, for 4 of the formulations (plus a negative control) tested. As shown, various formulations comprising different cationic lipids could be used to deliver mRNAs encoding hDNAI1. FIG. 3 shows that the mRNAs encoding hDNAI1 could successfully be translated and expressed in mice, and the protein expression was detectable in the lung even after 48 hours post a single administration.

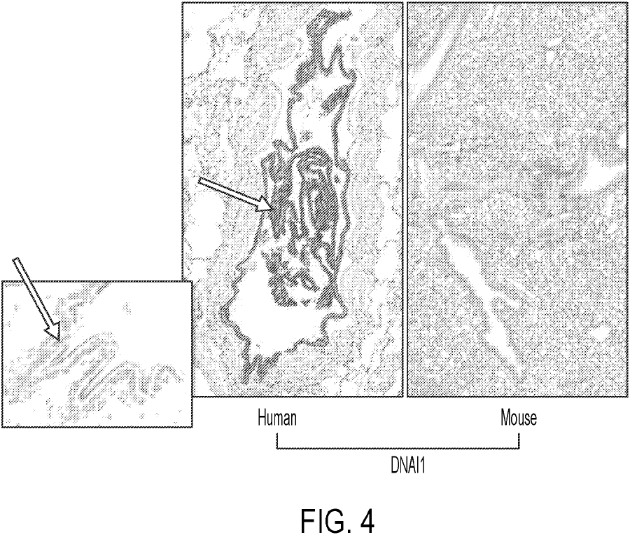

The hDNAI1 protein in the lung bronchus and bronchioles of mice was characterized by IHC staining. Since the anti-DNAI1 antibody detects both human and mouse DNAI1 protein, serial dilution experiment was performed to determine an antibody dilution that detects human DNAI1 protein, but not the mouse protein. Results showed that dilutions of the anti-DNAI1 antibody greater than 1:17000 detects human DNAI1 protein encoded by the delivered mRNA (as indicated by arrows), but not the endogenous mouse DNAI1 (FIG. 4).

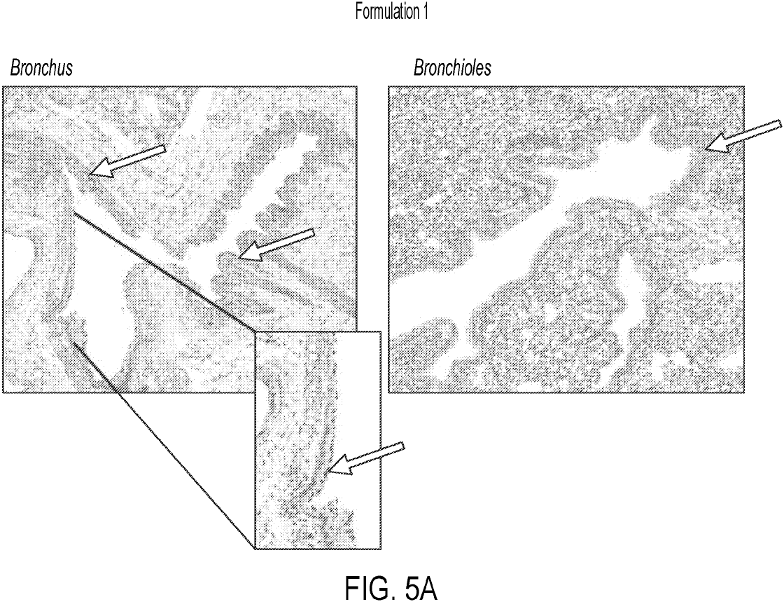

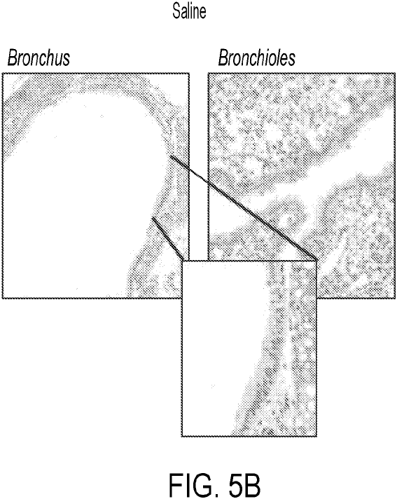

The harvested bronchus and bronchioles tissues of mice, 48 hours after a single intratracheal administration of DNAI1 mRNA encapsulated in LNPs, were characterized by IHC staining with the optimized antibody dilution. FIG. 5A shows an exemplary IHC staining of bronchus and bronchioles of mice administered with formulation 1. The results showed that human DNAI1 protein expression was detected in these tissues after 48 hours of a single dose. Notably, DNAI1 expression was localized to pseudostratified columnar epithelium and apical surfaces, where cilia are located. Arrows indicate exemplary sites showing expression of DNAI1 protein. FIG. 5B shows an IHC staining of bronchus and bronchioles of mice administered with saline, as a negative control. As expected, hDNAI1 protein expression was not detected.

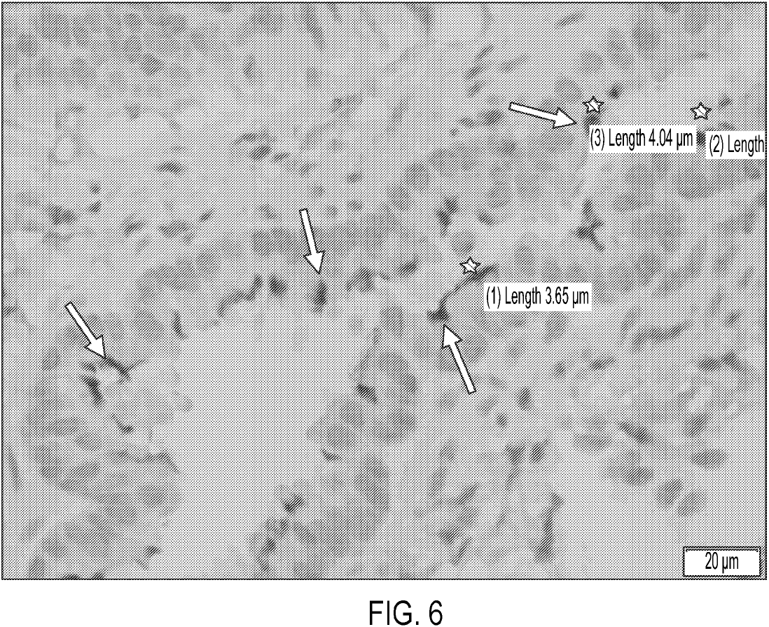

Higher magnification (40x) of the IHC staining showed positive staining through the entire length of cilia, suggesting effective expression and transport of the hDNAI1 protein along the length of cilia (FIG. 6). This is significant because PCD patients characterized by immotile respiratory cilia have DNAI1 protein in bottom half of cilia, resulting in failure of mucus clearance. Therefore, to effectively treat the PCD patients, not only should the DNAI1 protein be expressed, but also be incorporated throughout the entire length of cilia for proper function.

Overall, this example shows the successful in vivo administration, delivery and expression of mRNA encoding hDNAI1. hDNAI1 mRNA could be delivered in vivo encapsulated in various LNPs comprising different cationic lipids. Notably, the present invention allows for successful in vivo delivery and expression of DNAI1 mRNA in lungs and incorporation of the expressed DNAI1 along the entire length of cilia.

Additional studies were performed comparing various codon-optimized DNAI1 mRNA constructs. Table 6 below shows the design of these additional studies, including dosage and concentration used in each of the study arms. Briefly, mice were administered Construct A (SEQ ID NO: 6) and Construct K (SEQ ID NO: 10) each in the same formulation, intratracheally by catheter followed by harvesting of lung tissue 48 hours post administration. The harvested left lung tissues were fixed with 10% NBF overnight and transferred to 70% EtOH at 18-24 h, trachea washed, fixed with 10% NBF and transferred to 70% EtOH at 18-24 h (separate tubes). The harvested right lung tissue were snap frozen.

The data from these studies show robust expression in the lung of both codon-optimized constructs (FIG. 2B). A comparison of normalized DNAI1 expression showed that Construct K had higher expression in the lung tissue (FIG. 2D).

TABLE 6

design of additional in vivo studies

| gRPs | # Animal s/grp | Treatment | Dose level µg/An | Conc (mg/mL) | Dose Volume (uL/An.) | Dosing Regimen | Terminal time point |
|---|---|---|---|---|---|---|---|
| 1 | 5 | Saline | 0 | 0 | 50 | Intratracheal by Catheter on day 1 | At 48 hours post- dose on day 3 |
| 2 | 5 | Construct A | 15 | 0.3 | 50 | | |
| 3 | 5 | Construct K | 15 | 0.3 | 50 | | |

Additional studies were performed to confirm that the codon-optimized mRNA sequence of Construct K results in a robust in vivo protein expression. The mRNA sequence of Construct K was prepared from different DNA plasmids with different backbone structure via in vitro transcription (IVT), Construct K-1 and Construct K-2, respectively.

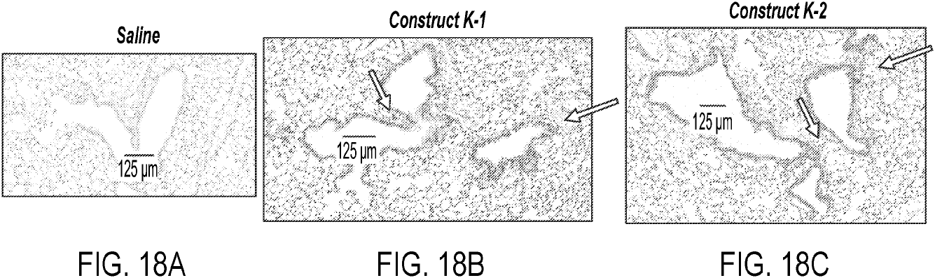

mRNA of Construct K-1 and Construct K-2 were encapsulated into LNPs comprising SY-3-E14-DMAPr as a cationic lipid. Five mice were each administered with hDNAI1 mRNA Construct K-1 or Construct K-2 encapsulated within LNPs by intratracheal delivery. As a negative control, saline was administered at the same volume. Mice were euthanized at 48 hours post administration, and the lungs of each mouse was harvested for analysis. The harvested bronchus and bronchioles tissues of mice, 48 hours after a single intratracheal administration of DNAI1 mRNA encapsulated in LNPs, were characterized by IHC staining with the optimized antibody dilution. FIG. 18B and FIG. 18C are exemplary IHC staining of bronchus and bronchioles of mice administered with LNPs comprising construct K-1 and K-2, respectively. As shown, formulations comprising either Construct K-1 or K-2 showed robust, localized expression of DNAI1 in bronchiolar epithelium post a single administration. Notably, DNAI1 expression was localized to pseudostratified columnar epithelium and apical surfaces, where cilia are located. Arrows indicate exemplary sites showing expression of DNAI1 protein. FIG. 18A shows an IHC staining of bronchus and bronchioles of mice administered with saline, as a negative control. As expected, hDNAI1 protein expression was not detected.

This experiment shows that the codon-optimized mRNA sequence of Construct K is effective in inducing high expression of DNAI1 localized to pseudostratified columnar epithelium and apical surfaces, where cilia are located.

Exemplary codon-optimized mRNA sequences are shown in SEQ ID NO: 6-10. For the purpose of the sequence disclosure, U and T are used interchangeably.

### Example 3. In Vivo Dose Response and Duration of hDNAI1 mRNA Expression

This example illustrates a dose-dependent and time-course study of in vivo hDNAI1 expression.

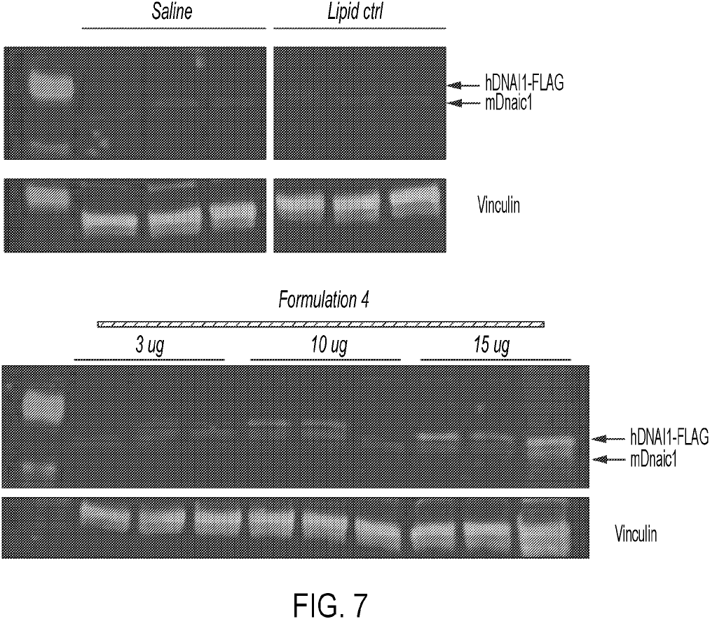

Mice were administered with 3 µg, 10 µg, or 15 µg of hDNAI1 mRNA encapsulated within LNPs of formulations 1 or 4 by intratracheal delivery. As a negative control, saline was administered at the same volume. Mice were euthanized at 48 hours post administration, and the lungs of each mouse was harvested for analysis. FIG. 7 is an exemplary Western blot of lung cells of mice administered with various amounts of formulation 4. Similar results were observed with formulation 1. The results showed the dose-dependent expression of the hDNAI1 protein encoded by the delivered mRNA.

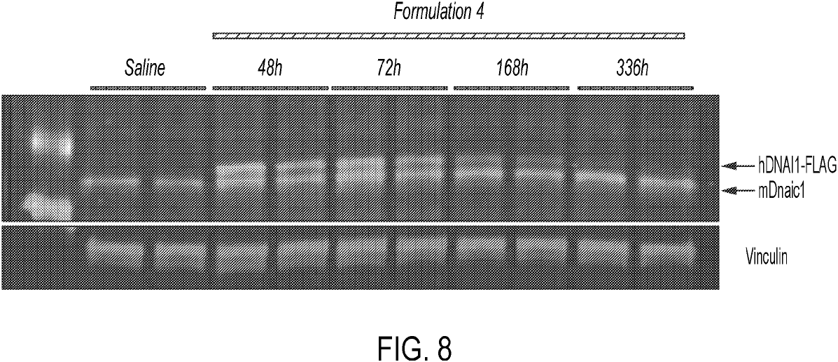

Mice were administered with hDNAI1 mRNA encapsulated within LNPs of formulations 1 or 4 by intratracheal delivery. As a negative control, saline was administered at the same volume. Mice were sacrificed at 48, 72, 168, or 336 hours post administration, and the lungs of each mouse was harvested for analysis. FIG. 8 is an exemplary Western blot of lung cells of mice administered with formulation 4 at various time points. Similar results were observed with formulation 1. The results show that strong hDNAI1 protein expression was detectable at 168 hour (7 days) post single administration of the mRNA-LNP. Notably, hDNAI1 protein was detectable even after 336 hours (14 days) post administration.

### Example 4. Synthesis of Lipids for Use in Pulmonary Delivery

Synthesis of cationic lipids of the present invention are described in this example.

### 1. Gl-tes-sa-dme-e18-2

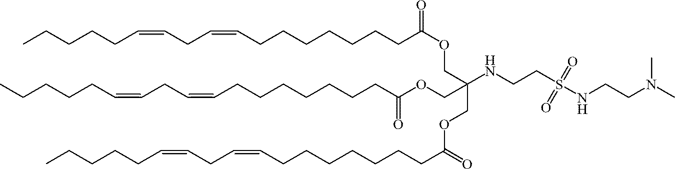 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCN(C)C]

### Synthetic Scheme

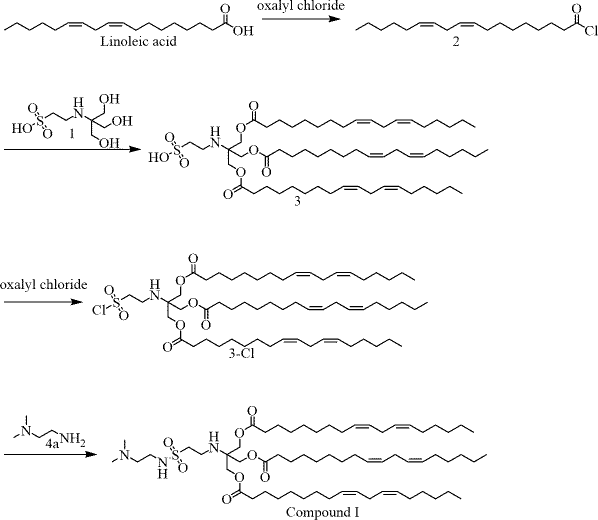

### Synthetic Protocol

### Synthesis of (9Z,12Z)-Octadeca-9,12-dienoyl Chloride (2)

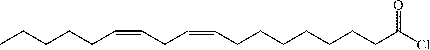 [SMILES: CCCCC/C=C\C/C=C\CCCCCCCC(=O)Cl]

To a solution of Linolenic acid (1.0 g, 3.6 mmol) in 10 mL dichloromethane at 0° C., was added N, N-dimethylformamide (0.1 mL) and oxalyl chloride (1.2 mL, 14.3 mmol). The reaction mixture was warmed to room temperature and stirred for 3 h. The solvent was removed to the under reduced pressure, and the crude was used in next step without further purification.

### Synthesis of 2-((1,3-Bis(((9Z,12Z)-octadeca-9,12-dienoyl)oxy)-2-((((9Z,12Z)-octadeca-9,12-dienoyl)oxy)methyl)propan-2-yl)amino)ethane-1-sulfonic Acid (3)

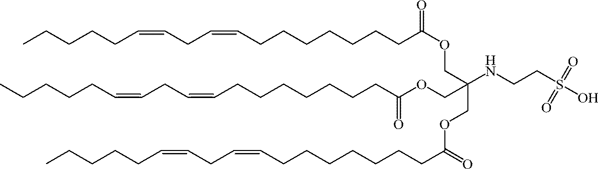 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)O]

To a solution of (9Z,12Z)-octadeca-9,12-dienoyl chloride 2 (1.1 g, 3.6 mmol) in anhydrous N,N-dimethylacetamide (5.0 mL) and N-methyl morpholine (3.0 mL), was added 2-((1,3-dihydroxy-2-(hydroxymethyl)propan-2-yl)amino)ethane-1-sulfonic acid (1, TES) (200 mg, 0.87 mmol). The reaction mixture was heated to 55° C. for 3 h. MS analysis showed the formation of desired product. The reaction mixture was cooled to room temperature, diluted with water (100 mL) and extracted with dichloromethane (2 x 100 mL). The combined organic layer was washed with saturated brine (100 mL) and dried over anhydrous sodium sulfate. The solvent was removed under vacuum, and the residue was purified by column chromatography (40 g SiO₂: 0 to 10% methanol in dichloromethane gradient) to obtain 2-((1,3-bis(((9Z,12Z)-octadeca-9,12-dienoyl)oxy)-2-((((9Z,12Z)-octadeca-9,12-dienoyl)oxy)methyl)propan-2-yl)amino)ethane-1-sulfonic acid as colorless solid (562 mg, 47% yield).

### Synthesis of 2-((2-(Chlorosulfonyl)ethyl)amino)-2-((((9Z,12Z)-octadeca-9,12-dienoyl)oxy)methyl)propane-1,3-diyl (9Z,9′Z,12Z,12′Z)-bis(octadeca-9,12-dienoate) (3-Cl)

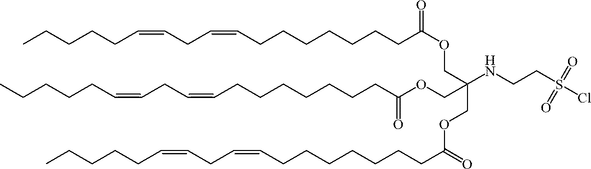 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)Cl]

To a solution of 2-((1,3-bis(((9Z,12Z)-octadeca-9,12-dienoyl)oxy)-2-((((9Z,12Z)-octadeca-9,12-dienoyl)oxy)methyl)propan-2-yl)amino)ethane-1-sulfonic acid 3 (210 mg, 0.82 mmol) in

anhydrous dichloromethane (5.0 mL) at 0° C. was added N, N-dimethylformamide (0.05 mL) and oxalyl chloride (0.08 mL, 2.1 mmol). The reaction mixture was warmed to room temperature and stirred for 3 h. The solvent was removed to the dryness under reduced pressure to give 2-((2-(chlorosulfonyl)ethyl)amino)-2-((((9Z,12Z)-octadeca-9,12-dienoyl)oxy)methyl)propane-1,3-diyl (9Z,9′Z,12Z,12′Z)-bis(octadeca-9,12-dienoate), which was used in next step without further purification.

### Synthesis of 2-((2-(N-(2-(dimethylamino)ethyl)sulfamoyl)ethyl)amino)-2-((((9Z,12Z)-octadeca-9,12-dienoyl)oxy)methyl)propane-1,3-diyl (9Z,9′Z,12Z,12′Z)-bis(octadeca-9,12-dienoate) (Compound I)

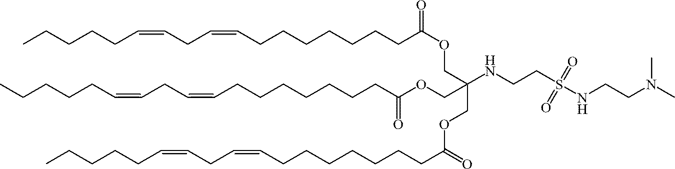 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCN(C)C]

To a solution of 2-((2-(chlorosulfonyl)ethyl)amino)-2-((((9Z,12Z)-octadeca-9,12-dienoyl)oxy)methyl)propane-1,3-diyl (9Z,9′Z,12Z,12′Z)-bis(octadeca-9,12-dienoate) 3-Cl (210 mg, 0.82 mmol) in anhydrous dichloromethane (5.0 mL) at 0° C. was added N1,N1-dimethylethane-1,2-diamine (182 mg, 2.1 mmol). The reaction mixture was warmed to room temperature and stirred for 3 h. The reaction was quenched by addition of water, and the mixture was extracted with dichloromethane (2 x 100 mL). The combined organic layer was washed with saturated brine (100 mL) and dried over anhydrous sodium sulfate. The solvent was removed, and the crude was purified by column chromatography (40 g SiO2— 0 to 15% methanol in dichloromethane gradient) to obtain 2-((2-(N-(2-(dimethylamino)ethyl)sulfamoyl)ethyl)amino)-2-((((9Z,12Z)-octadeca-9,12-dienoyl)oxy)methyl)propane-1,3-diyl (9Z,9′Z,12Z,12′Z)-bis(octadeca-9,12-dienoate) as yellow oil (139 mg, 62% yield).

1H NMR (300 MHz, Chloroform-d) δ 5.26-5.44 (m, 12H), 4.09 (s, 6H), 3.06-3.18 (m, 6H), 2.75 (t, 6H), 2.47 (t, 2H), 2.32 (t, 6H), 2.24 (s, 6H), 2.00-2.10 (m, 12H), 1.52-1.65 (m, 4H), 1.20-1.40 (m, 44H), 0.88 (t, 9H).

APCI-MS analysis: Calculated C64H115N3O8S, [M+H] = 1186.7, observed = 1186.8.

### 2. Gl-tes-sa-dmp-e18-2

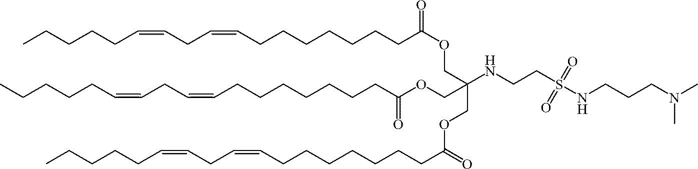 [SMILES: CCCCC/C=C\C/C=C\CCCCCCCC(=O)OCC(COC(=O)CCCCCCC/C=C\C/C=C\CCCCC)(COC(=O)CCCCCCC/C=C\C/C=C\CCCCC)NCCS(=O)(=O)NCCCN(C)C]

### Synthetic Pathway

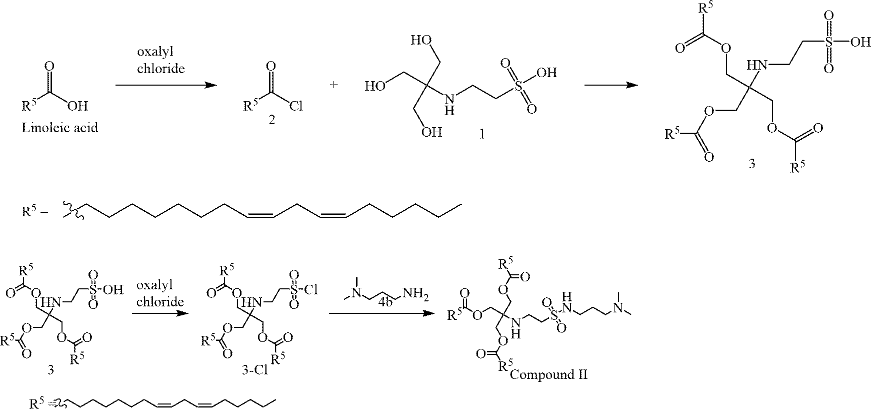

### Synthetic Protocol

Compound II was prepared following the above representative procedure in similar yields to those obtained for Compound I.

Linoleic acid is treated with a chlorinating reagent such as oxalyl chloride to provide the acyl chloride compound 2. Reaction of compound 2 with a nucleophilic compound, such as the buffer compound 1, affords compound 3. Compound 3 is treated with a chlorinating agent such as oxalyl chloride to provide the electrophilic compound 3-Cl. Reaction of 3-Cl with a nucleophile such as compound 4b then affords compound II.

The reaction conditions used were as follows:

| | | | | | | |
|---|---|---|---|---|---|---|
| | SM | Product | Reaction Conditions | Scale | Yield | |
| 1H | Linoleic acid | 2 | Oxalyl chloride, DMF DCM | 1.0 g of linoleic acid | - | NMR |
| (300 | 1&2 | 3 | Dimethylacetamide + N-Methylmorpholine | 200 mg of 1 | 562 mg 47% yield | MHz, |
| | 3 | 3-C1 | Oxalyl chloride, DMF DCM | 200 mg of 3 | - | |
| | 3-Cl & 4b | Compound II | DCM | 200 mg of 3-C1 | 105 mg (49% over 2 steps) | |

Chloroform-d) δ 5.24-5.42 (m, 12H), 4.08 (s, 6H), 3.17 (t, 2H), 3.06 (bs, 4H), 2.75 (t, 6H), 2.43 (t, 2H), 2.31 (t, 6H), 2.23 (s, 6H), 1.98-2.08 (m, 12H), 1.70 (quint, 2H), 1.52-1.63 (m, 4H), 1.17-1.45 (m, 44H), 0.87 (t, 9H).

APCI-MS analysis: Calculated C65H117N3O8S, [M+H] = 1100.7, observed = 1100.8.

### 3. Tl1-01d-dma

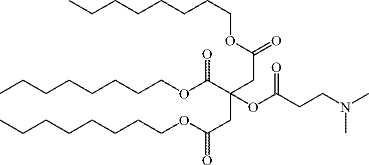 [SMILES: CCCCCCCCOC(=O)CC(CC(=O)OCCCCCCCC)(OC(=O)CCN(C)C)C(=O)OCCCCCCCC]

### Synthetic Scheme

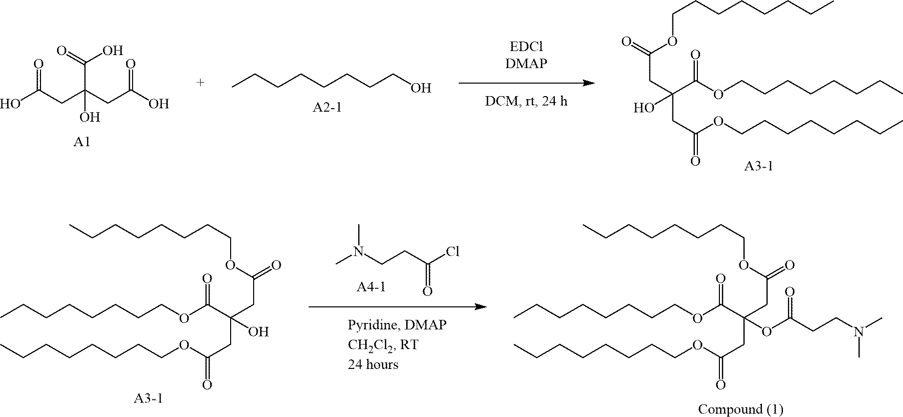

### Synthetic Protocol

### Synthesis of (trioctyl 2-hydroxypropane-1,2,3-tricarboxylate)

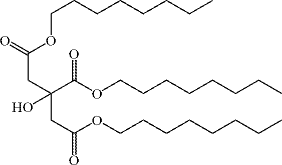 [SMILES: CCCCCCCCOC(=O)CC(O)(CC(=O)OCCCCCCCC)C(=O)OCCCCCCCC]

To a solution of citric acid A1 (2.1 g, 11.0 mmol) and 1-octanol A2-1 (9.4 g, 72.6 mmol) in dichloromethane (40 mL), DMAP (1.34 g, 11.0 mmol) and EDCI (14.3 g, 72.6 mmol) were added, and the resulting mixture was stirred at room temperature 24 h. The reaction mixture was evaporated under vacuum. The residue was dissolved in dichloromethane (200 mL) and washed with brine (100 mL x 3). After dried over anhydrous Na₂SO₄, the solvent was evaporated, and the crude was purified by column chromatography (220 g SiO₂— 0 to 20% ethyl acetate in hexane gradient) to obtain (trioctyl 2-hydroxypropane-1,2,3-tricarboxylate) as colorless oil (5.2 g, 90%).

### Synthesis of (trioctyl 2-((3-(dimethylamino)propanoyl)oxy)propane-1,2,3-tricarboxylate)

To a solution of trioctyl 2-hydroxypropane-1,2,3-tricarboxylate A3-1 (0.528 g, 1.0 mmol), DMAP (122 mg, 1.0 mmol) and pyridine (316 mg, 4.0 mmol) in 10 mL dichloromethane, 3-(dimethylamino)propanoyl chloride A4-1 (271 mg, 2.0 mmol) was added at 0° C., and then the resulting mixture was stirred at room temperature for 24 h. The reaction mixture was evaporated under vacuum. The residue was dissolved in dichloromethane (100 mL) and washed with brine (80 mL x 3). After dried over anhydrous Na₂SO₄, the solvent was evaporated, and the crude was purified by column chromatography (80 g SiO₂— 0 to 10% methanol in dichloromethane gradient) to obtain trioctyl 2-((3-(dimethylamino)propanoyl)oxy)propane-1,2,3-tricarboxylate as colorless oil (210 mg, 33%).

Alternatively, to a suspension of 3-(dimethylamino)propanoic acid (8.02 g, 68.5 mmol) in 150 mL dichloromethane, was added EDCI (13.1 g, 68.5 mmol) and DMAP (2.09 g, 17.1 mmol) at 0° C., and the resulting mixture was stirred at this temperature for 5 min. A solution of trioctyl 2-hydroxypropane-1,2,3-tricarboxylate A3-1 (9.05 g, 17.1 mmol) in 10 mL dichloromethane was added, and then the resulting mixture was stirred at room temperature for 48 h. The reaction mixture was diluted with dichloromethane, washed with saturated sodium bicarbonate and brine. After dried over sodium sulfate, the organic layer was evaporated under vacuum. The residue was purified by column chromatography (220 g SiO2— 0 to 10% methanol in dichloromethane gradient) to obtain trioctyl 2-((3-(dimethylamino)propanoyl)oxy)propane-1,2,3-tricarboxylate as colorless oil (4.2 g, 38%).

¹H NMR (300 MHz, CDCl₃) δ 4.56 (s, br., 6H), 4.24 (t, 2H), 4.12 (s, 2H), 2.55 (t, 2H), 2.28-2.17 (m, 14H), 1.63-1.48 (m, 8H), 1.25 (s, br., 32H), 0.86 (t, 12H).

APCI-MS analysis: Calculated C35H65NO8, [M+H] = 627.9, Observed = 628.5.

### 4. Tl1-04d-dma

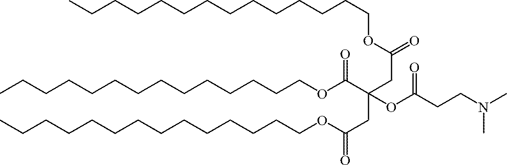 [SMILES: CCCCCCCCCCCCCCOC(=O)CC(CC(=O)OCCCCCCCCCCCCCC)(OC(=O)CCN(C)C)C(=O)OCCCCCCCCCCCCCC]

TD1-04D-DMA can be made in a similar manner as TD-01D-DMA, which is described above.

### 5. SYE14-DMAPr

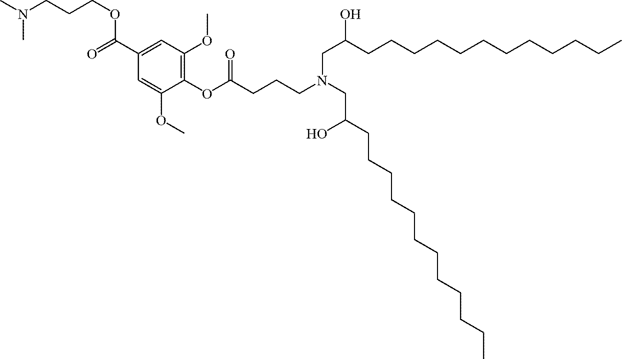 [SMILES: CCCCCCCCCCCCC(O)CN(CCCC(=O)Oc1c(OC)cc(C(=O)OCCCN(C)C)cc1OC)CC(O)CCCCCCCCCCCC]

### Synthetic Scheme

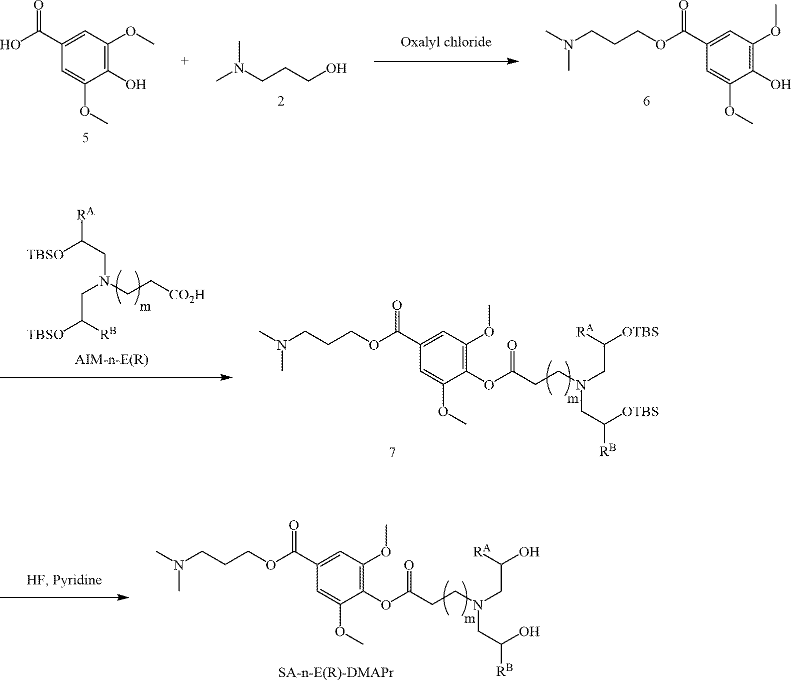

R^{A} ═ R^{B} ═ C₁₂H₂₅

### Synthesis of 3-(Dimethylamino)propyl 4-hydroxy-3,5-dimethoxybenzoate (6)

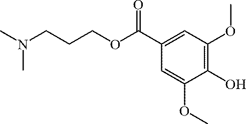 [SMILES: COc1cc(C(=O)OCCCN(C)C)cc(OC)c1O]

To a suspension of syringic acid 5 (7.5 g, 0.04 moles) in 100 mL dichloromethane at 0° C. was added oxalyl chloride (12.8 mL, 0.15 mole) followed by dimethylformamide (5 drops), and the resulting mixture was stirred for 2 h at this temperature. The reaction mixture was evaporated to dryness, and the residue was dissolved in 100 mL dichloromethane. After cooling to 0° C., 3-(dimethylamino)propan-1-ol 2 (4.5 mL, 40 mmol) was added slowly, and the reaction mixture was stirred at room temperature overnight. The precipitate was filtered to give 3-(dimethylamino)propyl 4-hydroxy-3,5-dimethoxybenzoate 6 as white solid (6.2 g, 58%).

### 6. Tl1-10d-dma

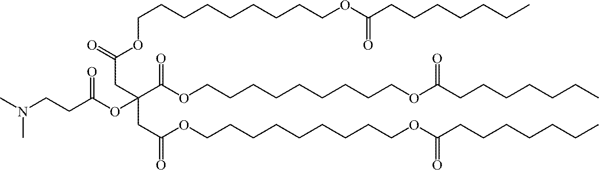 [SMILES: CCCCCCCC(=O)OCCCCCCCCCOC(=O)CC(CC(=O)OCCCCCCCCCOC(=O)CCCCCCC)(OC(=O)CCN(C)C)C(=O)OCCCCCCCCCOC(=O)CCCCCCC]

TD1-04D-DMA can be made in a similar manner as TD-01D-DMA, which is described above.

### 7. Hep-e3-e10

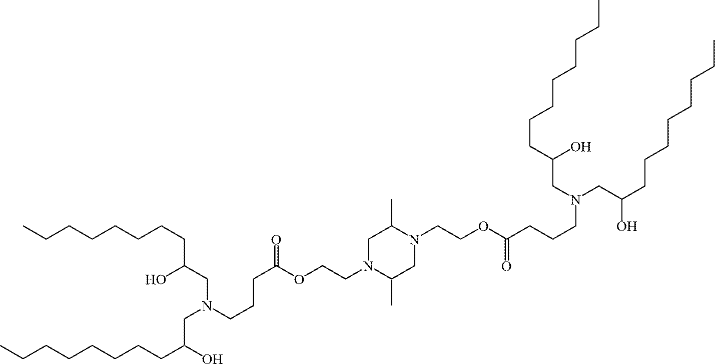 [SMILES: CCCCCCCCC(O)CN(CCCC(=O)OCCN1CC(C)N(CCOC(=O)CCCN(CC(O)CCCCCCCC)CC(O)CCCCCCCC)CC1C)CC(O)CCCCCCCC]

### Synthetic Scheme

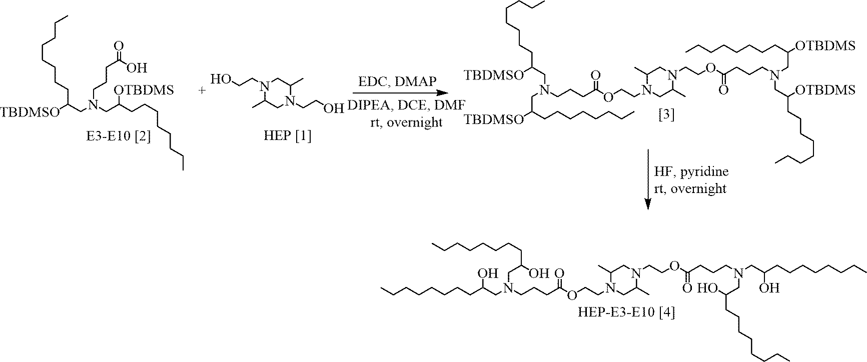

### Synthetic Protocol

### Synthesis of [3]

As set out in Scheme 1: To a solution containing HEP [1] (0.100 g, 0.494 mmol, 1.0 eq), E3-E10 [2] (0.668 g, 1.038 mmol, 2.1 eq), 1ml of dimethylformamide, 3ml of dichloroethane, diisopropylethylamine (0.344 µL, 1.98 mmol, 4.0 eq), and N,N-Dimethylaminopyridine (0.024 g, 0.198 mmol, 0.4 eq) was added 1-Ethyl-3-(3-dimethylaminopropyl)carbodiimide (0.285 g, 1.48 mmol, 3.0 eq) and allowed to react at room temperature overnight (18 hr). Afterwards, the reaction mixture was concentrated using a rotavapor and purified using a Buchi Combi-flash system on 12 g, 40 µm-sized silica gel columns using hexanes/ethyl acetate as the mobile phase, yielding a colorless oil (70% yield).

### Synthesis of HEP-E3-E10 [4]

As set out in Scheme 1: To a 20 ml Polypropylene scintillation vial equipped with a PTFE stir-bar was added [3] (0.500 g, 0.344 mmol, 1.0 eq) along with 4ml of dry tetrahydrofuran. The vial was cooled to 0-5° C. on an ice bath and HF/pyridine (1.76 ml, 67.86 mmol, 197.3 eq) was added dropwise. After addition, the reaction vial was allowed to warm to room temperature and stirred overnight (18 hr). Afterwards, the reaction mixture was neutralized with saturated sodium bicarbonate at 0° C. Ethyl acetate was used for extraction (3x). The organic layers were combined, washed with saturated sodium chloride (4x), dried with sodium sulfate, filtered, and rotovaped to yield an off-yellow oil. This oil was further purified using a Buchi Combi-flash system on 12 g, 40 µm-sized silica gel columns using dichloromethane/methanol (3% methanol) as the mobile phase, yielding a colorless oil (60% yield).

1H NMR (400 MHz, CDCl3) 4.16 (m, 4H), 3.60 (m, 4H), 2.97 (m, 3H), 2.78 (d, 3H), 2.58 (m, 9H), 2.37 (m, 12H), 2.15 (m, 2H), 1.78 (m, 4H), 1.44 (m, 7H), 1.36 (m, 9H), 1.26 (br, 45H), 1.05 (d, 6H), 0.87 (t, 12H).

Expected M/Z = 998.59, Observed = 998.0.

### 8. Hep-e4-e10

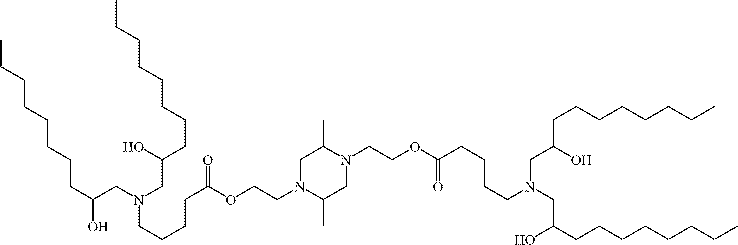 [SMILES: CCCCCCCCC(O)CN(CCCCC(=O)OCCN1CC(C)N(CCOC(=O)CCCCN(CC(O)CCCCCCCC)CC(O)CCCCCCCC)CC1C)CC(O)CCCCCCCC]

### Synthetic Scheme

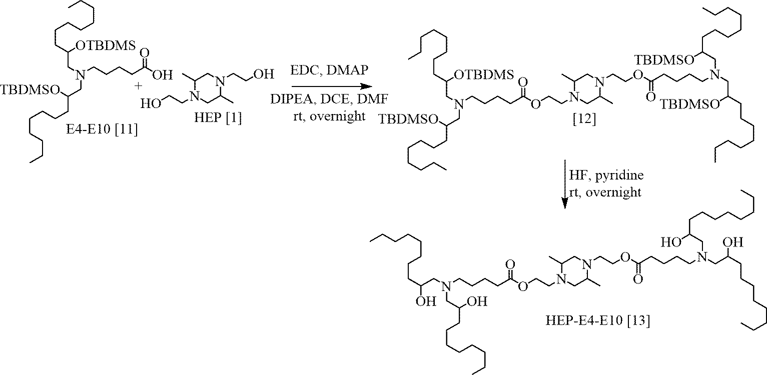

### Synthetic Protocol

### Synthesis of [12]

As set out in Scheme 2: To a solution of HEP [1] (0.100 g, 0.494 mmol, 1.0 eq), E4-E10 [11] (0.683 g, 1.038 mmol, 2.1 eq), 1 ml of dimethylformamide, 3 ml of dichloroethane, diisopropylethylamine (0.344 µL, 1.98 mmol, 4.0 eq), and N,N-Dimethylaminopyridine (0.024 g, 0.198 mmol, 0.4 eq) was added 1-Ethyl-3-(3-dimethylaminopropyl)carbodiimide (0.285 g, 1.48 mmol, 3.0 eq) and allowed to react at room temperature overnight (18 hr). Afterwards, the reaction mixture was concentrated using a rotavapor and purified using a Buchi Combi-flash system on 12 g, 40 µm-sized silica gel columns using hexanes/ethyl acetate as the mobile phase, yielding a colorless oil (63.3% yield).

### Synthesis of HEP-E4-E10 [13]

As set out in Scheme 2: To a 20 ml Polypropylene scintillation vial equipped with a PTFE stir-bar was added [12] (0.450 g, 0.303 mmol, 1.0 eq) along with 4ml of dry tetrahydrofuran. The vial was cooled to 0-5° C. on an ice bath and HF/pyridine (1.55 ml, 59.920 mmol, 197.3 eq) was added dropwise. After addition, the reaction vial was allowed to warm to room temperature and stirred overnight (18 hr). Afterwards, the reaction mixture was neutralized with saturated sodium bicarbonate at 0° C. Ethyl acetate was used for extraction (3x). The organic layers were combined, washed with saturated sodium chloride (4x), dried with sodium sulfate, filtered, and rotovaped to yield an off-yellow oil. This oil was further purified using a Buchi Combi-flash system on 12 g, 40 µm-sized silica gel columns using dichloromethane/methanol (3%) as the mobile phase, yielding a colorless oil (48.4% yield).

1H NMR (400 MHz, CDCl3) 4.16 (t, 4H), 3.62 (br, 4H), 2.96 (q, 3H), 2.76 (d, 4H), 2.56 (m, 8H), 2.40 (m, 4H), 2.32 (t, 4H), 2.13 (t, 2H), 1.61 (m, 4H), 1.46 (m, 8H), 1.37 (m, 8H), 1.28 (br, 44H), 1.03 (d, 6H), 0.87 (t, 12H),

13C NMR (400 MHz, CDCl3) 173.65 (2C), 69.65 (2C), 68.04 (2C), 62.84 (2C), 61.82 (2C), 61.44 (2C), 60.89 (2C), 55.57 (4C), 51.55 (2C), 35.35 (4C), 34.20 (2C), 32.09 (7C), 30.00 (5C), 29.77 (6C), 29.47 (6C), 26.93 (2C), 25.84 (5C), 22.84 (9C), 17.77 (2C), 14.30 (7C).

Expected M/Z = 1025.64, Observed = 1025.8.

### 9. Guan-SS-Chol

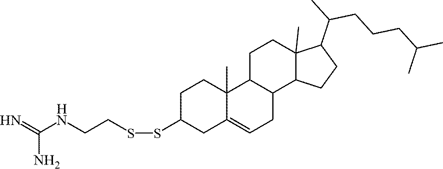 [SMILES: CC(C)CCCC(C)C1CCC2C3CC=C4CC(SSCCNC(=N)N)CCC4(C)C3CCC12C]

Guan-SS-Chol can be made according to methods described in International Publication No. WO 2018/089801, which is hereby incorporated by reference in its entirety. Guan-SS-Chol and Formula (V) (HGT4002) are used interchangeably.

### Example 5. Evaluating Cationic Lipids for Pulmonary Delivery

In this example, various cationic lipids were tested for in vivo efficacy when mRNA encapsulated in lipid nanoparticles (mRNA-LNP) were administered to mice by pulmonary delivery. The cationic lipids were tested for both potency, as determined by levels of protein production, and tolerability, as determined by side effects associated with clearance and metabolism.

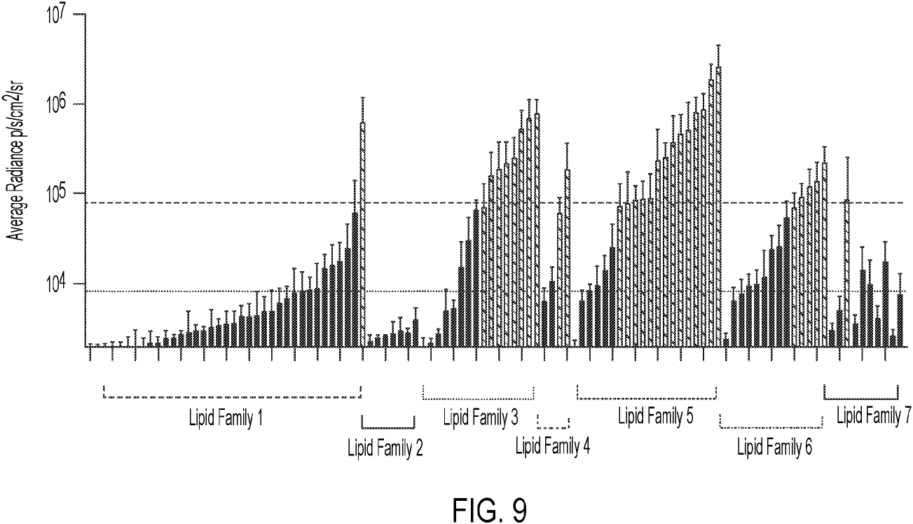

About 150 cationic lipids were tested. (FIG. 9). Each cationic lipid was used in preparing lipid nanoparticles encapsulating mRNA encoding firefly luciferase protein (FFL mRNA) according to methods known in the art. For example, suitable methods for mRNA encapsulation include methods described in International Publication Nos. WO2016/004318 and WO 2018/089801, which is hereby incorporated by reference in its entirety. The tested lipid nanoparticles comprised a lipid component consisting of a cationic lipid, a non-cationic lipid (DOPE), a PEG-modified lipid (DMG-PEG2K), and optionally cholesterol.

Lipid nanoparticle formulations comprising FFL mRNA were administered to male CD1 mice by a single intratracheal administration via nebulization using a Microsprayer®. At approximately 5 hours post-dose, the animals were dosed with luciferin by intraperitoneal injection and all animals were imaged using an IVIS imaging system to measure luciferase production in the lung. FIG. 9 shows that each cationic lipid has various efficacy of in vivo protein expression in the lung. Some cationic lipids remarkably had greater than 50-fold increase in pulmonary protein expression as compared to other cationic lipids.

Based on their performance in this in vivo screen, nine cationic lipids were selected for further investigation: GL-TES-SA-DME-E18-2, TL1-01D-DMA, SY-3-E14-DMAPr, TL1-10D-DMA, HGT4002 (also referred to herein as Guan-SS-Chol), GL-TES-SA-DMP-E18-2, HEP-E4-E10, HEP-E3-E10, and TL1-04D-DMA. Of these, HEP-E4-E10, HEP-E3-E10, GL-TES-SA-DME-E18-2, GL-TES-SA-DMP-E18-2, TL1-01D-DMA and TL1-04D-DMA displayed particularly high potency as determined by the average radiance detected in mouse lungs.

### Example 6. Evaluating Lipid Nanoparticles for Protein Expression

In this example, cationic lipids were tested for both in vivo mRNA delivery and protein expression to evaluate potency and biodistribution. In this study, cationic lipids TL-10D-DMA, SY-3-E14-DMAPr, and TD1-04D-DMA were used to prepare lipid nanoparticles LNP-A, LNP-B, and LNP-C, respectively, encapsulating mCherry mRNA.

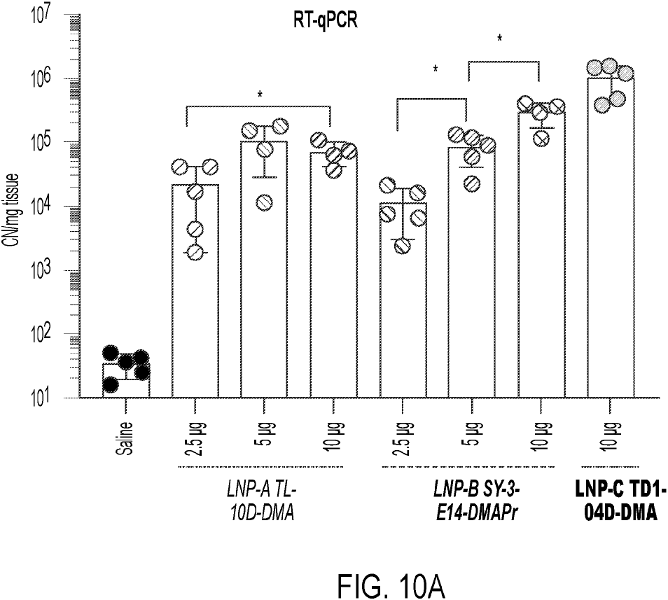

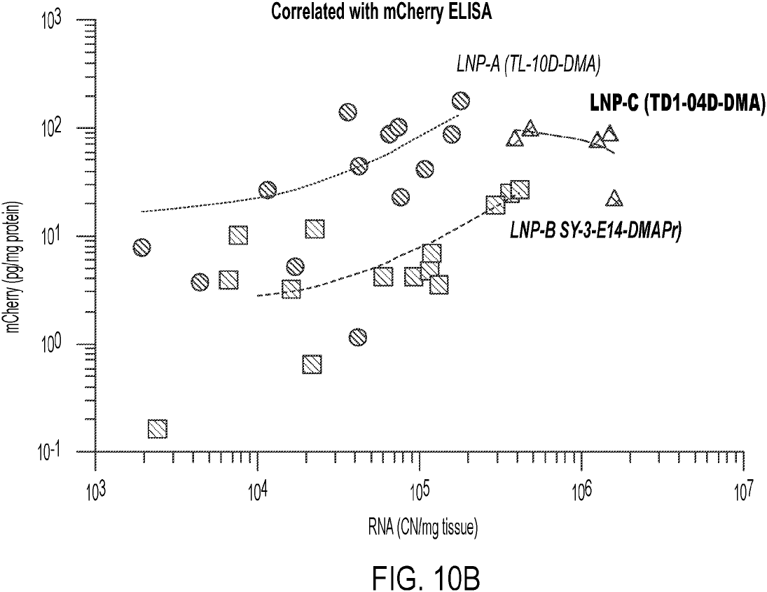

The mRNA-LNPs were administered to mice by intratracheal administration and the amount of mRNA delivered to the lung tissue was determined. As shown in FIG. 10A, all the mRNA-LNPs tested deliver mRNA more effectively to lung cells. To examine whether the amount of mRNA delivered to the lung cells correlates with protein expression in the lung, the amount of mCherry protein was determined by ELISA. FIG. 10B shows the amount of mRNA in the lung tissue in the x-axis and the amount of protein expressed in the lung in the y-axis. The data shows that certain LNPs have higher potency as shown by increased protein expression, even when the same amount of mRNA was delivered to the tissue. For example, for the same amount of 10⁵ CN/mg tissue RNA delivered, LNP-B resulted in about 10 pg/mg protein, whereas LNP-A resulted in about 10²pg/mg protein.

### Example 7. Evaluating Lipid Nanoparticles for Biodistribution by Pulmonary Delivery

In this example, LNPs encapsulating mRNAs were tested for biodistribution when mRNA-LNPs were administered to mice by pulmonary delivery.

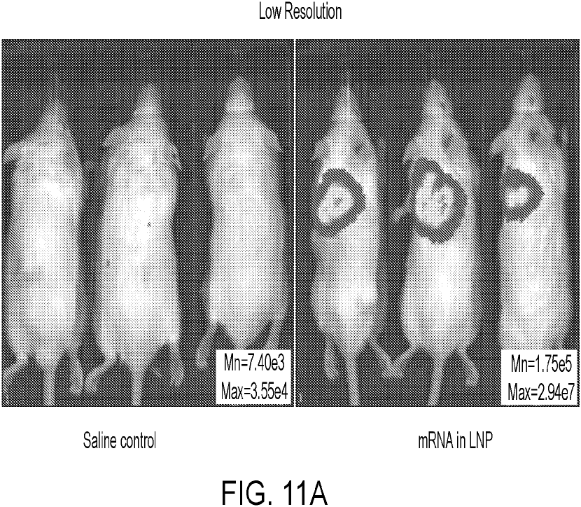

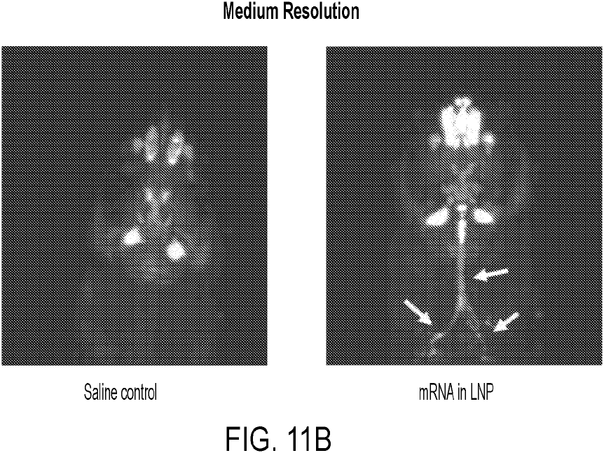

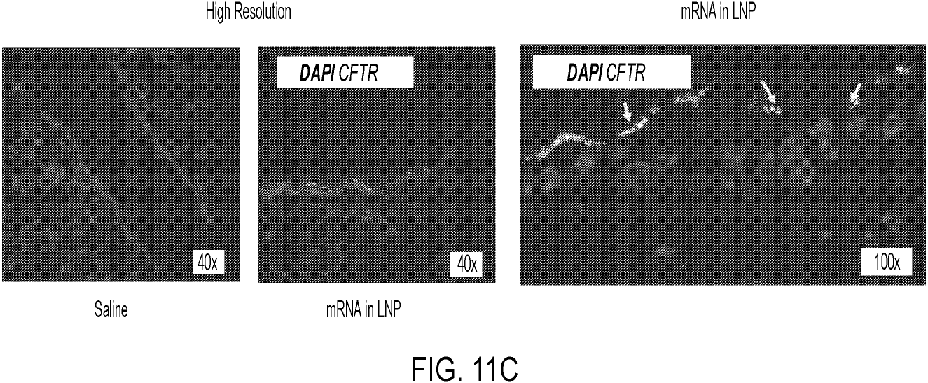

First, a study was done to examine whether the mRNA-LNPs of the present invention are delivered effectively to the lungs in vivo. LNPs encapsulating FFL mRNA was administered to CD-1 mice by intratracheal delivery, and radiance was detected at 24 hours post-administration. As shown in FIG. 11A, the results demonstrate that mRNA-LNPs were effectively delivered to the lungs of mice.

To identify which types of cells the mRNA-LNPs transfect in vivo, genetically modified mice were used whose cells, after successful transfection with Cre recombinase, express fluorescent tdTomato protein. Following in vivo administration of mRNA encoding Cre recombinase, it is possible to visualize successfully transfected cells in bulk tissues with single-cell resolution by detecting Cre-induced tdTomato expression. LNPs encapsulating Cre recombinase mRNA was administered to tdTomato transgenic mice by nebulization. After 48 hours, mice were imaged by Cryofluorescence Tomography. FIG. 11B shows that mRNA-LNPs were delivered effectively to the lung, and protein expression was observed even in the branches of the airway, as indicated by the arrows.

Next, to examine biodistribution and expression of mRNA-LNPs at high resolution, LNPs encapsulating CFTR mRNA were prepared. mRNA-LNPs were administered to CFTR knock-out (KO) mice by pulmonary delivery. Protein expression was detected by immunofluorescence. FIG. 11C shows that the CFTR proteins expressed by the delivered mRNA-LNPs were present on the apical surface of the airways, as indicated by the arrows, demonstrating the effectiveness of the mRNA-LNPs of the present invention.

### Example 8. Evaluating Lipid Nanoparticles for Protein Expression by HBEC-ALI

In this example, LNPs encapsulating mRNAs were tested for protein expression using the HBEC-ALI (Human Bronchial Epithelial Cell - Air Liquid Interface) system. HBEC-ALI technique is advantageous as it reproduces a well differentiated airway epithelium with distinct, functional cells, allowing it to be used as a highly translatable airway cell model.

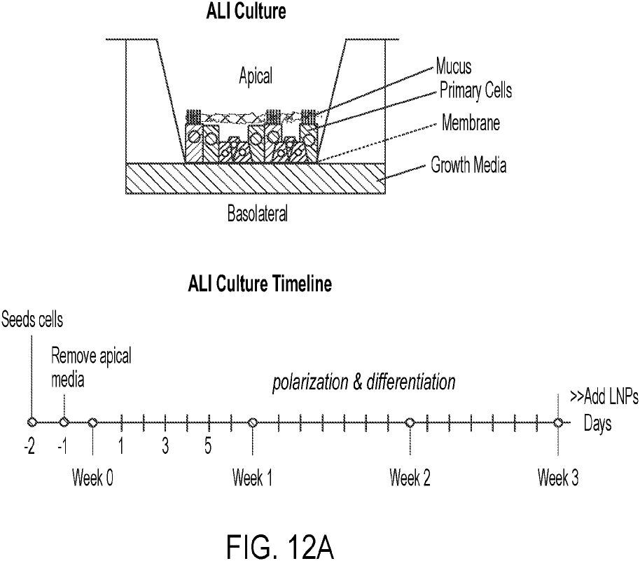

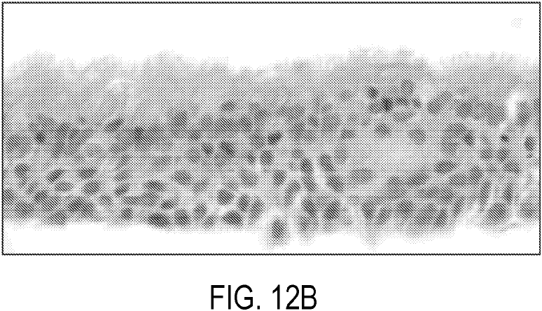

Obtaining a successful HBEC-ALI culture that can be used in future experiments is critical. Briefly, human bronchial epithelial cells were seeded on wells and grown in media. Upon reaching confluency, the apical media was removed and replaced with growth culture media. Cells were grown to allow polarization and differentiation before experiments with mRNA-LNPs were performed, as shown in FIG. 12A. The exemplary HBEC-ALI system schematic is shown in FIG. 12A. The differentiated epithelium were sectioned and stained with hematoxylin and eosin (H&E), as shown in FIG. 12B, which indicates the presence of multi-ciliated cells that can be used as airway cell model.

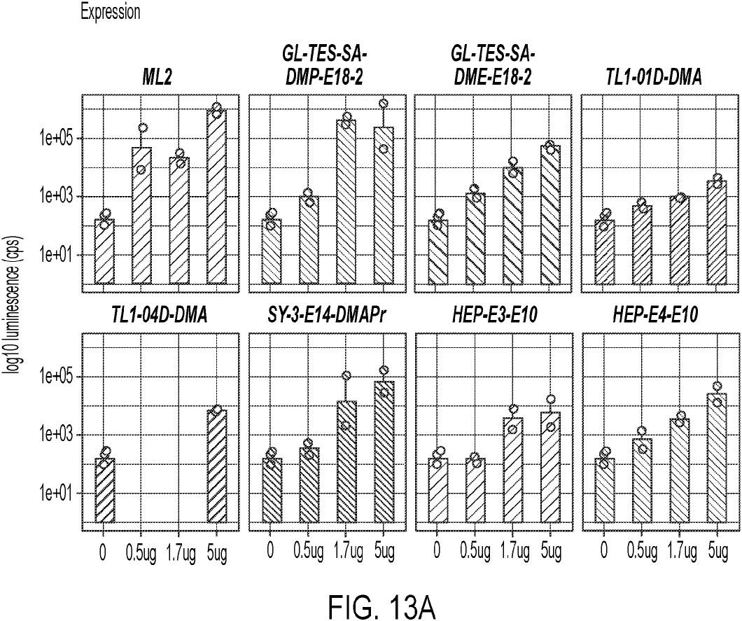

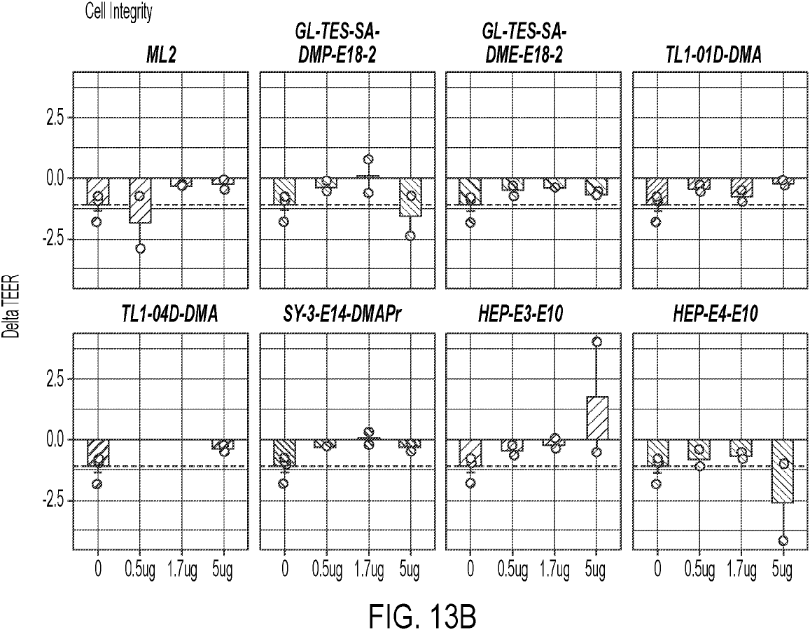

Cationic lipids ML2, GL-TES-SA-DMP-E18-2, GL-TES-SA-DME-E18-2, TL1-01D-DMA, TL1-04D-DMA, SY-3-E14-DMAPr, HEP-E3-E10, and HEP-E4-E10 were used to prepare LNPs encapsulating FFL mRNA. LNPs encapsulating FFL mRNA were added to apical layer of HBEC-ALI. Then, the luminescence was measured to evaluate the amount of luciferase protein expressed in the cells. As shown in FIG. 13A, all the mRNA-LNPs tested showed dose-dependent protein expression. Additionally, the mRNA-LNPs showed robust protein expression in the lung cells in the HEBC-ALI model. To examine if the human bronchial epithelial cells in the HBEC-ALI model maintain cell integrity during the experiments, trans-epithelial electrical resistance (TEER), which is a strong indicator of epithelium integrity, was measured. As shown in FIG. 13B, TEER did not significantly differ, indicating that the monolayer remained intact for most treatment with mRNA-LNPs. Therefore, the HBEC-ALI model can be used as a robust in vitro system for evaluating mRNA-LNPs for protein expression in lung cells.

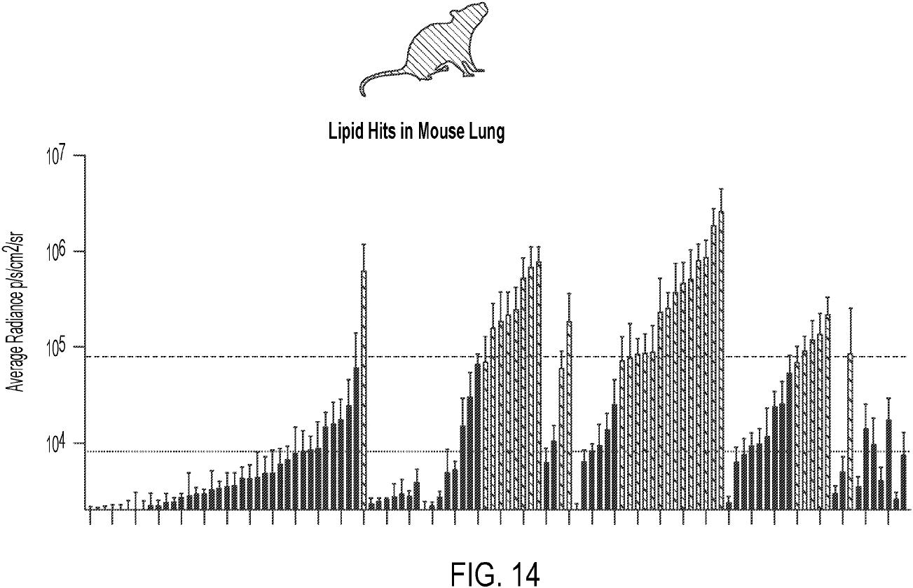

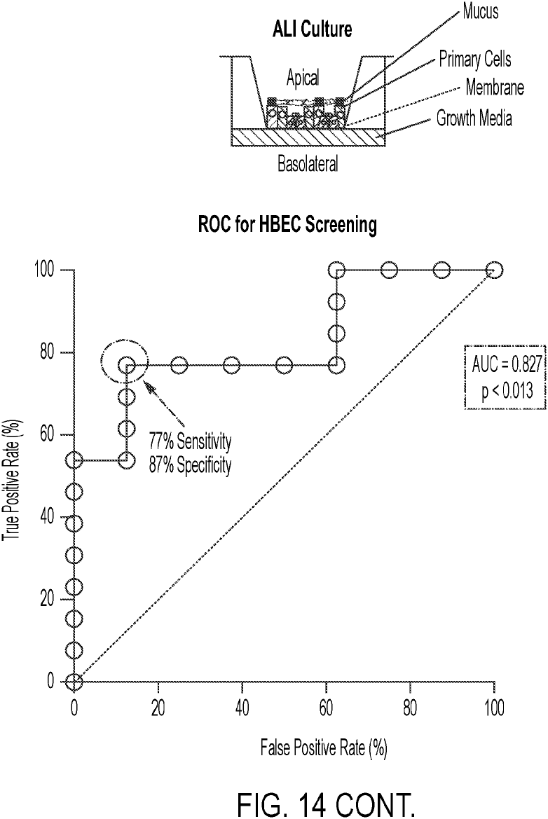

To further examine if the data from the HBEC-ALI model is a good predictor for in vivo protein expression, ROC curve (receiver operating characteristic curve) was plotted. Typically, the closer an ROC curve is to the upper left corner, the more efficient is the test. Statistics from the ROC curve show that the AUC (Area under the ROC Curve) is high (0.827) with a low p value (<0.013) (FIG. 14), indicating that data from HBEC-ALI model are translatable for determining in vivo potency of mRNA-LNPs, and that HBEC-ALI model can be used to predict mRNA-LNPs that warrant further investigation for in vivo applications.

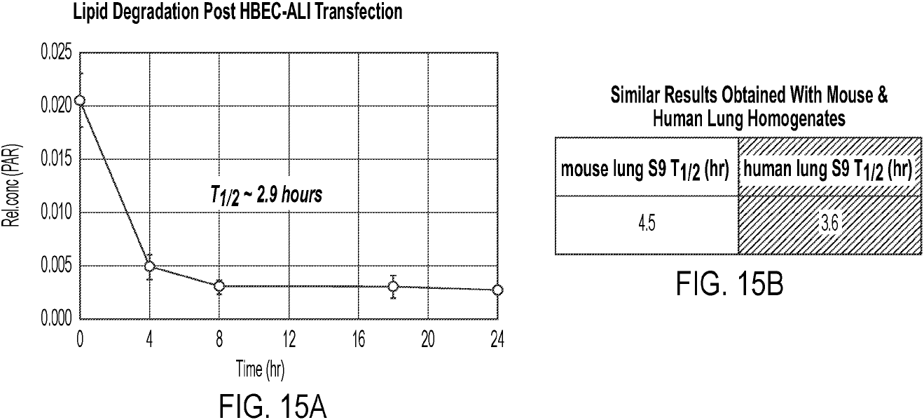

Next, lipid degradation rate post HBEC-ALI transfection was determined and compared to results obtained with mouse and human lung homogenates. It is desirable if the lipids degrade rapidly to reduce potential toxicity of LNP components, including cationic lipids. Concentration of lipids was measured in the HBEC-ALI sample culture over time and plotted as shown in FIG. 15A. The results show that after transfection with mRNA-LNP, the lipids degrade rapidly over time, with a half-life of about 2.9 hours. The half-life value determined from the HBEC-ALI model was comparable to values determined from mouse and human lung homogenates, which were 4.5 and 3.6 hours, respectively, as shown in FIG. 15B. These results demonstrate that the HBEC-ALI model is a useful indicator for the performance of mRNA-LNPs in vivo.

Overall, the data in this example demonstrate that HBEC-ALI shows meaningful performance as classification model for screening and filtering lipids prior to in vivo evaluation. Furthermore, the mRNA-LNPs of the present invention have robust protein expression and rapid degradation. Combined with the in vivo data presented herein, the mRNA-LNPs of the present invention are predicted to perform exceptionally well in terms of both increased potency and improved tolerability in in vivo application involving the repeat delivery of mRNA to the lung via nebulization.

### Example 9. Nebulized Delivery of Construct A

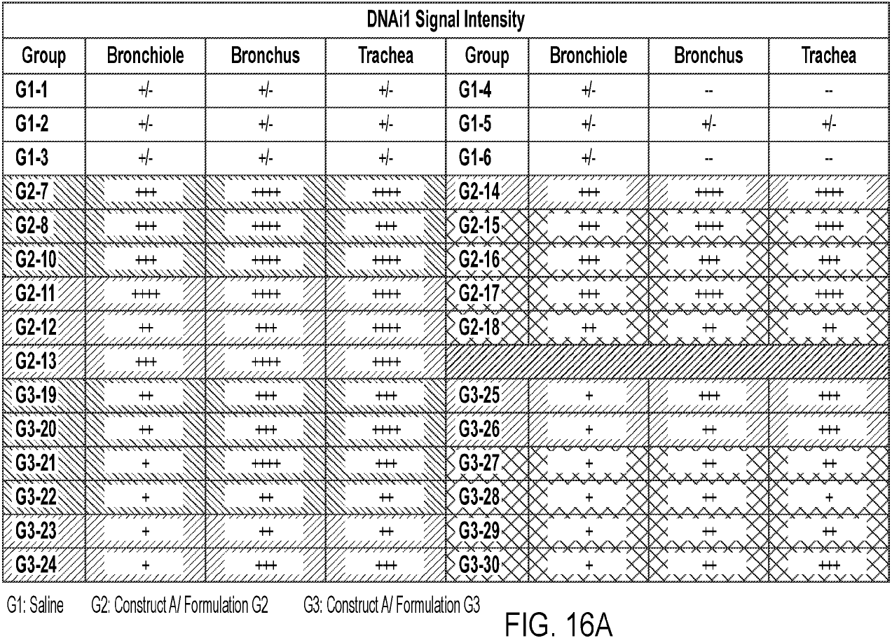

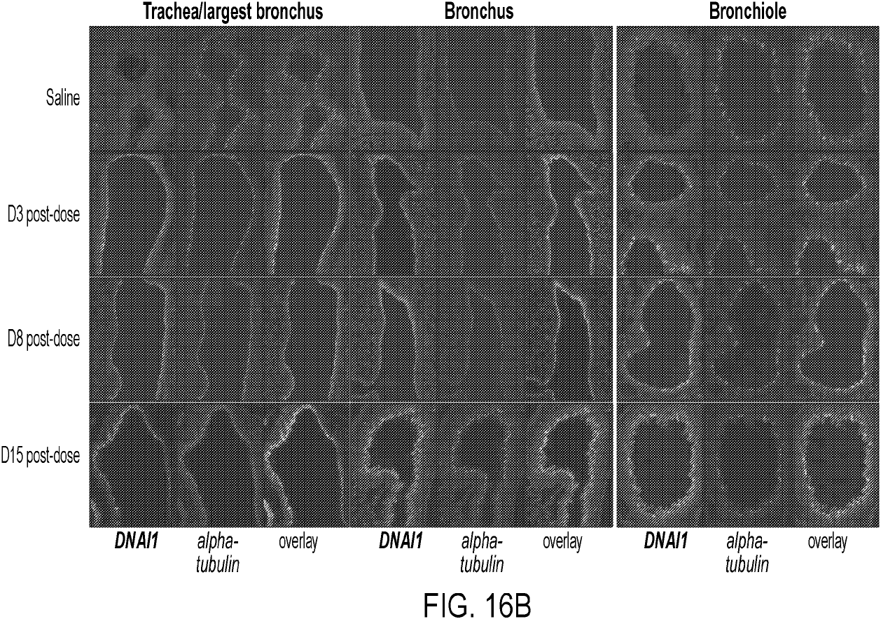

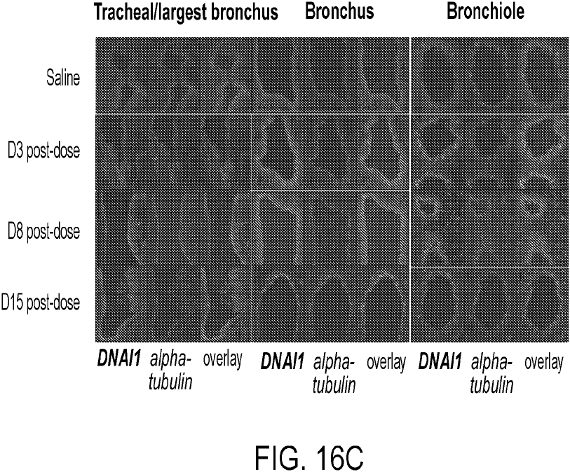

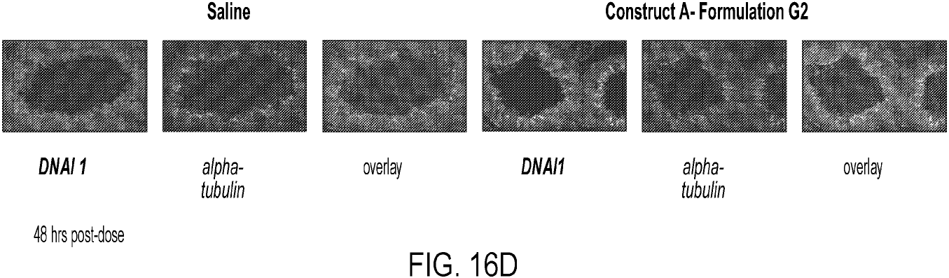

A pharmacodynamics (PD) study was performed in mice that were administered Construct A via nebulization. In this study, bio-distribution of DNA1I was determined after nebulized delivery of DNAI1. The study design is outlined in the Table 7 below. Construct A was delivered in one of two different lipid nanoparticles referred to as G2 and G3, respectively in FIG. 16A, and each comprising a different cationic lipid but the same helper lipid, a PEG-lipid and cholesterol.

TABLE 7

Delivery of Construct A via Nebulization Study Design

| Group No. | No. of Animals | Test Article | Dose Level (mg) | Conc. (mg/mL) | Dose Rate (ml/hr) | Dosing Regimen | Endpoint |
|---|---|---|---|---|---|---|---|
| 1 | 6 | Saline | 0 | 0 | NA | For 6 hours via aerosol inhalation using nebulizer | Harvest at 48 hr, d8, and d15 (n=4/time point |
| 2 | 12 | Construct A | 30 | 0.5 | 10 | | |
| 3 | 12 | Construct A | 30 | 0.5 | 10 | | |

The data acquired from these studies included double immunofluorescence for DNAI1 and alpha-tubulin from lungs, and proteonomics assessment. FIG. 16A shows a table summarizing immunofluorescence results acquired following administration via nebulization into wild type mice of Construct A in either formulation (G2 or G3). As shown in FIGS. 16A-D, fluorescence detection of DNAI1 delivered in either formulation via nebulization was present in bronchioles, trachea and bronchi. FIG. 16B shows confocal immunofluorescence images showing DNAI1 and α-tubulin staining of mouse lungs specimens obtained from mice which had received Construct A in Formulation G2. FIG. 16C shows confocal immunofluorescence images showing DNAI1 and α-tubulin staining of mouse lungs specimens obtained from mice which had received Construct A in Formulation G3. FIG. 16D shows a higher magnification confocal immunofluorescence image of mouse lungs from mice which had received Construct A in Formulation G2. With the immunofluorescence data showing colocalization of hDNAIl and α-tubulin in the bronchioles of wild type mice suggesting deep delivery of Construct A in both formulations tested, these data indicate that nebulization is a means to efficiently deliver mRNA comprising liposomes deep into lung tissue.

### Example 10. Repeat Dosing of Construct A or C in In Vivo Mouse Model

The example shows that repeat dosing results in increased and sustained expression of DNAI1 in anatomically appropriate tissues. A summary of the design of this study, including dosing interval is presented in Table 8 below.

TABLE 8

Repeat In Vivo Dosing Study Design

| Grps | # Animals/grp | Treatment | Dose Level ug/An | Conc (mg/mL) | Dose Volume | Dosing Regimen | Terminal time point |
|---|---|---|---|---|---|---|---|
| 1 | 3 | Saline | 0 | 0 | 50 | | |
| 2 | 24 | Construct A in Formulation G3 | 15 | 0.3 | 50 | Intratrcheal by Catheter on day 1, day 8, day 15, day 22, day 29, and day 36 | At d8, d15, d22, d29, d36, d38, d43, d50 (n=3) |
| 3 | 24 | Construct C in Formulation G3 | 15 | 0.3 | 50 | | |

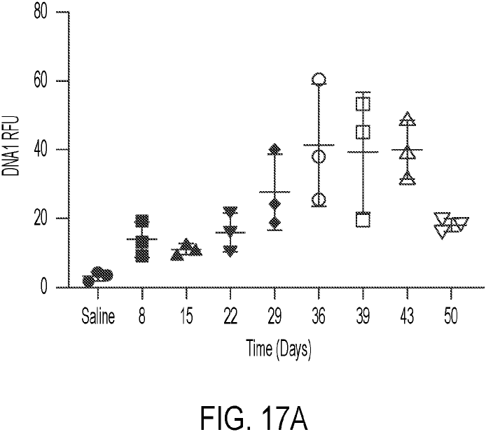

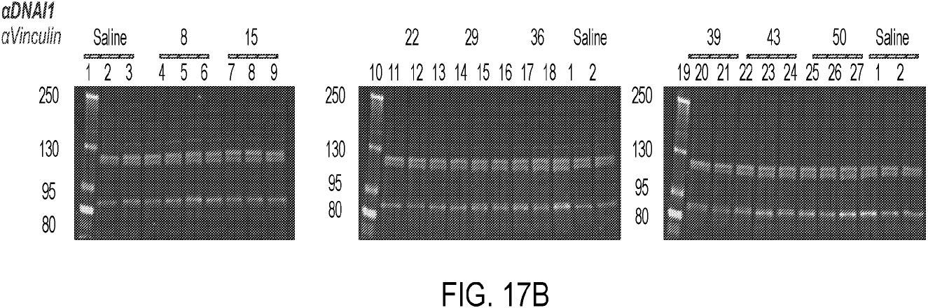

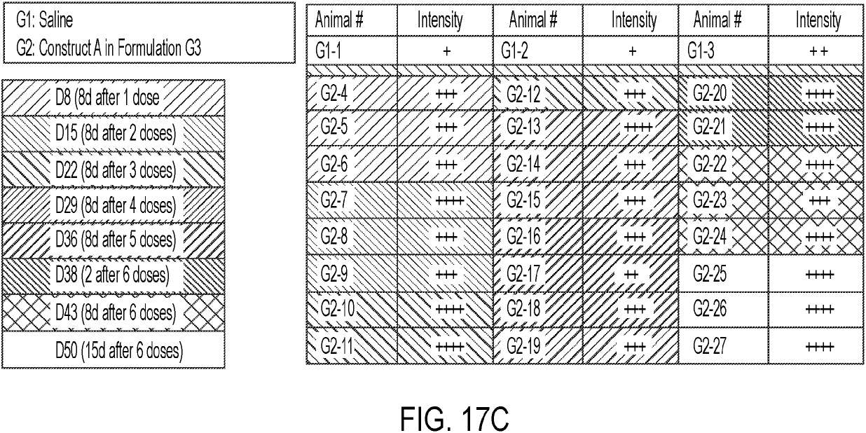

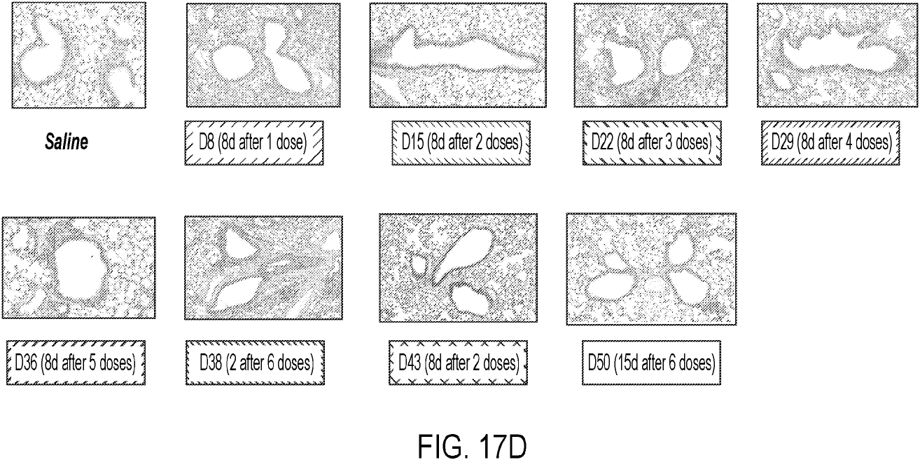

The data from these studies show that repeat dosing of Construct A resulted in progressively increased expression of DNA1 (FIG. 17A). Western blots of lung tissue isolates further supported that repeat dosing maintained high DNAI1 expression (FIG. 17B). Immunofluorescence studies showed that repeat dosing also resulted in high expression DNA1 (FIG. 17C). Appropriate anatomical localization of delivered Construct A was also observed in these studies (FIG. 17D). The data from the histological images in FIG. 17D show that repeat weekly dosing of hDNAI1 resulted in an increase in the amount of multi-ciliated cells associated with DNAI1. Moreover, DNAI1 protein expression was detectable by Western blot (FIG. 17B) and its biodistribution showed localized expression in bronchiolar multi-ciliated cells. It also was observed that the expressed human DNAI1 colocalized with alpha-Tubulin by immunofluorescence.

Collectively, these data showed (i) robust delivery of an mRNA-based therapeutic encoding DNAI1 and (ii) correct localization of wild-type protein strongly supporting use of mRNA therapeutics for the treatment of lung-associated diseases, such as PCD.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A method of delivery of a dynein axonemal intermediate chain 1 (DNAI1) protein, comprising administering to a subject in need of delivery an mRNA encoding the DNAI protein.

2. A method of treating primary ciliary dyskinesia (PCD) comprising administering to a subject in need of treatment an mRNA encoding a dynein axonemal intermediate chain 1 (DNAI1) at an effective dose and an administration interval such that at least one symptom or feature of PCD is reduced in intensity, severity, or frequency or has delayed in onset.

3. The method of claim 1 or claim 2, wherein the DNAI1 mRNA is encapsulated in a liposome.

4. The method of claim 3, wherein the liposome comprises one or more cationic lipids, one or more non-cationic lipids and one or more PEG-modified lipids.

5. The method of claim 4, wherein the one or more cationic lipids are selected from the group consisting of TL1-01D-DMA, TL1-10D-DMA, GL-TES-SA-DMP-E18-2, HEP-E4-E10, HEP-E3-E10 and TL1-04D-DMA, GL-TES-SA-DME-E18-2, Guan-SS-Chol, SY-3E-14-DMAPr, RL3-07D-DMA, cKK-E12, OF-02, ICE (Imidazol-based ester) and combinations thereof.

6. The method of claim 5, wherein the cationic lipid is TL1-04D-DMA.

7. The method of any one of the preceding claims , wherein the one or more non-cationic lipids are selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DOPC (1,2-dioleyl- sn-glycero-3 -pho sphotidylcholine) DPPE (1,2-dipalmitoyl- sn-glycero-3-phosphoethanolamine), DMPE (1,2-dimyristoyl-sn-glycero-3-phosphoethanolamine), DOPG (1,2-dioleoyl-sn-glycero-3-phospho-(1′-rac-glycerol)) or combinations thereof.

8. The method of any one of claims 4-7, wherein the one or more PEG-modified lipids comprise a poly(ethylene) glycol chain of up to 5 kDa in length covalently attached to a lipid with alkyl chain(s) of C₆-C₂₀ length.

9. The method of any one of the preceding claims , wherein the cationic lipid constitutes about 30-60 % of the liposome by molar ratio.

10. The method of claim 9, wherein the cationic lipid constitutes about 30%, 40 %, 50%, or 60% of the liposome by molar ratio.

11. The method of any one of claims 4-10, wherein the liposome comprises Guan-SS-Chol as the cationic lipid.

12. The method of claim 11, wherein the liposome comprises no more than three distinct lipid components.

13. The method of claim 12, wherein the three distinct lipid components are Guan-SS-Chol, DOPE and DMG-PEG2K.

14. The method of claim 13, wherein Guan-SS-Chol, DOPE and DMG-PEG2K are present in a molar ratio of approximately 60:35:5, respectively.

15. The method of any one of claims 4-10, wherein the liposome comprises four distinct lipid components.

16. The method of claim 15, wherein the four distinct lipid components are a cationic lipid, a non-cationic lipid, cholesterol and a PEG-modified lipid.

17. The method of claim 16, wherein the cationic lipid is selected from GL-TES-SA-DME-E18-2, TL1-01D-DMA, SY-3-E14-DMAPr, TL1-10D-DMA, GL-TES-SA-DMP-E18-2, HEP-E4-E10, HEP-E3-E10 and TL1-04D-DMA.

18. The method of claim 16 or 17, wherein the non-cationic lipid is DOPE or DEPE.

19. The method of claim 18, wherein the non-cationic lipid is DOPE.

20. The method of any one of claims 16-19, wherein the PEG-modified lipid is DMG-PEG2K.

21. The method of any one of claims 9-20, wherein the liposome comprises a molar ratio of cationic lipid to non-cationic lipid to cholesterol to PEG-modified lipid is between about 30-60:25-35:20-30:1-15, respectively.

22. The method of any one of claims 3-21, wherein the liposome has a diameter of about 80 nm to 200 nm, optionally wherein the liposome has a diameter of about 100 nm or less than 100 nm.

23. The method of any one of the preceding claims , wherein the DNAI1 mRNA is codon optimized.

24. The method of claim 23, wherein the codon-optimized mRNA produces at least 10% more, 15% more, 20% more, 25% more, or at least 30% more DNAI1 protein in comparison to a non-codon-optimized mRNA sequence.

25. The method of claim 24, wherein the codon-optimized mRNA produces at least 30% more DNAI1 protein in comparison to a non-codon-optimized mRNA sequence.

26. The method of any one of the preceding claims , wherein the DNAI1 mRNA comprises one or more modified nucleotides.

27. The method of claim 26, wherein the one or more modified nucleotides are selected from pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and/or 2-thiocytidine.

28. The method of any one of the preceding claims , wherein the mRNA is unmodified.

29. The method of any one of the preceding claims , wherein the mRNA comprises a 5′-untranslated region (5′-UTR) that has a sequence set forth in SEQ ID NO: 2 or 3.

30. The method of any one of the preceding claims , wherein the mRNA comprises a 3′-untranslated region (3′-UTR) that has a sequence set forth in SEQ ID NO: 4 or 5.

31. The method of any one of the preceding claims , wherein the mRNA comprises a coding sequence at least 70%, 75%, 80%, 85%, 90%, or 95% identical to any one of SEQ ID NO: 6 to SEQ ID NO: 10.

32. The method of any one of the preceding claims , wherein the mRNA comprises a coding sequence at least 80% identical to SEQ ID NO: 6 to SEQ ID NO: 10.

33. The method of any one of the preceding claims , wherein the mRNA comprises a coding sequence at least 90% identical to SEQ ID NO: 6 to SEQ ID NO: 10.

34. The method of any one of the preceding claims , wherein the mRNA comprises a coding sequence at least 95%, at least 96%, at least 97%, at least 98%, or at least 99% identical to SEQ ID NO: 6 to SEQ ID NO: 10.

35. The method of any one of the preceding claims , wherein the mRNA comprises a coding sequence set forth in SEQ ID NO: 6 to SEQ ID NO: 10.

36. The method of any one of the preceding claims , wherein administering the mRNA to the subject is performed by pulmonary, intratracheal, intranasal, intravenous, intramuscular or subcutaneous delivery.

37. The method of claim 36, wherein administering the mRNA to the subject is performed by pulmonary delivery.

38. The method of any one of the preceding claims , wherein administering the mRNA to the subject is performed by nebulization.

39. The method of any one of the preceding claims , wherein the composition is administered once a daily.

40. The method of any one of the preceding claims , wherein the composition is administered once a week.

41. The method of any one of the preceding claims , wherein the composition is administered once every two weeks.

42. The method of any one of the preceding claims , wherein the composition is administered twice a month.

43. The method of any one of the preceding claims , wherein the composition is administered once a month.

44. The method of any one of claims 36-38, wherein the composition is administered at repeat intervals.

45. The method of claim 44, wherein the repeat intervals occur every 3 days, 1 week, 2 weeks, 3 weeks, or four weeks.

46. The method of claim 45, wherein the repeat intervals occur every 4 weeks.

47. The method of any one of the preceding claims , wherein the administering the mRNA results in DNAI1 protein expression detectable in one or more internal organs selected from lung, heart, liver, spleen, kidney, brain, stomach, intestines, ovary and testis.

48. The method of claim 47, wherein the DNAI1 protein expression is detectable for at least 24 hours, 48 hours, 72 hours, 8 days or 15 days.

49. The method of any one of the preceding claims , wherein the administering the mRNA results in DNAI1 protein expression detectable in the lung.

50. The method of any one of the preceding claims , wherein the administering the mRNA results in DNAI1 protein expression detectable in the lung epithelium.

51. The method of any one of any one of preceding claims , wherein the DNAI1 protein expression is detectable throughout the length of cilia.

52. A composition for use in the treatment of primary ciliary dyskinesia (PCD), the composition comprising an mRNA encoding dynein axonemal intermediate chain 1 (DNAI1) encapsulated in a liposome, wherein the liposome comprises one or more cationic lipids, one or more non-cationic lipids and one or more PEG-modified lipids.

53. The composition of claim 52, wherein the mRNA comprises a coding sequence at least 70%, 75%, 80%, 85%, 90%, or 95% identical to any one of SEQ ID NO: 6 to SEQ ID NO: 10.

54. The composition of claim 52 or 53, wherein the mRNA comprises a coding sequence at least 70%, at least 80%, at least 90%, at least 95% or at least 98% identical to SEQ ID NO: 6 to SEQ ID NO: 10.

55. The composition of any one of claims 52-54, wherein the mRNA comprises a coding sequence set forth in SEQ ID NO: 6 to SEQ ID NO: 10.

56. The composition of any one of claims 52-55, wherein the mRNA has a 5′-untranslated region (5′-UTR) that has a sequence set forth in SEQ ID NO:2, and a 3′-untranslated region (3′-UTR) that has a sequence set forth in SEQ ID NO:4 or SEQ ID NO: 5.

57. The composition of any one of claims 52-56, wherein the mRNA has one or more modified nucleotides.

58. The composition of claim 57, wherein the modified one or more nucleotides is selected from pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and/or 2-thiocytidine.

59. The composition of any one of claims 52-56, wherein the mRNA is unmodified.

60. The composition of any one of claims 52-59, wherein the liposome is 150 nm in diameter or less.

61. The composition of any one of claims 52-60, wherein the one or more cationic lipids are selected from the group consisting of TL1-01D-DMA, TL1-10D-DMA, GL-TES-SA-DMP-E18-2, HEP-E4-E10, HEP-E3-E10, TL1-04D-DMA, GL-TES-SA-DME-E18-2, Guan-SS-Chol, SY-3-E14-DMAPr, RL3-07D-DMA, cKK-E12, OF-02, ICE (Imidazol-based ester)and combinations thereof.

62. The composition of any one of claims 52-61, wherein the cationic lipid is ICE.

63. The composition of any one of claims 52-61, wherein the cationic lipid is Guan-SS-Chol.

64. The composition of any one of claims 52-63, wherein the one or more non-cationic lipids are selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DOPC (1,2-dioleyl- sn-glycero-3 -pho sphotidylcholine) DPPE (1,2-dipalmitoyl- sn-glycero-3-phosphoethanolamine), DMPE (1,2-dimyristoyl-sn-glycero-3-phosphoethanolamine), DOPG (1,2-dioleoyl-sn-glycero-3-phospho-(1′-rac-glycerol)) or combinations thereof.

65. The composition of any one of claims 52-64, wherein the non-cationic lipid is DOPE.

66. The composition of any one of claims 52-65, wherein the one or more PEG-modified lipids comprise a poly(ethylene) glycol chain of up to 5 kDa in length covalently attached to a lipid with alkyl chain(s) of C₆-C₂₀ length.

67. The composition of claim 66, wherein the PEG-modified lipid is DMG-PEG2K.

68. The composition of any one of claims 52-67, wherein the liposome comprises no more than three distinct lipid components.

69. The composition of claim 68, wherein the three distinct lipid components are ICE, DOPE and DMG-PEG2K, optionally in a molar ratio of approximately 60:35:5, respectively.

70. The composition of claim 68, wherein the three distinct lipid components are Guan-SS-Chol, DOPE and DMG-PEG2K, optionally in a molar ratio of approximately 60:35:5, respectively.

71. The composition of any one of claims 52-67, wherein the liposome comprises four distinct lipid components.

72. The composition of claim 71, wherein the four distinct lipid components are a cationic lipid, a non-cationic lipid, cholesterol and a PEG-modified lipid.

73. The composition of claim 72, wherein the cationic lipid is selected from GL-TES-SA-DME-E18-2, TL1-01D-DMA, SY-3-E14-DMAPr, TL1-10D-DMA, GL-TES-SA-DMP-E18-2, HEP-E4-E10, HEP-E3-E10 and TL1-04D-DMA.

74. The composition of claim 72 or 73, wherein the non-cationic lipid is DOPE.

75. The composition of any one of claims 73-74, wherein the PEG-modified lipid is DM-PEG2K.

76. The composition of any one of claims 73-75, wherein the molar ratio of cationic lipid to non-cationic lipid to cholesterol to PEG-modified lipid is between about 30-60:25-35:20-30:1-15, respectively.

77. A pharmaceutical composition comprising the composition of any one of claims 52-76 and a suitable excipient.


SEQUENCE LISTING (16 sequences)

SEQ ID NO: 1 - protein, 699 aa, Homo sapiens
MIPASAKAPHKQPHKQSISIGRGTRKRDEDSGTEVGEGTDEWAQSKATVRPPDQLELTDAELKEEFTRILTANNPHAPQNIVRYSFKEGTYKPIGFVNQLAVHYTQVGNLIPKDSDEGRRQHYRDELVAGSQESVKVISETGNLEEDEEPKELETEPGSQTDVPAAGAAEKVTEEELMTPKQPKERKLTNQFNFSERASQTYNNPVRDRECQTEPPPRTNFSATANQWEIYDAYVEELEKQEKTKEKEKAKTPVAKKSGKMAMRKLTSMESQTDDLIKLSQAAKIMERMVNQNTYDDIAQDFKYYDDAADEYRDQVGTLLPLWKFQNDKAKRLSVTALCWNPKYRDLFAVGYGSYDFMKQSRGMLLLYSLKNPSFPEYMFSSNSGVMCLDIHVDHPYLVAVGHYDGNVAIYNLKKPHSQPSFCSSAKSGKHSDPVWQVKWQKDDMDQNLNFFSVSSDGRIVSWTLVKRKLVHIDVIKLKVEGSTTEVPEGLQLHPVGCGTAFDFHKEIDYMFLVGTEEGKIYKCSKSYSSQFLDTYDAHNMSVDTVSWNPYHTKVFMSCSSDWTVKIWDHTIKTPMFIYDLNSAVGDVAWAPYSSTVFAAVTTDGKAHIFDLAINKYEAICNQPVAAKKNRLTHVQFNLIHPIIIVGDDRGHIISLKLSPNLRKMPKEKKGQEVQKGPAVEIAKLDKLLNLVREVKIKT

SEQ ID NO: 2 - RNA, 140 nt, Artificial Sequence
  Synthetic Polynucleotide
agacagaucgccuggagacgccauccacgcuguuuugaccuccauagaagacaccgggaccgauccagccuccgcggccgggaacggugcauuggaacgcggauuccccgugccaagagugacucaccguccuugacacg

SEQ ID NO: 3 - RNA, 140 nt, Artificial Sequence
  Synthetic Polynucleotide
ggacagaucgccuggagacgccauccacgcuguuuugaccuccauagaagacaccgggaccgauccagccuccgcggccgggaacggugcauuggaacgcggauuccccgugccaagagugacucaccguccuugacacg

SEQ ID NO: 4 - RNA, 105 nt, Artificial Sequence
  Synthetic Polynucleotide
cggguggcaucccugugaccccuccccagugccucuccuggcccuggaaguugccacuccagugcccaccagccuuguccuaauaaaauuaaguugcaucaagcu

SEQ ID NO: 5 - RNA, 105 nt, Artificial Sequence
  Synthetic Polynucleotide
ggguggcaucccugugaccccuccccagugccucuccuggcccuggaaguugccacuccagugcccaccagccuuguccuaauaaaauuaaguugcaucaaagcu

SEQ ID NO: 6 - DNA, 2100 bp, Artificial Sequence
  Synthetic Polynucleotide
atgatccccgcctccgccaaagcccctcacaagcaaccgcacaagcaaagcattagcattgggcggggtactcggaagcgcgacgaggactcgggaactgaagtcggagaggggaccgacgaatgggcgcagtcaaaggccaccgtgcgcccaccggaccagctcgagctgaccgatgctgagctgaaggaggagtttacccggatcctgacagccaacaacccacatgcaccgcagaacatcgtgcggtacagcttcaaagagggaacttataagcccattggcttcgtgaaccaactcgcggtgcattacacccaagtcggaaaccttattccgaaggactcggacgaaggcagacgccagcactaccgggacgagctcgtggcaggatcccaggaaagcgtcaaggtcatttccgagactggcaacctcgaggaggacgaagaacctaaggagctggaaaccgaacccggatcccagaccgacgtgccggccgctggggctgccgagaaagtcactgaagaggaactcatgaccccgaagcagccgaaagagagaaagctcaccaaccaattcaacttcagcgagcgcgccagccaaacctacaacaacccagtcagggatcgggaatgtcagaccgaaccgcctccgagaacgaacttctcggcgaccgcgaaccaatgggagatctacgacgcctacgtggaagaactggaaaagcaggaaaagactaaggaaaaggaaaaggccaagactcccgtcgccaagaagtcgggcaaaatggccatgcggaagctcacctccatggaatcacagactgacgacttgatcaagttgagccaggccgcaaagatcatggagcgcatggtcaaccaaaatacttacgacgatatcgcccaagacttcaagtactacgacgacgctgccgatgaataccgagatcaagtcggcaccctactgccgctttggaagttccagaatgacaaggccaagaggctgagcgtgaccgcgctgtgctggaaccccaaataccgcgacctcttcgccgtgggatacggctcctacgatttcatgaagcagagccggggaatgttgctcctttactccctgaagaacccctccttccctgagtacatgttcagctcaaacagcggcgtgatgtgcctcgacattcacgtggaccacccttacctcgtggccgtgggtcactacgacggcaacgtcgcgatctacaacttgaagaagccgcattcacagccctcgttttgctcctcggccaagtccggcaaacattcggacccagtgtggcaagtcaagtggcagaaagatgacatggaccaaaacttgaacttcttcagcgtgtcctccgacggacggatcgtgtcctggaccctcgtgaagcggaagttggtgcatatcgacgtgatcaaattgaaggtcgagggttcgaccaccgaagtgcctgaaggcctgcagcttcaccccgtgggatgcggcactgccttcgacttccacaaggagatcgactacatgttcctcgtgggaaccgaggaagggaagatctacaaatgcagcaagtcctactcatcacaattcctggatacctacgatgcccacaacatgagcgtggataccgtgtcgtggaacccctatcacaccaaggtattcatgtcctgctcctccgactggaccgtcaagatttgggaccacaccatcaagacccccatgttcatctacgacctgaactccgccgtgggggatgtggcctgggccccctactcgtcgaccgtgtttgccgcggtcaccacggacggaaaggcacacattttcgaccttgcgattaacaaatacgaggcgatttgcaaccagcccgtggccgccaaaaagaaccgcctgacccacgttcaattcaacttaatccacccaatcatcatcgtcggcgatgacagaggacacattattagcctgaaacttagccccaacctccgcaagatgcccaaggagaagaagggacaggaagtccagaagggccctgccgtggagattgcaaagctcgataagctcctgaacttagtccgggaagtgaagatcaagacttaa

SEQ ID NO: 7 - DNA, 2100 bp, Artificial Sequence
  Synthetic Polynucleotide
atgatccccgctagcgctaaggcccctcacaaacagccccacaagcagagcattagcattggacgcggcacacggaaacgggacgaagattccggaaccgaagtcggggaaggcaccgatgaatgggcccagtccaaggccaccgtgcggcctcctgaccaattggaactgactgacgcggaactgaaggaagaattcaccagaattctaaccgccaacaatcctcatgccccgcaaaacatcgtgcgctactcattcaaggaaggaacctacaagcccattggattcgtgaaccaactcgccgtgcactacactcaagtcggcaacttaattcccaaggattcggacgagggccgccggcagcactacagggatgagcttgtggcaggctcacaggaaagcgtcaaagtcattagcgaaaccggcaaccttgaagaggatgaggagcctaaggagctggaaaccgagccgggttcgcaaaccgacgtgcctgccgccggagccgcagagaaggtcaccgaagaggaactcatgacccccaagcagccgaaggagcgcaaactcactaaccaattcaacttcagcgagagagcgagccagacctacaacaacccagtgcgcgaccgggaatgccagactgagccgcccccgagaactaacttcagcgcgactgccaaccaatgggagatctacgacgcctacgtggaagagttggagaagcaggaaaagaccaaggagaaggaaaaggccaagaccccggtggcgaagaagtccggaaagatggcgatgcggaagttgacgtccatggaatcccagaccgatgacctcatcaagctcagccaggcagcaaagatcatggaaaggatggtgaaccagaacacctacgatgacattgctcaggactttaagtactacgacgatgccgccgacgaataccgcgaccaggtcggaaccctcctcccgctttggaaattccagaacgataaagccaagcgcctcagcgtgactgcgctgtgctggaacccgaagtaccgggaccttttcgccgtgggctatggatcctacgacttcatgaagcaatcgcgggggatgctcctcctgtatagcctgaagaacccgagcttccccgagtacatgttcagctcaaactccggagtgatgtgcctcgacatccacgtggaccacccatacttggtggccgtgggccactacgatggcaatgtcgcgatctacaacctcaagaagccccactcccaaccctcgttctgcagcagcgccaagagcgggaaacactccgacccagtgtggcaagtcaaatggcagaaggacgacatggatcagaacttgaacttcttcagcgtgtcctcagacggacggatcgtgtcctggaccctcgtgaagaggaagttggtccatatcgacgtgatcaagctcaaggtcgagggttcaaccaccgaggtgcccgagggcctgcagctccaccccgtgggctgcggcacggccttcgactttcataaggaaattgactacatgttcctcgtggggactgaagagggaaagatctacaagtgcagcaaatcctactcgtcccaattcctcgacacctacgatgcgcacaacatgagcgtcgacacggtgtcatggaacccctaccatacaaaggtattcatgtcgtgctcctccgactggaccgtgaagatttgggatcacactatcaagaccccgatgttcatatacgacctgaactccgccgtgggagatgtggcttgggcgccctactcatcgaccgtgtttgccgccgtgaccaccgacggaaaggctcacattttcgatcttgccatcaacaagtacgaggcgatttgcaaccagccagtggccgccaagaagaacagactgactcacgtgcagtttaacttaatccaccccattattatcgtgggggacgaccgcggacacattattagcctgaagctcagcccgaacctccggaagatgcctaaggagaagaaggggcaggaagtgcagaaaggcccggccgtggaaattgccaaattggacaagttactgaaccttgtccgcgaagtcaagatcaaaacttaa

SEQ ID NO: 8 - DNA, 2100 bp, Artificial Sequence
  Synthetic Polynucleotide
atgatccccgcatccgccaaagcccctcataaacagccccacaaacagtccatctccattggacgggggacccggaaaagggatgaggactctgggacggaagttggagaaggcactgacgaatgggcacagagtaaggctaccgtgagacctcccgaccagctggagctcactgacgcagaactgaaggaggagtttactaggatcctgacagcaaataacccccacgccccacagaatatcgtcagatatagcttcaaagagggcacatacaagcctattgggttcgtgaaccagctggctgtgcattacacacaggtggggaaccttattcctaaagactctgatgaaggccgcagacagcattatagagatgaactggttgcaggatcccaagagtctgtgaaagtgattagcgagaccggcaacctggaagaagatgaggaaccaaaagaactggagacagagcctgggtctcagacagacgtgccagcagctggcgctgccgagaaagtgacagaggaggagctgatgacacctaaacagccaaaagagaggaagctgacaaaccaattcaatttttccgaacgggcatcacagacctacaacaacccagtgcgcgaccgggagtgtcaaaccgaacctcctcctagaacaaacttttctgctactgcaaatcagtgggagatctacgatgcctacgtggaggagctggagaagcaggaaaagactaaggagaaggagaaggcaaagacccccgtggccaaaaaatccggcaaaatggcaatgcggaagctgacttctatggaaagccagactgatgacctgatcaaactgtcccaggcagctaagattatggaaaggatggtcaatcagaatacatatgacgacattgctcaggactttaagtattatgatgatgccgctgacgagtatcgggaccaagtggggacactgctgccactgtggaagtttcaaaacgacaaggctaaaaggctgtccgtgacagcactctgctggaatcccaagtaccgggacctctttgccgtggggtacggatcttacgacttcatgaaacagtccagaggcatgctgctgctgtacagcttgaagaacccctcctttcccgagtacatgttcagctctaattctggagtgatgtgcctggacatccacgtggatcacccttacctcgtggccgttggacactatgacggcaatgtggccatctacaacctgaaaaaaccacactctcagccttccttttgtagctctgcaaagtccggaaagcattccgaccccgtgtggcaagtgaaatggcagaaagacgacatggaccagaatctgaacttcttctccgtctcttcagacggcagaatcgtctcatggactctggtcaaacggaagctggttcacatcgacgtgatcaaactcaaggtcgaaggatcgactactgaggtgccagaaggactgcagctgcacccagtgggatgtggaactgcatttgatttccataaagaaatcgactacatgtttctggtgggaactgaagaggggaagatctataagtgtagcaaatcctattctagccagtttctggatacatacgacgctcacaacatgtccgtggacactgtaagctggaacccctatcataccaaggtgttcatgtcctgcagctccgattggactgttaagatttgggatcacacaatcaagacccctatgtttatctacgatctgaactctgccgtgggggatgtggcctgggcaccatatagctccacagtcttcgcagctgtcactaccgatggaaaggcccacatttttgacctggctatcaacaaatacgaggccatctgcaatcagcctgtggcagcaaagaagaaccgcctgactcacgtgcaattcaacctgattcaccctatcatcattgttggggatgataggggccacattatttctctaaagctgtccccaaatctgcggaaaatgcccaaggagaagaaaggccaggaggtgcagaaaggcccagccgttgaaatcgcaaagctggacaagctgctcaacctcgtccgggaggttaaaatcaaaacctga

SEQ ID NO: 9 - DNA, 2100 bp, Artificial Sequence
  Synthetic Polynucleotide
atgatcccagcaagcgccaaggccccacacaaacagccccacaagcagtcgatcagcattggcagggggactcgcaagagagacgaggactccggaacagaagtgggggaggggacagatgaatgggcccagtctaaggccactgttcgccctccggatcagctggaactgacagatgccgagctgaaggaagagttcaccaggattctgactgcaaataatccacacgctccacagaacattgtgagatattcttttaaggagggcacttacaaacccatcgggtttgtgaatcagctggcagtgcattacactcaagtgggcaacctgatccccaaagactctgatgaagggaggcggcagcactatagggacgagctggtcgctgggtcccaagagagcgtgaaagtcatttctgagactggcaacctggaagaggatgaggagccaaaggagctggagactgaaccagggtctcagacagatgtgcccgccgctggagctgctgagaaggtgacagaggaggaactgatgacccctaaacagcctaaggaacggaagctcaccaaccagttcaacttcagcgaaagagctagccagacttataataaccctgtgcgcgaccgggagtgtcagactgagcccccaccaagaaccaatttctccgccactgccaaccagtgggaaatctatgacgcttacgtcgaggagctggagaaacaggagaaaactaaggagaaagaaaaggccaaaacacccgtcgccaaaaagtctggcaagatggccatgagaaaactgacctccatggagtctcagaccgacgacctgatcaaactgtcccaggcagccaagatcatggagaggatggtgaaccagaacacctatgatgacattgcccaggactttaaatactacgatgatgccgctgacgagtatcgggaccaggtggggactctgctgcctctgtggaaattccagaatgataaggctaaacgcctgtccgtgaccgccctctgctggaaccctaagtaccgcgacctctttgctgtggggtacggatcttacgacttcatgaaacagtccagaggcatgctgctgctgtacagcttgaagaacccctcctttcccgagtacatgttcagctctaattctggagtgatgtgcctggacatccacgtggatcacccttacctcgtggccgttggacactatgacggcaatgtggccatctacaacctgaaaaaaccacactctcagccttccttttgtagctctgcaaagtccggaaagcattccgaccccgtgtggcaagtgaaatggcagaaagacgacatggaccagaatctgaacttcttctccgtctcttcagacggcagaatcgtctcatggactctggtcaaacggaagctggttcacatcgacgtgatcaaactcaaggtcgaaggatcgactactgaggtgccagaaggactgcagctgcacccagtgggatgtggaactgcatttgatttccataaagaaatcgactacatgtttctggtgggaactgaagaggggaagatctataagtgtagcaaatcctattctagccagtttctggatacatacgacgctcacaacatgtccgtggacactgtaagctggaacccctatcataccaaggtgttcatgtcctgcagctccgattggactgttaagatttgggatcacacaatcaagacccctatgtttatctacgatctgaactctgccgtgggggatgtggcctgggcaccatatagctccacagtcttcgcagctgtcactaccgatggaaaggcccacatttttgacctggctatcaacaaatacgaggccatctgcaatcagcctgtggcagcaaagaagaaccgcctgactcacgtgcaattcaacctgattcaccctatcatcattgttggggatgataggggccacattatttctctaaagctgtccccaaatctgcggaaaatgcccaaggagaagaaaggccaggaggtgcagaaaggcccagccgttgaaatcgcaaagctggacaagctgctcaacctcgtccgggaggttaaaatcaaaacctga

SEQ ID NO: 10 - DNA, 2100 bp, Artificial Sequence
  Synthetic Polynucleotide
atgatcccagcttctgccaaggccccacacaagcagccacacaaacagagcatttccattgggcgcggcacaaggaagagagacgaggactcaggcacagaggtgggcgaaggaaccgacgagtgggctcagagcaaagccacagtgaggcccccagatcagctggagctgacagacgccgagctgaaggaggagtttacccgcatcctgactgccaataacccacacgcaccccagaacatcgtgcgctattcttttaaggaaggaacctataagccaatcggctttgtcaatcagctggctgtgcactacacccaggttgggaacctgatccccaaggatagcgacgagggcaggagacagcattatagagacgagctcgtcgccggaagccaggagtctgtcaaagtgatcagcgaaacaggaaacctggaggaggatgaggagcccaaggaactggaaaccgagcctggcagccagacagatgtgccagccgcaggagccgcagagaaggtgacagaagaggagctcatgacccccaaacagccaaaggagcggaaactgacaaaccagttcaacttcagcgaaagagccagccagacctacaataaccccgtgcgggacagagaatgccagacagagcctccaccacgcaccaacttctccgcaacagctaaccagtgggagatctatgatgcctacgtggaggagctggaaaagcaggagaagaccaaagaaaaggagaaagccaagacccctgtcgccaagaagtccggcaaaatggctatgagaaagctgacatctatggaatcccagactgatgacctgatcaagctgtctcaggcagccaagattatggaaagaatggtgaatcagaacacctatgacgacatcgcccaggattttaagtactatgatgacgctgcagacgagtatagagatcaggtggggaccctgctgccactgtggaagttccagaatgacaaggctaagcgcctgtccgtgacagctctgtgctggaatccaaaatatagggacctcttcgccgtgggctacggctcttatgacttcatgaagcagtcacgcgggatgctgctgctgtacagcctgaaaaatccctcctttcccgagtacatgttcagctctaactccggggtcatgtgtctggatattcatgtggaccatccatacctggtggctgtcgggcactacgatggaaacgtggctatctacaatctgaagaagccacactcccagccctccttttgctcctccgccaagtccggcaagcactccgaccctgtgtggcaggtcaagtggcagaaggacgacatggaccagaacctgaacttcttttctgtgtctagcgatggcaggatcgtgtcctggaccctggtgaagagaaaactggtgcacatcgatgttatcaagctcaaagtcgagggaagcaccaccgaggttcctgagggcctgcagctgcacccagtgggctgcggcacagccttcgactttcataaagagattgactacatgttcctggtgggcacagaggaggggaagatctacaagtgctccaaatcctactccagccagtttctggacacttacgacgctcataatatgagcgtggacaccgtgtcctggaacccttaccacacaaaggtgttcatgagctgcagcagcgactggactgtgaagatttgggaccatactatcaaaaccccaatgtttatctatgatctcaattctgccgtgggcgacgtggcttgggccccctattcctccacagtgttcgcagccgtgactaccgacggaaaagcccacattttcgacctcgctattaacaagtatgaggccatttgtaaccagccagtggctgccaagaagaaccgcctgacccacgtgcagttcaacctgattcacccaattatcattgtgggggacgacagaggacacattatctcactgaagctgtctcctaatctgagaaagatgcctaaggagaagaaaggacaggaggtgcagaagggccctgccgtggaaattgccaaactcgacaagctgctgaacctggtgagggaggtgaagatcaagacatga

SEQ ID NO: 11 - DNA, 2106 bp, Artificial Sequence
  Synthetic Polynucleotide
atgccatccaaacaaattagaaagcagagcattagcgtgacccgcggagcccggagaagagatgaggactcgggcaccgatgtcggcgaaggaaccgacgagtggtcgcagtcaaaggctaccgtgcggccgcctgaccagcttgaacttaccgatgccgagctgaaggaagagttcactcgcatcctgaccgccaacaatccccacgcgccgcagaacatcgtgcgctactccttcaaagaaggaacgtacaagctcattggattcgtgaaccaaatggccgtgcacttttcccaagtcggaaacctcattccgaaggattccgacgagggccggcggcagcattaccgcgacgaaatggtggccggatcgcaagaaagcatcaaggtcgtgacttccgaggccgagaacctcgaggaagaagaagaaccgaaagagggcgaaggagaagccgaagccgaggctgaggccggaagccagaccgatatccccgctgccgcagaaactaccgaaaaggtcatcgaagaggagttgatggccccggtgcagcctaaggaacggaagttgactaaccaattcaacttcagcgagagagcctcacagaccttcaacaacccactgagagatagggaatgtcaaatggagcctccgccccggacgaacttctcggccactgcaaaccaatgggagatctacgacgcttacgtggatgaactggagaagcaggaaaagaccaaggaaaaggagaaagccaagaccccggtcgccaaaaagactgagaaaatggccatgcggaagctcacttccatggagagccagtccgacgatattactaaggttacccaggccgccaagatcgtggaaagaatggtgaaccagaatacctacgacgacgtggcacaggattttaagtactatgaggacaccgctgacgaataccgggatcaagaaggcaccctcctgcccttgtggaagttccagaatgacaaggccaagaggctggccgtgactgcgctgtgctggaacccgaagtataaggacctcttcgccgtgggtcatggctcatacgactttatgaagcaatcgcgcgggatgctcctgctctactcgatgaagaacccttccttccccgagtacatgttcagctccgagtcagggatcatgtgcctggatgtccatgtcgaccacccttacctcgtggtcgtgggatattacgacggcaacgtggcgatctacaaccttaagaaaccgcatagccagccctgcttccggagcacctcaaagagcggaaagcacaccgaccccgtgtggcaagtcaagtggcaaaaggacgacatggaccacaacttgaacttcttcagcgtgtcgtccgacggacggatcgtgtcctggaccctcgtcaagtccgagctcgtgcacattgacattatcaagctcaaaaccgagggttccaccactgaaatccccgagggcctgcagctccataccgtgggatgcggcaccgcgttcgacttccataaagagatcgactacatgttcctcgtggggactgaagaggggaagatctacaagtgcagcaagtcgtactcttcccaattcctggacacctacgacgctcataacatggcagtggacgccgtgctgtggaacccataccacactaaggtctttatgtcctgctcctcggactggactgtgaagatttgggaccacaccatcaaaaccccaatgttcatttacgacctgaactccgccgtgggggatgtggcctgggccccgtattcctccaccgtattcgccgccgtgaccaccgacggaaaggcacacgtctttgacctggccgtgaacaaatacgaggcgatttgcaaccagccagtggtggccaagaagaaaaacaagatcactcacgtgcaattcaaccccatccaccccattattatcgtcggcgacgataggggtcacattatatgcctgaagctcagcccgaaccttcgcaagatgcctaaggaaaagaagggccaggaggtgcagaagggtcctgccgtcgaaattgcgaagttggacaagctcctgaacctcgtgcgcgaggtcaagactaagacctaa

SEQ ID NO: 12 - DNA, 2136 bp, Artificial Sequence
  Synthetic Polynucleotide
atgatccccgcctccgccaaagcccctcacaagcaaccgcacaagcaaagcattagcattgggcggggtactcggaagcgcgacgaggactcgggaactgaagtcggagaggggaccgacgaatgggcgcagtcaaaggccaccgtgcgcccaccggaccagctcgagctgaccgatgctgagctgaaggaggagtttacccggatcctgacagccaacaacccacatgcaccgcagaacatcgtgcggtacagcttcaaagagggaacttataagcccattggcttcgtgaaccaactcgcggtgcattacacccaagtcggaaaccttattccgaaggactcggacgaaggcagacgccagcactaccgggacgagctcgtggcaggatcccaggaaagcgtcaaggtcatttccgagactggcaacctcgaggaggacgaagaacctaaggagctggaaaccgaacccggatcccagaccgacgtgccggccgctggggctgccgagaaagtcactgaagaggaactcatgaccccgaagcagccgaaagagagaaagctcaccaaccaattcaacttcagcgagcgcgccagccaaacctacaacaacccagtcagggatcgggaatgtcagaccgaaccgcctccgagaacgaacttctcggcgaccgcgaaccaatgggagatctacgacgcctacgtggaagaactggaaaagcaggaaaagactaaggaaaaggaaaaggccaagactcccgtcgccaagaagtcgggcaaaatggccatgcggaagctcacctccatggaatcacagactgacgacttgatcaagttgagccaggccgcaaagatcatggagcgcatggtcaaccaaaatacttacgacgatatcgcccaagacttcaagtactacgacgacgctgccgatgaataccgagatcaagtcggcaccctactgccgctttggaagttccagaatgacaaggccaagaggctgagcgtgaccgcgctgtgctggaaccccaaataccgcgacctcttcgccgtgggatacggctcctacgatttcatgaagcagagccggggaatgttgctcctttactccctgaagaacccctccttccctgagtacatgttcagctcaaacagcggcgtgatgtgcctcgacattcacgtggaccacccttacctcgtggccgtgggtcactacgacggcaacgtcgcgatctacaacttgaagaagccgcattcacagccctcgttttgctcctcggccaagtccggcaaacattcggacccagtgtggcaagtcaagtggcagaaagatgacatggaccaaaacttgaacttcttcagcgtgtcctccgacggacggatcgtgtcctggaccctcgtgaagcggaagttggtgcatatcgacgtgatcaaattgaaggtcgagggttcgaccaccgaagtgcctgaaggcctgcagcttcaccccgtgggatgcggcactgccttcgacttccacaaggagatcgactacatgttcctcgtgggaaccgaggaagggaagatctacaaatgcagcaagtcctactcatcacaattcctggatacctacgatgcccacaacatgagcgtggataccgtgtcgtggaacccctatcacaccaaggtattcatgtcctgctcctccgactggaccgtcaagatttgggaccacaccatcaagacccccatgttcatctacgacctgaactccgccgtgggggatgtggcctgggccccctactcgtcgaccgtgtttgccgcggtcaccacggacggaaaggcacacattttcgaccttgcgattaacaaatacgaggcgatttgcaaccagcccgtggccgccaaaaagaaccgcctgacccacgttcaattcaacttaatccacccaatcatcatcgtcggcgatgacagaggacacattattagcctgaaacttagccccaacctccgcaagatgcccaaggagaagaagggacaggaagtccagaagggccctgccgtggagattgcaaagctcgataagctcctgaacttagtccgggaagtgaagatcaagactgggggcggatccgactataaggatgatgacgataagtaa

SEQ ID NO: 13 - DNA, 2139 bp, Artificial Sequence
  Synthetic Polynucleotide
atgatccccgcctccgccaaagcccctcacaagcaaccgcacaagcaaagcattagcattgggcggggtactcggaagcgcgacgaggactcgggaactgaagtcggagaggggaccgacgaatgggcgcagtcaaaggccaccgtgcgcccaccggaccagctcgagctgaccgatgctgagctgaaggaggagtttacccggatcctgacagccaacaacccacatgcaccgcagaacatcgtgcggtacagcttcaaagagggaacttataagcccattggcttcgtgaaccaactcgcggtgcattacacccaagtcggaaaccttattccgaaggactcggacgaaggcagacgccagcactaccgggacgagctcgtggcaggatcccaggaaagcgtcaaggtcatttccgagactggcaacctcgaggaggacgaagaacctaaggagctggaaaccgaacccggatcccagaccgacgtgccggccgctggggctgccgagaaagtcactgaagaggaactcatgaccccgaagcagccgaaagagagaaagctcaccaaccaattcaacttcagcgagcgcgccagccaaacctacaacaacccagtcagggatcgggaatgtcagaccgaaccgcctccgagaacgaacttctcggcgaccgcgaaccaatgggagatctacgacgcctacgtggaagaactggaaaagcaggaaaagactaaggaaaaggaaaaggccaagactcccgtcgccaagaagtcgggcaaaatggccatgcggaagctcacctccatggaatcacagactgacgacttgatcaagttgagccaggccgcaaagatcatggagcgcatggtcaaccaaaatacttacgacgatatcgcccaagacttcaagtactacgacgacgctgccgatgaataccgagatcaagtcggcaccctactgccgctttggaagttccagaatgacaaggccaagaggctgagcgtgaccgcgctgtgctggaaccccaaataccgcgacctcttcgccgtgggatacggctcctacgatttcatgaagcagagccggggaatgttgctcctttactccctgaagaacccctccttccctgagtacatgttcagctcaaacagcggcgtgatgtgcctcgacattcacgtggaccacccttacctcgtggccgtgggtcactacgacggcaacgtcgcgatctacaacttgaagaagccgcattcacagccctcgttttgctcctcggccaagtccggcaaacattcggacccagtgtggcaagtcaagtggcagaaagatgacatggaccaaaacttgaacttcttcagcgtgtcctccgacggacggatcgtgtcctggaccctcgtgaagcggaagttggtgcatatcgacgtgatcaaattgaaggtcgagggttcgaccaccgaagtgcctgaaggcctgcagcttcaccccgtgggatgcggcactgccttcgacttccacaaggagatcgactacatgttcctcgtgggaaccgaggaagggaagatctacaaatgcagcaagtcctactcatcacaattcctggatacctacgatgcccacaacatgagcgtggataccgtgtcgtggaacccctatcacaccaaggtattcatgtcctgctcctccgactggaccgtcaagatttgggaccacaccatcaagacccccatgttcatctacgacctgaactccgccgtgggggatgtggcctgggccccctactcgtcgaccgtgtttgccgcggtcaccacggacggaaaggcacacattttcgaccttgcgattaacaaatacgaggcgatttgcaaccagcccgtggccgccaaaaagaaccgcctgacccacgttcaattcaacttaatccacccaatcatcatcgtcggcgatgacagaggacacattattagcctgaaacttagccccaacctccgcaagatgcccaaggagaagaagggacaggaagtccagaagggccctgccgtggagattgcaaagctcgataagctcctgaacttagtccgggaagtgaagatcaagactgggggcggatcctacccctacgacgtgccagactacgcataa

SEQ ID NO: 14 - DNA, 2100 bp, Artificial Sequence
  Synthetic Polynucleotide
atgatccccgcctaagccaaagcccctcataagcaaccgcacaagcaaagcataagctgagggcggggtactcggaagcgtgacgaggactcgggaactgaagtaggagaggggaccgacgaatgggcgcagtcaaaggccaccgtgcgcccaccggaccagctcgagctgaccgatgctgagctgaaggaggagtttacccggatcctgacagccaacaacccacatgcaccgcagaacatcgtgcggtacagcttcaaagagggaacttataagcccattggcttcgtgaaccaactcgcggtgcattacacccaagtcggaaaccttattccgaaggactcggacgaaggcagacgccagcactaccgggacgagctcgtggcaggatcccaggaaagcgtcaaggtcatttccgagactggcaacctcgaggaggacgaagaacctaaggagctggaaaccgaacccggatcccagaccgacgtgccggccgctggggctgccgagaaagtcactgaagaggaactcatgaccccgaagcagccgaaagagagaaagctcaccaaccaattcaacttcagcgagcgcgccagccaaacctacaacaacccagtcagggatcgggaatgtcagaccgaaccgcctccgagaacgaacttctcggcgaccgcgaaccaatgggagatctacgacgcctacgtggaagaactggaaaagcaggaaaagactaaggaaaaggaaaaggccaagactcccgtcgccaagaagtcgggcaaaatggccatgcggaagctcacctccatggaatcacagactgacgacttgatcaagttgagccaggccgcaaagatcatggagcgcatggtcaaccaaaatacttacgacgatatcgcccaagacttcaagtactacgacgacgctgccgatgaataccgagatcaagtcggcaccctactgccgctttggaagttccagaatgacaaggccaagaggctgagcgtgaccgcgctgtgctggaaccccaaataccgcgacctcttcgccgtgggatacggctcctacgatttcatgaagcagagccggggaatgttgctcctttactccctgaagaacccctccttccctgagtacatgttcagctcaaacagcggcgtgatgtgcctcgacattcacgtggaccacccttacctcgtggccgtgggtcactacgacggcaacgtcgcgatctacaacttgaagaagccgcattcacagccctcgttttgctcctcggccaagtccggcaaacattcggacccagtgtggcaagtcaagtggcagaaagatgacatggaccaaaacttgaacttcttcagcgtgtcctccgacggacggatcgtgtcctggaccctcgtgaagcggaagttggtgcatatcgacgtgatcaaattgaaggtcgagggttcgaccaccgaagtgcctgaaggcctgcagcttcaccccgtgggatgcggcactgccttcgacttccacaaggagatcgactacatgttcctcgtgggaaccgaggaagggaagatctacaaatgcagcaagtcctactcatcacaattcctggatacctacgatgcccacaacatgagcgtggataccgtgtcgtggaacccctatcacaccaaggtattcatgtcctgctcctccgactggaccgtcaagatttgggaccacaccatcaagacccccatgttcatctacgacctgaactccgccgtgggggatgtggcctgggccccctactcgtcgaccgtgtttgccgcggtcaccacggacggaaaggcacacattttcgaccttgcgattaacaaatacgaggcgatttgcaaccagcccgtggccgccaaaaagaaccgcctgacccacgttcaattcaacttaatccacccaatcatcatcgtcggcgatgacagaggacacattattagcctgaaacttagccccaacctccgcaagatgcccaaggagaagaagggacaggaagtccagaagggccctgccgtggagattgcaaagctcgataagctcctgaacttagtccgggaagtgaagatcaagacttaa

SEQ ID NO: 15 - DNA, 2138 bp, Artificial Sequence
  Synthetic Polynucleotide
atgccatccaaacaaattagaaagcagagcattagcgtgacccgcggagcccggagaagagatgaggactcgggcaccgatgtcggcgaaggaaccgacgagtggtcgcagtcaaaggctaccgtgcggccgcctgaccagcttgaacttaccgatgccgagctgaaggaagagttcactcgcatcctgaccgccaacaatccccacgcgccgcagaacatcgtgcgctactccttcaaagaaggaacgtacaagctcattggattcgtgaaccaaatggccgtgcacttttcccaagtcggaaacctcattccgaaggattccgacgagggccggcggcagcattaccgcgacgaaatggtggccggatcgcaagaaagcatcaaggtcgtgacttccgaggccgagaacctcgaggaagaagaagaaccgaaagagggcgaaggagaagccgaagccgaggctgaggccggaagccagaccgatatccccgctgccgcagaaactaccgaaaaggtcatcgaagaggagttgatggccccggtgcagcctaaggaacggaagttgactaaccaattcaacttcagcgagagagcctcacagaccttcaacaacccactgagagatagggaatgtcaaatggagcctccgccccggacgaacttctcggccactgcaaaccaatgggagatctacgacgcttacgtggatgaactggagaagcaggaaaagaccaaggaaaaggagaaagccaagaccccggtcgccaaaaagactgagaaaatggccatgcggaagctcacttccatggagagccagtccgacgatattactaaggttacccaggccgccaagatcgtggaaagaatggtgaaccagaatacctacgacgacgtggcacaggattttaagtactatgaggacaccgctgacgaataccgggatcaagaaggcaccctcctgcccttgtggaagttccagaatgacaaggccaagaggctggccgtgactgcgctgtgctggaacccgaagtataaggacctcttcgccgtgggtcatggctcatacgactttatgaagcaatcgcgcgggatgctcctgctctactcgatgaagaacccttccttccccgagtacatgttcagctccgagtcagggatcatgtgcctggatgtccatgtcgaccacccttacctcgtggtcgtgggatattacgacggcaacgtggcgatctacaaccttaagaaaccgcatagccagccctgcttccggagcacctcaaagagcggaaagcacaccgaccccgtgtggcaagtcaagtggcaaaaggacgacatggaccacaacttgaacttcttcagcgtgtcgtccgacggacggatcgtgtcctggaccctcgtcaagtccgagctcgtgcacattgacattatcaagctcaaaaccgagggttccaccactgaaatccccgagggcctgcagctccataccgtgggatgcggcaccgcgttcgacttccataaagagatcgactacatgttcctcgtggggactgaagaggggaagatctacaagtgcagcaagtcgtactcttcccaattcctggacacctacgacgctcataacatggcagtggacgccgtgctgtggaacccataccacactaaggtctttatgtcctgctcctcggactggactgtgaagatttgggaccacaccatcaaaaccccaatgttcatttacgacctgaactccgccgtgggggatgtggcctgggccccgtattcctccaccgtattcgccgccgtgaccaccgacggaaaggcacacgtctttgacctggccgtgaacaaatacgaggcgatttgcaaccagccagtggtggccaagaagaaaaacaagatcactcacgtgcaattcaaccccatccaccccattattatcgtcggcgacgataggggtcacattatatgcctgaagctcagcccgaaccttcgcaagatgcctaaggaaaagaagggccaggaggtgcagaagggtcctgccgtcgaaattgcgaagttggacaagctcctgaacctcgtgcgcgaggtcaagactaagaccgggggtggttccgactacaaggatgatgacgataa

SEQ ID NO: 16 - DNA, 2144 bp, Artificial Sequence
  Synthetic Polynucleotide
atgccatccaaacaaattagaaagcagagcattagcgtgacccgcggagcccggagaagagatgaggactcgggcaccgatgtcggcgaaggaaccgacgagtggtcgcagtcaaaggctaccgtgcggccgcctgaccagcttgaacttaccgatgccgagctgaaggaagagttcactcgcatcctgaccgccaacaatccccacgcgccgcagaacatcgtgcgctactccttcaaagaaggaacgtacaagctcattggattcgtgaaccaaatggccgtgcacttttcccaagtcggaaacctcattccgaaggattccgacgagggccggcggcagcattaccgcgacgaaatggtggccggatcgcaagaaagcatcaaggtcgtgacttccgaggccgagaacctcgaggaagaagaagaaccgaaagagggcgaaggagaagccgaagccgaggctgaggccggaagccagaccgatatccccgctgccgcagaaactaccgaaaaggtcatcgaagaggagttgatggccccggtgcagcctaaggaacggaagttgactaaccaattcaacttcagcgagagagcctcacagaccttcaacaacccactgagagatagggaatgtcaaatggagcctccgccccggacgaacttctcggccactgcaaaccaatgggagatctacgacgcttacgtggatgaactggagaagcaggaaaagaccaaggaaaaggagaaagccaagaccccggtcgccaaaaagactgagaaaatggccatgcggaagctcacttccatggagagccagtccgacgatattactaaggttacccaggccgccaagatcgtggaaagaatggtgaaccagaatacctacgacgacgtggcacaggattttaagtactatgaggacaccgctgacgaataccgggatcaagaaggcaccctcctgcccttgtggaagttccagaatgacaaggccaagaggctggccgtgactgcgctgtgctggaacccgaagtataaggacctcttcgccgtgggtcatggctcatacgactttatgaagcaatcgcgcgggatgctcctgctctactcgatgaagaacccttccttccccgagtacatgttcagctccgagtcagggatcatgtgcctggatgtccatgtcgaccacccttacctcgtggtcgtgggatattacgacggcaacgtggcgatctacaaccttaagaaaccgcatagccagccctgcttccggagcacctcaaagagcggaaagcacaccgaccccgtgtggcaagtcaagtggcaaaaggacgacatggaccacaacttgaacttcttcagcgtgtcgtccgacggacggatcgtgtcctggaccctcgtcaagtccgagctcgtgcacattgacattatcaagctcaaaaccgagggttccaccactgaaatccccgagggcctgcagctccataccgtgggatgcggcaccgcgttcgacttccataaagagatcgactacatgttcctcgtggggactgaagaggggaagatctacaagtgcagcaagtcgtactcttcccaattcctggacacctacgacgctcataacatggcagtggacgccgtgctgtggaacccataccacactaaggtctttatgtcctgctcctcggactggactgtgaagatttgggaccacaccatcaaaaccccaatgttcatttacgacctgaactccgccgtgggggatgtggcctgggccccgtattcctccaccgtattcgccgccgtgaccaccgacggaaaggcacacgtctttgacctggccgtgaacaaatacgaggcgatttgcaaccagccagtggtggccaagaagaaaaacaagatcactcacgtgcaattcaaccccatccaccccattattatcgtcggcgacgataggggtcacattatatgcctgaagctcagcccgaaccttcgcaagatgcctaaggaaaagaagggccaggaggtgcagaagggtcctgccgtcgaaattgcgaagttggacaagctcctgaacctcgtgcgcgaggtcaagactaagaccgggggtggttcctacccatacgatgtgcctgactacgcgta